(19) United States
(12) Patent Application Publication
(10) Pub. No.: US 2022/0001007 A1
(43) Pub. Date: Jan. 6, 2022

(54) COMPOSITIONS AND METHODS

(71) Applicant: Oxford University Innovation Limited, Oxford (GB)

(72) Inventors: Sarah C. GILBERT, Oxford (GB); Sarah SEBASTIAN, Oxford (GB); Marta ULASZEWSKA, Oxford (GB); Adrian V.S. HILL, Oxford (GB); Teresa LAMBE, Oxford (GB)

(21) Appl. No.: 17/291,879

(22) PCT Filed: Nov. 5, 2019
(86) PCT No.: PCT/GB2019/053131

§ 371 Date: May 6, 2021

(30) Foreign Application Priority Data
Nov. 6, 2018 (GB) 1818084.4

(51) Int. Cl. A61K 39/25; A61K 35/761; A61P 31/22

(52) U.S. Cl. CPC A61K 39/25; A61P 31/22; A61K 35/761

(57) ABSTRACT

The invention relates to a viral vector comprising nucleic acid having a polynucleotide sequence encoding at least one epitope of the varicella-zoster virus (VZV) Gly E antigen, wherein said viral vector is an adenoviral vector. The invention also relates to uses, compositions for use in medical treatments, and methods of medical treatment.

### FIELD OF THE INVENTION

The invention relates to compositions useful in inducing immune responses against Varicella-Zoster Virus (VZV). In particular the invention relates to viral vectors comprising epitope(s) from VZV Gly E protein, such as adenoviral vectors comprising same.

### BACKGROUND TO THE INVENTION

Varicella-zoster virus (VZV or “Zoster Virus”) causes chicken pox, mainly in children. However, more importantly, the same virus can re-emerge in adults, usually decades after the primary chickenpox infection, causing the serious disease shingles.

Shingles, also known as herpes zoster, is an infection of a nerve and the skin around it. According to the NHS (the U.K.'s National Health Service), it is estimated that approximately one in every four people will have at least one episode of shingles during their life.

The main symptom of shingles is pain, followed by a rash that develops into itchy blisters, similar in appearance to chickenpox. New blisters may appear for up to a week, but a few days after appearing they become yellowish in colour, flatten and dry out. Scabs then form where the blisters were, which may leave some slight scarring and loss of skin pigment. The pain may be a constant, dull or burning sensation, and its intensity can vary from mild to severe. Patients may have sharp stabbing pains from time to time, and the affected area of skin will usually be tender. In some cases shingles may cause some early symptoms that develop a few days before the painful rash first appears, such as a headache, burning, tingling, numbness or itchiness of the skin in the affected area, a feeling of being generally unwell, and/or a high temperature (fever).

An episode of shingles typically lasts around two to four weeks. It usually affects a specific area on just one side of the body. It doesn't usually cross over the midline of the body. Any part of your body can be affected, including the face and eyes, but the chest and abdomen are the most common areas.

With life expectancy in the UK now above eighty years of age, long term health maintenance is a key aim of modern healthcare. Medical developments which specifically target impactful illnesses occurring more frequently in the older adult, such as shingles, will play a critical role in lowering the burden of disease and associated healthcare demands.

More than 90% of adults have been infected with varicella-zoster virus (VZV) and therefore are at risk of developing shingles (herpes zoster). Although shingles is most frequent in older adults (>50 years of age), it can occur at any age and especially in people who are immune-compromised (have a weakened immune system). Normally, the immune system can control the virus, but later in life VZV can be reactivated and result in shingles. It is not clear why this may happen but it could be linked to immunosenescence (a gradual deterioration of the immune system brought on by aging). The development of vaccines that specifically protect against illnesses that target the older adult is a key healthcare initiative.

It is desirable to reduce the severity of symptoms and/or the risk of developing complications. Complications of shingles can include meningitis or encephalitis; if shingles affects the eye(s) there is a risk of developing permanent vision problems if the condition isn't treated quickly.

EP3210631 discloses a DNA vaccine composition for preventing and treating herpes zoster, and method for activating T cells for VZV antigen by using same. This document describes a DNA vaccine composition for preventing and treating herpes zoster, containing: at least one type of plasmid containing the insertion site of a varicella-zoster virus (VZV)-derived gene encoding a VZV protein; and other pharmaceutically acceptable ingredients. There is no mention of viral vectors in this document.

WO2014/043189 discloses conditionally replication deficient herpes viruses and use thereof in vaccines. Creation of variant or mutagenised herpes viruses and host cells containing rendered conditionally replication defective by the incorporation or fusion of one or more destabilization domains onto one or more genes which are essential for viral replication are described. There is no mention of viral vectors in this document.

EP1721981 discloses recombinant varicella-zoster virus prepared using BAC (E. coli artificial chromosome), and a pharmaceutical composition comprising such a virus. The focus of this document is on identification of non-essential regions in the VZV, in particular wherein the non-essential region is the region flanking the ORF of gene 11, or the region flanking the ORF of gene 12. There is no mention of viral vectors carrying VZV antigen(s) in this document.

WO2009/012486 discloses varicella zoster virus-virus like particles (VLPS) and antigens. In particular this document describes a purified virus like particle (VLP) from Varicella Zoster Virus (VZV) comprising VZV gE protein, but does not include VZV nucleic acid or a yeast Ty protein. The focus of this document is on VLPs further comprising at least one additional protein from an infectious agent. The only mention of viral vectors in this document is in paragraphs 0048-0049 as general expression vectors.

Prior art vaccines against this virus include Zostavax™ (made by Merck). The protection from Zostavax™ is mainly via the antibody response. The European Medicines Agency (EMA) document WC500053460 discusses Zostavax, and it is asserted in the art that the correlation between immune responses and protection against Herpes Zoster (HZ) were observed with gpELISA measurements, while, the results of VZV IFN-γ ELISPOT test had a less clear correlation to the protection. Zostavax™ is typically given in the UK to all adults at age 70. However, the vaccine is not fully effective, and its usefulness against shingles decreases with age (from 69.8% in adults between the ages of 50-59 years, to 37.6% in those ≥70 years of age). Thus the efficacy is 30-40% which is very poor. This is a problem in the art. In addition, the protection given by Zostavax™ is typically 5 years or less, which is problematically short.

Furthermore, the vaccine is not recommended for people with weakened immune systems who are at an increased risk of developing shingles (e.g. patients with HIV). There is, therefore, an unmet need for a vaccine that gives improved protection across all ages, but especially in elderly and immunocompromised populations.

Zostavax™ is a live attenuated virus. Therefore, when given to humans, it causes a limited infection which boosts the immune response in humans without causing the shingles disease. It should be noted that this preparation does not replicate in mice, so when given to mice it is more similar to giving a replication defective virus.

SHINGRIX™ is a vaccine indicated for prevention of herpes zoster (shingles) in adults aged 50 years and older. SHINGRIX™ is manufactured by GlaxoSmithKline Biologicals, Rixensart, Belgium. SHINGRIX™ is prepared by reconstituting a lyophilized varicella zoster virus glycoprotein E (gE) antigen component with an accompanying AS01B adjuvant suspension component. Thus, SHINGRIX™ is a protein vaccine based on the Gly E antigen. This has to be given as 2 administrations in order to be effective. Each administration has to be given with an adjuvant such as the AS01 adjuvant. This adjuvant is a reactogenic, which can be uncomfortable for patients—85% of recipients report pain on injection. Side effects of SHINGRIX™ which may occur include redness, itching, swelling, warmth, bruising, or pain at the injection site. Headache, muscle pain, tiredness, or fever may also occur. Moreover, the reactogenicity can cause a secondary problem of compliance. This is because the reaction experienced after administering the vaccine tends to put patients off from returning for their second dose, yet the second dose is needed in order to complete the recommended regimen and associated level of protection. This is a problem in the art.

A further drawback with prior art approaches such as the SHINGRIX™ vaccine is that it requires two vials of material to be stored and mixed at the point of administration—in the case of SHINGRIX™ this is a vial of adjuvant and a vial of antigen which are formulated into a single mixture at the point of administration.

In the US, the list price for Zostavax is $196.91 per dose in the private sector and $117.12 per dose for CDC vaccine contracts. In terms of reimbursement, Zostavax is covered by most private health insurers in the US (98%) for adults age ≥60 years (although some plans may require patient co-pay). The vaccine is also covered by Medicare for adults aged ≥65 years, although it would be under Part D (which often has co-pay requirements and out-of-pocket expenses). Moreover, commentators identify Shingrix's 2-dose administration and its tolerability profile to be potential weaknesses. Thus there are persisting problems with prior art approaches.

The invention seeks to overcome problem(s) associated with the prior art.

### SUMMARY OF THE INVENTION

Lack of adequate cell-mediated immunity (CMI) to varicella-zoster virus (VZV) has been associated with higher risks of developing shingles and associated pain.

The invention describes an adenovirus—Gly E zoster virus vaccine. It is shown to induce a T-cell response. These vectors can be used in prime boost vaccination regimes. A strong T-cell response is demonstrated by data provided in this application. Thus, the invention provides an advantageous, strong and maintained T-cell response.

Varicella zoster virus (VZV) causes chickenpox and Zoster (shingles). Chickenpox is a highly contagious disease caused by the initial infection with varicella zoster virus (VZV). Chickenpox is one of the most common childhood diseases and is characterised by a blister-like rash and fever, with more than 90% of the population being exposed during the first two decades of life. Although chickenpox is generally a mild self-limiting illness, in immunocompromised subjects and adults it can be more serious. Zoster or shingles is caused by the reactivation of VZV persisting in a latent form in the dorsal sensory ganglia. Prevention of chickenpox through vaccination is a desirable medicinal intervention.

It should be noted that the compositions and/or vaccines described herein are not therapeutic i.e. they are not taught as eliminating/eradicating virus. They are taught as vaccine compositions for use in maintaining control of VZV infection(s) and/or preventing resurgence of replicative VZV infection causing shingles. In other words, the compositions are taught as vaccine compositions for use in induction of immune responses from the host organism, not as agents directly acting on the virus itself. The compositions as useful to induce protection against an initial infection as in chickenpox, and/or to induce protection against reactivation of a latent virus (sometimes called ‘dormant virus’) as in shingles.

Suitably references to ‘existing infection’ mean ‘latent infection’ or ‘static infection’ i.e. virus in the lysogenic phase of the lifecycle i.e. a dormant VZV infection (defined as one that is no longer causing an active infection).

Suitably references to ‘infection’ have their normal meaning in the art, i.e. active infection or productive virus infection causing disease such as chickenpox or shingles, most suitably shingles. ‘Infection’ would normally have associated viraemia i.e. active infection (rather than latent infection as discussed above).

Suitably the compositions described herein are for use in prevention of resurgent VZV infection, suitably for use in prevention of replicative VZV infection, suitably for use in prevention of disease(s) caused by reactivation of latent VZV infection.

Without wishing to be bound by theory, the inventors believe that the resurgence of the virus in adults causing shingles can be because of waning T-cell responses/waning number of T-cells against the virus in circulation. For this reason, the inventors teach for the first time that existing approaches (which are based mainly on the antibody response) may not be fit for purpose. For these reasons, the inventors teach the viral vectored constructs as set out in the claims which have the advantage of inducing strong cellular immune responses, for example T-cell responses, and thereby protecting the recipients.

In a broad aspect, the invention relates to a viral vector comprising nucleic acid having a polynucleotide sequence encoding at least one epitope of the varicella-zoster virus (VZV) Gly E antigen. Suitably the viral vector and the varicella-zoster virus (VZV) Gly E antigen are heterologous i.e. suitably the viral vector is not, or is not derived from, VZV.

Suitably the invention relates to a viral vector comprising nucleic acid having a polynucleotide sequence encoding at least one epitope of the varicella-zoster virus (VZV) Gly E antigen, wherein said viral vector is an adenoviral vector.

In one aspect, the invention relates to a composition comprising a viral vector comprising nucleic acid having a polynucleotide sequence encoding at least one epitope of the varicella-zoster virus (VZV) Gly E antigen, wherein said viral vector is an adenoviral vector.

Suitably said at least one epitope comprises at least one CD4 T cell epitope and at least one CD8 T cell epitope.

Suitably said adenoviral vector is of human or simian origin.

Suitably said adenoviral vector is ChAdOx 1 or ChAdOx 2.

Suitably said composition is adjuvant-free.

Suitably said Gly E antigen has the amino acid sequence of SEQ ID NO: 1 or SEQ ID NO: 2, suitably SEQ ID NO: 2.

Suitably said polynucleotide sequence comprises the sequence of SEQ ID NO: 3 or SEQ ID NO: 4, suitably SEQ ID NO: 4.

Suitably said polynucleotide sequence further comprises the sequence of the bgh polyadenylation signal SEQ ID NO: 6.

Suitably said polynucleotide sequence encoding at least one epitope of the varicella-zoster virus (VZV) Gly E antigen is operably connected to the long CMV promoter, suitably the long CMV promoter has the nucleotide sequence of SEQ ID NO: 7.

Suitably said viral vector sequence is as in ECACC accession number 12052403 (ChAdOx1).

Suitably said viral vector sequence is as in SEQ ID NO: 5 (ChAdOx2).

Suitably administration of a single dose of said composition to a mammalian subject induces protective immunity in said subject.

Suitably the composition as described above is formulated such that administration of a single dose of said composition to a mammalian subject induces protective immunity in said subject.

Suitably the composition as described above is for induction of an immune response against VZV.

Suitably said immune response is a cellular immune response. Suitably said cellular immune response comprises a NK cell response and/or a T cell response. Suitably said cellular immune response comprises a T cell response. Suitably said T cell response comprises a CD8+ T cell response. Suitably said T cell response comprises a CD4+ T cell response. More suitably said T cell response comprises a CD8+ and a CD4+ T cell response. Most suitably said T cell response comprises a CD4+ T cell response. Suitably said T cell response comprises a triple secreting CD4+ T cell response.

Suitably the composition as described above is for induction of an immune response against VZV, wherein a single dose of said composition is administered.

Suitably the composition as described above is for induction of an immune response against VZV, wherein said composition is administered once.

The invention advantageously provides a composition which has the advantage of being effective when administered only once. However, in one embodiment, if the immune response in a subject wanes over time then suitably the composition may be administered (readministered) to said subject. For example, said composition may be administered every 5 years, more suitably once every year.

Suitably the composition as described above is for induction of an immune response against VZV, wherein said composition is administered once per 5 years, more suitably once per year.

Suitably the composition as described above is for preventing VZV infection.

Suitably the composition as described above is for prevention of shingles.

Suitably the composition as described above is for preventing VZV infection, or for prevention of shingles, wherein a single dose of said composition is administered.

Suitably the composition as described above is for preventing VZV infection, or for prevention of shingles, wherein said composition is administered once.

Suitably the composition as described above is for use in preventing VZV infection.

Suitably the composition as described above is for use in prevention of shingles.

Suitably the composition as described above is for use in preventing VZV infection, or for use in prevention of shingles, wherein a single dose of said composition is administered.

Suitably the composition as described above is for use in preventing VZV infection, or for use in prevention of shingles, wherein said composition is administered once.

In one aspect, the invention relates to use of a composition as described above in medicine.

In one aspect, the invention relates to use of a composition as described above in the preparation of a medicament for prevention of VZV infection.

In one aspect, the invention relates to use of a composition as described above in the preparation of a medicament for prevention of shingles.

In one aspect, the invention relates to use of a composition as described above in the preparation of a medicament for induction of, or that induces, both CD4+ and CD8+ T cell responses to Gly E antigen in a subject. Suitably said medicament further induces antibodies to Gly E antigen in said subject.

In one aspect, the invention relates to a method of inducing an immune response against varicella-zoster virus (VZV) in a mammalian subject, the method comprising administering a composition as described above to said subject.

In one aspect, the invention relates to a method of preventing shingles in a mammalian subject, the method comprising administering a composition as described above to said subject.

Suitably a single dose of said composition is administered to said subject.

Suitably said composition is administered once.

Suitably said composition is administered once per 5 years, more suitably once per year.

Suitably said composition is administered by a route of administration selected from a group consisting of subcutaneous, intradermal and intramuscular. Suitably said administration is intramuscular.

In one embodiment suitably the composition as described above is for treatment or prevention of chickenpox.

### DETAILED DESCRIPTION OF THE INVENTION

The inventors have used an innovative technology, viral vectored vaccines to protect against infectious disease. The inventors use their approach to ‘re-purpose’ a virus by inserting a small part of a different virus (the one that causes the target disease) into a virus vectored backbone. These ‘recombined’ viral vaccines can't replicate and will not cause disease—but can induce a strong immune response toward the inserted or foreign virus segment. The inventors have demonstrated that viral vectored vaccines are safe and can effectively induce an immune response in the older adult and in immune-compromised individuals (HIV infected)—both key populations at risk of developing shingles.

Here we teach viral vectored vaccines toward VZV (the underlying causative agent in shingles) and we test these vaccines and show that they can induce an immune response in appropriate models.

In the art, recent data suggest that a newly developed vaccine (Shingrix™—adjuvanted protein) toward shingles can induce protective efficacy toward shingles, if given as repeated immunizations. Unfortunately, the main use of adjuvants (i.e. to induce strong immune responses) can as a corollary induce adverse reactions, which is a problem in the art. Advantageously, whilst viral vectored vaccines of the invention can induce similarly strong immune responses, limited or minimal adverse reactions are seen post-vaccination.

We have generated viral vectored vaccines toward VZV tested that these vaccines can induce an immune response in appropriate models.

We teach various vaccination regimens to induce strong and long-lived immunity toward VZV.

We have generated VZV-vaccines and vaccination studies have demonstrated stronger cell-mediated immunity (CMI) than that achieved with the current licensed vaccine known in the art.

As used herein, the term ‘about’ means +/−1% of the value given.

The invention provides vectors (suitably viral vectors, most suitably adenoviral vectors), compositions and formulations (such as pharmaceutical compositions, such as medicaments, such as vaccines) suitable for inducing an immune response, suitably a T cell mediated immune response, against a varicella zoster virus (VZV) in a vertebrate subject (suitably a mammal, more suitably a primate, most suitably a human).

Suitably the immune response comprises a cell mediated response.

Suitably the immune response comprises cell mediated immunity (CMI).

Suitably the immune response comprises induction of CD4+ T cells.

Suitably the immune response comprises induction of a CD4+ cytotoxic T cell (CTL) response.

In one embodiment the immune response comprises both a humoral response and a cell mediated response.

Suitably the immune response comprises protective immunity.

Suitably vector(s) of the invention comprise nucleic acid having polynucleotide sequence encoding one or more epitopes of the antigen of interest. In one embodiment suitably vector(s) of the invention comprise nucleic acid having polynucleotide sequence which is the complement of nucleotide sequence encoding one or more epitopes of the antigen of interest.

Suitably the one or more epitope(s) is/are T cell epitope(s). Suitably the one or more epitope(s) is/are CD4+ T cell epitope(s). Suitably the one or more epitope(s) is/are CD8+ T cell epitope(s). Suitably the one or more epitope(s) comprise at least one CD4+ T cell epitope and at least one CD8+ T cell epitope.

Suitably vector(s) of the invention comprise nucleic acid having polynucleotide sequence encoding CD4 T cell epitopes of GlyE.

Suitably vector(s) of the invention comprise nucleic acid having polynucleotide sequence encoding CD8 T cell epitopes of GlyE.

Suitably vector(s) of the invention comprise nucleic acid having polynucleotide sequence encoding both CD4 and CD8 T cell epitopes of GlyE.

Suitably the vector is used to induce both CD4 and CD8 T cell responses to GlyE (especially in humans); most suitably to induce both CD4 and CD8 T cell responses to GlyE (especially in humans) in addition to antibodies.

This further distinguishes the ChAd vaccines of the invention from prior art such as Shingrix. Moreover this shows another property of the invention that is distinctive and improved over the prior art.

Adenoviral vectors have DNA genomes. Thus the nucleic acid is suitably DNA, most suitably dsDNA.

Suitably the adenoviral vector is of simian or human origin; suitably the adenoviral vector is of chimpanzee or human origin; suitably the adenoviral vector is of chimpanzee origin.

Suitably the nucleotide sequence is DNA sequence.

In one embodiment we provide one of the main VZV surface antigens, glycoprotein E, cloned into a ChAdOx1 viral vector backbone. Expression is driven by the long CMV promoter. The use of chimp-derived viral vectored vaccines to augment immune responses toward VZV gpE has not been done before, to the best of the inventors' knowledge. Critically, we have also demonstrated that viral vectored vaccines are safe and can effectively induce an immune response in the older adult and in immune-compromised individuals (HIV infected)—both key populations at risk of developing shingles.

Shingles (Herpes Zoster) and Chickenpox (Varicella)

As noted above, shingles (sometimes referred to as ‘Herpes Zoster’) is caused by varicella zoster virus (VZV), the same virus that causes chickenpox. Most people have chickenpox in childhood, but after the illness has resolved the varicella-zoster virus remains inactive (dormant) in the nervous system. The immune system keeps the virus in check, but the VZV can be reactivated later in life and cause shingles.

Without wishing to be bound by theory, it is not well understood why the shingles virus is reactivated at a later stage in life, but most cases are thought to be caused by having lowered immunity.

In more detail, infection with varicella-zoster virus (VZV), an alpha herpesvirus, is associated with two distinct diseases; varicella and herpes zoster. Primary infection results in chickenpox (varicella) a generally mild, self-limiting illness usually acquired in childhood or adolescence and affecting almost all individuals. Following initial primary infection with VZV, the virus remains latent in the dorsal root ganglia. It is assumed that latent virus may frequently reactivate and replicate subclinically. These episodes of transient subclinical viremia lead to repeated antigenic stimulation of immunity without clinical manifestations of disease. In some individuals, however, reactivation and replication of the latent virus result in the clinical manifestation of herpes zoster (HZ), which is often referred to as shingles. HZ is characterized by a unilateral, vesicular rash with a dermatomal distribution that generally corresponds to the area of skin innervated by a single spinal or cranial sensory ganglion. Typically, the vesicles crust over in 7 to 10 days, but may take up to a month to heal. One of the most significant clinical manifestations of HZ is pain, which is considered to be due to VZV induced neuronal destruction and inflammation. HZ-related pain may occur during 3 time periods: —prior to onset of the cutaneous eruption (prodromal pain, typically beginning 3 to 5 days prior to the appearance of skin lesions): —during the period of the acute rash (acute neuritis), and following healing of the acute skin lesions; —beyond cutaneous healing for a prolonged period of time (postherpetic neuralgia, PHN). PHN, the most severe sequelae of HZ, occurs in 10-20% of HZ patients and is described by characteristic patterns of pain with the majority of patients experiencing the following patterns—constant pain described as burning, throbbing or aching pain; —intermittent sharp, stabbing, shooting, lancinating pain; —stimulus-evoked pain as allodynia that usually lasts well beyond the duration of the stimulus. Allodynia, which is present in at least 90% of PHN patients, is typically described as the most distressing and debilitating component of HZ.

The mechanisms leading to HZ are not well understood, however, one predisposing factor in developing HZ in immunocompetent persons is advancing age. The incidence and severity of HZ increase from 2.5 per 1000 person-years in adults aged 20-50 years to 7.8 per 1000 person-years in those aged >60 years. Furthermore, complications such as PHN, which are relatively infrequent in otherwise healthy children and younger adults, occur in almost one-half of older individuals. It is postulated that the age-related increase in the risk of HZ among otherwise healthy elderly subjects is attributed to immunosenescence and has been correlated with a diminished cell-mediated immunity (CMI), but not with the level of circulating VZV specific serum antibodies. However, studies conducted in immunocompromised patients indicate, that low or absent CMI represents a necessary, but not a sufficient condition for the occurrence of HZ.

Viral Vectors

Suitably the viral vector (sometimes referred to as ‘vector’) is an adenoviral vector.

Suitably said coding sequence is present in an adenovirus based vector. In other words, suitably said coding sequence is present in an adenoviral vector.

Any suitable adeno-based viral vector may be used.

The adenoviral vector of the invention may be any adenoviral vector suitable for use in humans.

In more detail, any replication-deficient viral vector, for human use preferably derived from a non-human adenovirus may be used. For veterinary use Ad5 may be used.

Suitably the vector may be ChAdOx1.

Suitably the vector may be ChAdOx2.

ChAdOx1

ChAdOx1 is described in patent application number WO2012/172277. In brief ChAdOx1 is derived from the “Y25” chimpanzee adenovirus isolate. A replication deficient vector derived from Y25 was taken and the E1 and E3 genes were deleted. In order to improve yields, some ORFs in E4 were replaced with the corresponding ORFs from human adenovirus 5 (three such ORFs were replaced) which lead to better yields. E4 is involved with viral replication and is not believed to affect immunogenicity/safety.

In more detail, ChAdOx1 is described in Dicks M D J, Spencer A J, Edwards N J, Wadell G, Bojang K, et al. (2012) A Novel Chimpanzee Adenovirus Vector with Low Human Seroprevalence: Improved Systems for Vector Derivation and Comparative Immunogenicity. PLoS ONE 7(7): e40385, and in WO2012/172277. Both these documents are hereby incorporated herein by reference, in particular for the specific teachings of the ChAdOx1 vector, including its construction and manufacture.

In addition, a clone of ChAdOx1 containing GFP is deposited with the ECACC: a sample of E. coli strain SW1029 (a derivative of DH10B) containing bacterial artificial chromosomes (BACs) containing the cloned genome of AdChOX1 (pBACe3.6 AdChOx1 (E4 modified) TIPeGFP, cell line name “AdChOx1 (E4 modified) TIPeGFP”) was deposited by Isis Innovation Limited on 24 May 2012 with the European Collection of Cell Cultures (ECACC) at the Health Protection Agency Culture Collections, Health Protection Agency, Porton Down, Salisbury SP4 oJG, United Kingdom under the Budapest Treaty and designated by provisional accession no. 12052403. Isis Innovation Limited is the former name of the proprietor/applicant of this patent/application.

ChAdOx2

ChAdOx2 is described in patent application WO2017/221031. Similar to ChAdOx1, ChAdOx2 is derived from a C68 isolate of chimpanzee adenovirus. Again a replication defective virus was obtained and the E1 and E3 genes were deleted. The replacement of three E4 ORFs as conducted on ChAdOx1 presented challenges when implemented on ChAdOx2. Therefore, the whole E4 region of ChAdOx2 was replaced with the engineered E4 region of ChAdOx1 (as described above).

In more detail, The nucleotide sequence of the ChAdOx2 vector (with a Gateway™ cassette in the E1 locus) is shown in SEQ ID NO. 5 This is a viral vector based on Chimpanzee adenovirus C68. (This is the sequence of SEQ ID NO: 10 in GB patent application number 1610967.0—the priority application for WO2017/221031).

In addition, a clone of ChAdOx2 containing GFP is deposited with the ECACC: deposit accession number 16061301 was deposited by Isis Innovation Limited on 13 Jun. 2016 with the European Collection of Cell Cultures (ECACC) at the Health Protection Agency Culture Collections, Health Protection Agency, Porton Down, Salisbury SP4 oJG, United Kingdom under the Budapest Treaty. Isis Innovation Limited is the former name of the proprietor/applicant of this patent/application.

Therefore, because ChAdOx1 and ChAdOx2 are different to some degree, they can be used together in heterologous prime boost regimes (e.g. a ChAdOx1 prime followed by a ChAdOx2 boost, or a ChAdOx2 prime followed by a ChAdOx1 boost). Of course, either of these vectors may be used in conventional heterologous prime boost regimes for example adenovirus prime followed by pox virus boost, or pox virus prime followed by adenovirus boost.

Manufacture of vaccine doses from ChAdOx1/ChAdOx2 is identical. Therefore, techniques described herein for manipulation/amplification/preparation of ChAdOx vectors may apply equally to ChAdOx1 and/or ChAdOx2.

Whilst the general principles of manufacture are identical, there will be some minor differences in the exact conditions for downstream processing due to slightly different charge on the virions. This is well within routine variations for a person skilled in the art of virus production.

In brief, manufacture/harvest/purification of viral vectors for compositions of the invention is suitably carried out under GMP (Good Manufacturing Practice) conditions. The viral vectors of the present invention may be produced in engineered cell lines containing a complement of any deleted genes required for viral replication. The adenoviral vectors according to the present invention suitably further comprise one or more modifications designed to optimise vector growth and yield in transformed cell lines, such as HEK293, expressing the genes functionally deleted in the adenoviral vector according to the present invention. Manufacture of adenoviral vectors is well known in the art. In particular, precise conditions for production of adenoviral vectors such as the ChAdOx1 and ChAdOx2 vectors, are described in prior art such as WO2012/172277 or WO2017/221031.

### FORMULATION

The formulation buffer, as used for the clinical product is:

Formulation Buffer Components
1. 10 mM Histidine
2. 7-5% Sucrose
3. 35 mM Sodium chloride
4. 1 mM Magnesium chloride
5. 0.1% Polysorbate 80
6. 0.1 mM EDTA
7. 0.5% Ethanol
8. Hydrochloric acid (for pH adjustment to ˜pH 6.6)

Formulated in water for injection Ph Eur.

Other formulations may be used, for example Alternative buffer e.g. Merck Formulation Buffer A195 (10 mM Tris, 10 mM Histidine, 5% sucrose, 75 mM NaCl, 1 mM MgCl₂, 0.02% PS-80, 0.1 mM EDTA, 0.5% EtOH, pH 7.4).

Formulations for other administration routes such as aerosol will be adjusted accordingly by the skilled operator.

Suitably the composition and/or formulation does not comprise adjuvant. Suitably adjuvant is omitted from the composition and/or formulation of the invention.

Antigen Insertion

For insertion of the nucleotide sequence encoding Gly E antigen, suitably the E1 site may be used, suitably with the hCMV IE promoter. Insertion into the E1 site is well within the ambit of the skilled reader; in the event that any guidance was needed reference is made to the description of the ChAdOx1 and ChAdOx2 vectors (see above), and/or to WO2012/172277 or WO2017/221031. Suitably the short or the long version of the hCMV IE promoter may be used; most suitably the long version as described in WO2008/122811, which is specifically incorporated herein by reference for the teaching of the promoters, particularly the long promoter.

It is also possible to insert antigens at the E3 site, or close to the inverted terminal repeat sequences, if desired.

Antigen Expression

Antigen may be constitutively expressed from viral vectors. Indeed, the inventors have shown that viral vectors described herein constitutively expressing the antigen are stable through numerous passages. This is an advantage of the invention. However, if desired, the expression of the antigen may be repressed during manufacture which may lead to better yields and/or may avoid problems with antigen toxicity. This is a matter for operator choice.

Data presented herein includes ELISPOT data showing T-cell responses. This is a departure from prior art approaches where zoster virus vaccines have been primarily focused on the antibody response.

Varicella-Zoster Virus (VZV)

In another aspect, the invention relates to a vector, composition or medicament as described herein for treatment of VZV infection. By ‘treatment’ is meant control or prevention of resurgence e.g. from dormant virus (sometimes referred to as ‘endogenous virus’ in mammals such as primates e.g. humans).

Suitably the vector, composition or medicament of the invention is for controlling reactivation of VZV.

Suitably the vector, composition or medicament is for preventing resurgence of VZV infection.

Suitably the vector, composition or medicament is for controlling shingles.

Suitably the vector, composition or medicament is for preventing shingles.

### Advantages

A drawback with prior art approaches such as the SHINGRIX™ vaccine is that it requires two vials of material to be stored and mixed at the point of administration—in the case of SHINGRIX™ this is a vial of adjuvant and a vial of antigen which are formulated into a single mixture at the point of administration. In contrast, the present invention advantageously requires only a single vial of material to be stored/transported/manipulated.

Furthermore, it is an advantage of the invention that superior immunogenicity is delivered compared to either Zostavax or SHINGRIX™ prior art vaccines.

It is an advantage of the invention that only a single dose is needed.

It is an advantage of the invention that only a single dose is needed to induce an immune response.

It is an advantage of the invention that the vectors are safe in immune compromised subjects.

It is an advantage of the invention that a better response is observed compared to prior art approaches.

It is an advantage of the invention that T-cell responses are generated, in particular CD4+ T-cell responses.

In some embodiments it is an advantage of the invention that CD8+ T-cell responses are also generated; in some embodiments it is an advantage of the invention that strong antibody responses are also generated; most importantly the invention provides the advantage of generating/enhancing CD4+ T-cell responses.

It is an advantage of the invention that a strong CD8+ T cell response is produced.

A ‘good’ single shot Ad vaccination will give a response in the 100's; a boost is generally required to get above 1,000 SFU. Thus ‘strong’ suitably means >800 SFU after a single 30 shot (single administration). Of course the skilled reader will appreciate that this can be dose dependent—these comments are in the context of the preferred dose given herein.

For example, it is an advantage of the invention that a strong T cell response is 35 measured even after two weeks following one shot of ChAdOx1-VZV GpE with a mean response of 1361 (s.e.m. 145 n=5) SFU per 10⁶ splenocytes produced (N.B. ELISpot will measure CD4+ and CD8+ T cell responses). Previous work with these viral vectors has demonstrated lower immune responses following one-shot immunisation against variant antigen inserts, for example one shot vaccination with monovalent EBOV in preclinical models induces only 200-500 SFU—showing that the invention produces a much stronger response than the prior art. Moreover, FIG. 1 and FIG. 2 from prior art Dicks et al 2015 (Vaccine 33 (2015) pages 1121-1128 “The relative magnitude of transgene-specific adaptive immune responses induced by human and chimpanzee adenovirus vectors differs between laboratory animals and a target species”) show much lower immunogenicity, again demonstrating the surprising strength of the responses according to the present invention.

It is an advantage of the invention that a sustained T cell response is produced. Unless otherwise apparent from the context, ‘sustained’ means at least 16 weeks.

It is an advantage of the invention that the same vectors can be used to re-vaccinate (i.e. to boost) patients. This may be obtained by priming with ChAdOx1 and boosting with ChAdOx2, or priming with ChAdOx2 and boosting with ChAdOx1. Moreover, the same vector may be used for a boost as used for a prime if the boost is carried out at an interval of at least 6 months from the prime. This may be referred to as “homologous prime-boost”.

It is an advantage of the invention that no adjuvant is required. This avoids the disadvantage of the pain/reactogenicity which is experienced when using adjuvants in administration to humans.

It is an advantage of the invention that the compositions are cheaper than adjuvanted vaccines. Adjuvants are complex preparations and can be expensive, such as 20 USD per administration. The compositions of the invention require only a single component (i.e. the viral vector containing the antigen as described) and are therefore simpler and cheaper, which is an advantage of the invention.

It is an advantage of the invention that live replication deficient viral vectors are used, so the invention is safer than prior art such as Zostavax™.

It is an advantage of the invention that only a single dose is required, so the invention is better than multi-dose prior art such as Shingrix™.

It is an advantage of the invention that only a single composition/single vial is needed, so the invention is better than adjuvanted prior art such as Shingrix™ which requires two vials, one of antigen and one of adjuvant, to be transported and stored, and then mixed immediately before injection.

It is an advantage of the invention that immunogenicity is superior to prior art such as Zostavax™ or Shingrix™.

Gly E Antigen

By “Gly E antigen” (sometimes referred to as “gE”) is meant the “standard” gE antigen sequence of VZV.

In more detail, the original VZV sequence (and strain) used to make the compositions of the invention is suitably the publically disclosed coding sequence as follows: DEFINITION Human herpesvirus 3 isolate 1140VZV glycoprotein E gene, complete cds

```
ACCESSION (GenBank) AY253715
VERSION AY253715.1:
      1 atggggacag ttaataaacc tgtggtgggg gtattgatgg
ggttcggaat tatcacggga
    61 acgttgcgta taacgaatcc ggtcagagca tccgtcttgc
gatacgatga ttttcacatc
  121 gatgaagaca aactggatac aaactccgta tatgagcctt
actaccattc agatcatgcg
  181 gagtcttcat gggtaaatcg gggagagtct tcgcgaaaag
cgtacgatca taactcacct
  241 tatatatggc cacgtaatga ttatgatgga tttttagaga
acgcacacga acaccatggg
  301 gtgtataatc agggccgtgg tatcgatagc ggggaacggt
taatgcaacc cacacaaatg
  361 tctgcacagg aggatcttgg ggacgatacg ggcatccacg
ttatccctac gttaaacggc
  421 gatgacagac ataaaattgt aaatgtggac caacgtcaat
acggtgacgt gtttaaagga
  481 gatcttaatc caaaacccca aggccaaaga ctcattgagg
tgtcagtgga agaaaatcac
  541 ccgtttactt tacgcgcacc gattcagcgg atttatggag
tccggtacac cgagacttgg
  601 agctttttgc cgtcattaac ctgtacggga gacgcagcgc
ccgccatcca gcatatatgt
  661 ttaaaacata caacatgctt tcaagacgtg gtggtggatg
tggattgcgc ggaaaatact
  721 aaagaggatc agttggccga aatcagttac cgttttcaag
gtaagaagga agcggaccaa
  781 ccgtggattg ttgtaaacac gagcacactg tttgatgaac
tcgaattaga cccccccgag
  841 attgaaccgg gtgtcttgaa agtacttcgg acagaaaaac
aatacttggg tgtgtacatt
  901 tggaacatgc gcggctccga tggtacgtct acctacgcca
cgtttttggt cacctggaaa
  961 ggggatgaaa aaacaagaaa ccctacgccc gcagtaactc
ctcaaccaag aggggctgag
1021 tttcatatgt ggaattacca ctcgcatgta ttttcagttg
gtgatacgtt tagcttggca
1081 atgcatcttc agtataagat acatgaagcg ccatttgatt
tgctgttaga gtggttgtat
1141 gtccccatcg atcctacatg tcaaccaatg cggttatatt
ctacgtgttt gtatcatccc
1201 aacgcacccc aatgcctctc tcatatgaat tccggttgta
catttacctc gccacattta
1261 gcccagcgtg ttgcaagcac agtgtatcaa aattgtgaac
atgcagataa ctacaccgca
1321 tattgtctgg gaatatctca tatggagcct agctttggtc
taatcttaca cgacgggggc
1381 accacgttaa agtttgtaga tacacccgag agtttgtcgg
gattatacgt ttttgtggtg
1441 tattttaacg ggcatgttga agccgtagca tacactgttg
tatccacagt agatcatttt
1501 gtaaacgcaa ttgaagagcg tggatttccg ccaacggccg
gtcagccacc ggcgactact
1561 aaacccaagg aaattacccc cgtaaacccc ggaacgtcac
cacttctacg atatgccgca
1621 tggaccggag ggcttgcagc agtagtactt ttatgtctcg
taatattttt aatctgtacg
1681 gctaaacgaa tgagggttaa agcctatagg gtagacaagt
ccccgtataa ccaaagcatg
1741 tattacgctg gccttccagt ggacgatttc gaggactcgg
aatctacgga tacggaagaa
1801 gagtttggta acgcgattgg agggagtcac gggggttcga
gttacacggt gtatatagat
1861 aagacccggt ga
```

It is the same coding sequence to the following most commonly known VZV strain: Human herpesvirus 3 strain Oka vaccine strain.

Suitably the GlyE has the amino acid sequence generated by translating the above-referenced coding sequence (cds) using the universal genetic code, i.e. the amino acid sequence also publically disclosed as

GenBank:
AY253715.1:
MGTVNKPVVGVLMGEGIITGTLRITNPVRASVLRYDDFHIDEDKLDTNSV
YEPYYHSDHAESSWVNRGESSRKAYDHNSPYIWPRNDYDGFLENAHEHHG
VYNQGRGIDSGERLMQPTQMSAQEDLGDDTGIHVIPTLNGDDRHKIVNVD
QRQYGDVFKGDLNPKPQGQRLIEVSVEENHPFTLRAPIQRIYGVRYTETW
SFLPSLTCTGDAAPAIQHICLKHTTCFQDVVVDVDCAENTKEDQLAEISY
RFQGKKEADQPWIVVNTSTLFDELELDPPEIEPGVLKVLRTEKQYLGVYI
WNMRGSDGTSTYATFLVTWKGDEKTRNPTPAVTPQPRGAEFHMWNYHSHV
FSVGDTFSLAMHLQYKIHEAPFDLLLEWLYVPIDPTCQPMRLYSTCLYHP
NAPQCLSHMNSGCTFTSPHLAQRVASTVYQNCEHADNYTAYCLGISHMEP
SFGLILHDGGTTLKFVDTPESLSGLYVFVVYFNGHVEAVAYTVVSTVDHF
VNAIEERGFPPTAGQPPATTKPKEITPVNPGTSPLLRYAAWTGGLAAVVL
LCLVIFLICTAKRMRVKAYRVDKSPYNQSMYYAGLPVDDFEDSESTDTEE
EFGNAIGGSHGGSSYTVYIDKTR

An exemplary Gly E amino acid sequence is GenBank accession number AAP32865.1—SEQ ID NO: 1. A most suitable GlyE amino acid sequence is SEQ ID NO: 2.

Suitably said Gly E antigen comprises SEQ ID NO: 1 or SEQ ID NO: 2.

Suitably said Gly E antigen consists of SEQ ID NO: 1 or SEQ ID NO: 2.

Suitably said Gly E antigen comprises, or consists of, full length Gly E antigen as shown in SEQ ID NO: 1 or SEQ ID NO: 2.

Suitably said Gly E antigen does not comprise any Truncations/Mutations/Tags/Linkers/Fusions compared to the sequence shown in SEQ ID NO: 1 or SEQ ID NO: 2.

bgh Polyadenylation Signal

The bovine growth hormone polyadenylation (bgh-PolyA) signal is a specialised termination sequence for protein expression in eukaryotic cells. This DNA sequence is optionally added to the nucleic acid sequence encoding the GlyE antigen.

An exemplary bgh polyadenylation signal has the sequence shown in SEQ ID NO: 6.

Suitably expression of the antigen is controlled by a standard promoter such as the ‘long CMV’ promoter. An exemplary sequence of the ‘long CMV’ promoter is shown in SEQ ID NO: 7.

Uses

Suitably the method is a method of immunising.

In one aspect, the invention relates to a composition comprising an adenoviral vector, said adenoviral vector comprising GlyE.

Suitably the composition does not comprise adjuvant. Adjuvant can cause reactogenicity, especially in primates such as humans. Thus it is an advantage that the composition of the invention is effective without adjuvant. Suitably adjuvant is omitted. Suitably the composition consists of elements other than adjuvant. Suitably adjuvant is specifically excluded from the compositions of the invention. Suitably the composition is an adjuvant-free composition.

The invention may be used in prevention of primary VZV infection which causes chickenpox in children and other susceptible individuals.

In another aspect, the invention relates to use of a composition as described above in medicine.

In another aspect, the invention relates to use of a composition as described above in the preparation of a medicament for VZV infection. Suitably said medicament is for controlling VZV infection. Suitably said medicament is for preventing resurgence of VZV infection. Suitably said medicament is for controlling shingles. Suitably said medicament is for preventing shingles.

In another aspect, the invention relates to a method for inducing an immune response in a subject, said method comprising administering to said subject a composition as described above.

Suitably the immune response comprises cell mediated immunity. Suitably the immune response comprises a T-cell response. Suitably the T-cell response comprises a CD4+ T-cell response.

In another aspect, the invention relates to a method comprising administering a first composition comprising an adenovirus based vector and a second composition comprising an adenovirus based vector.

In another aspect, the invention relates to a method comprising administering a first composition comprising a first adenovirus based vector and a second composition comprising a second adenovirus based vector.

Suitably the composition and the second composition are different.

Most suitably the first adenovirus based vector and the second adenovirus based vector are different.

Suitably said subject is a mammal.

Suitably said subject is a primate.

Suitably said subject is a human.

The invention also relates to use of a vector, composition or medicament as described herein for treatment of VZV infection.

The invention also relates to use of a vector, composition or medicament as described herein for control of VZV infection.

The invention also relates to use of a vector, composition or medicament as described herein for control of dormant VZV infection.

The invention also relates to use of a vector, composition or medicament as described herein for prevention of VZV infection.

The invention also relates to use of a vector, composition or medicament as described herein for prevention of resurgence of VZV infection.

Compositions

Suitably the composition is an antigenic composition.

Suitably the composition is an immunogenic composition.

Suitably the composition is a vaccine composition.

Suitably the composition is a pharmaceutical composition.

Suitably the composition is formulated for administration to mammals, suitably to primates, most suitably to humans.

Suitably the composition is formulated taking into account its route of administration.

Suitably the composition is formulated to be suitable for the route of administration specified. Suitably the composition is formulated to be suitable for the route of administration selected by the operator or physician.

It is an advantage of the invention that the compositions do not require adjuvant.

Suitably the compositions of the invention for administration advantageously do not comprise adjuvant. Suitably adjuvant is omitted from compositions of the invention.

Most suitably adjuvant is excluded from compositions of the invention. Most suitably the compositions of the invention are adjuvant-free.

Administration

In principle any suitable route of administration may be used.

Suitably said composition is administered by a route of administration selected from a group consisting of intranasal, oral, aerosol, subcutaneous, intradermal and intramuscular.

More suitably said composition is administered by a route of administration selected from a group consisting of subcutaneous, intradermal and intramuscular.

Most suitably said administration is intramuscular.

Suitably the composition of the invention is administered intramuscularly.

Suitably the composition of the invention is formulated for intramuscular administration.

Suitably the composition of the invention is given as a single dose.

Dose

It should be noted that there are alternate ways of describing the dose for adenoviral vectors.

Viral particles—vp/mL. This refers to the count of total viral particles administered.

Infectious units—i.u./mL. This refers to the number of infectious units administered, and can be correlated more accurately with immunogenicity.

By convention, clinical trials in the UK tend to provide the dose in terms of viral particles.

A typical range would be 1×10⁷ vp to 1×10¹¹ vp, or 1×10⁸ vp to 5×10¹¹ vp. More suitably a single dose is in the range of 5×10⁸ to 5×10¹⁰ viral particles per administration; more suitably in the range of 5×10⁹ to 5×10¹⁰ viral particles per administration; more suitably in the range of 2.5×10¹⁰ to 5×10¹⁰ viral particles per administration, for an adult human.

Most suitably the dose is, or is about, 2-5×10¹⁰ viral particles per administration for an adult human.

Child doses are suitably determined by a physician with reference to the guidance provided herein for adult doses. Exemplary child dose=½ an adult dose or 1×10¹⁰ vp/child.

Infectious units will depend on the P:I ratio (viral genome:infectivity particle ratio) for any given preparation as is known in the art.

Suitably no adjuvant is administered with the viral vector of the invention.

Suitably the viral vector of the invention is formulated with simple buffer. An exemplary buffer may be as shown below under the heading ‘Formulation’.

Suitably the composition is administered as a single dose.

As used herein, ‘adult’ means a subject of at least 18 years of age.

As used herein, ‘child’ means a subject of less than 18 years of age.

Suitably the composition of the invention may be administered to a subject aged 2 years or more, suitably 18 years or more, suitably 60 years or more, suitably 70 years or more, suitably 79 years or more.

Doses are typically determined by a physician taking into account factors such as age, weight, gender or other relevant considerations. Doses given herein are exemplary doses. Unless otherwise indicated, all doses are for ‘adult’ subjects—child doses may be determined from those e.g. a child dose may be 50% of an adult dose, or more suitably a child dose is as described herein.

Database Release

Sequences deposited in databases can change over time. Suitably the current version of sequence database(s) are relied upon. Alternatively, the release in force at the date of filing is relied upon.

As the skilled person knows, the accession numbers may be version/dated accession numbers. The citeable accession numbers for the current database entry are the same as above, but omitting the decimal point and any subsequent digits.

GenBank is the NIH genetic sequence database, an annotated collection of all publicly available DNA sequences (National Center for Biotechnology Information, U.S. National Library of Medicine 8600 Rockville Pike, Bethesda Md., 20894 USA; Nucleic Acids Research, 2013 January; 41(D1):D36-42) and accession numbers provided relate to this unless otherwise apparent. Suitably the GenBank database release referred to is 15 Dec. 2017, NCBI-GenBank Release 223.0.

UniProt (Universal Protein Resource) is a comprehensive catalogue of information on proteins (‘UniProt: a hub for protein information’ Nucleic Acids Res. 43: D204-D212 (2015).). For the avoidance of doubt, UniProt Release 2015_11 is relied upon.

In more detail, the UniProt consortium European Bioinformatics Institute (EBI), SIB Swiss Institute of Bioinformatics and Protein Information Resource (PIR)'s UniProt Knowledgebase (UniProtKB) Release 2018_01, (31 Jan. 2018) is relied upon.

Applications

The compositions of the invention may be used as a chicken pox vaccine, most suitably in children. Thus the invention relates to use of the compositions as described above to prevent chickenpox. In this aspect the composition is administered to infants and/or children and/or adults in at least one dose; suitably said administration is before exposure to generate a protective immune response.

The compositions of the invention may be used as vaccines in immune-compromised children. In the prior art, children are given the Zostavax™ at a lower dose than adults.

In the art it has occasionally been disclosed that adenovirus may be used as a prime followed by pox virus as a boost. However, according to the present invention it may be observed that applying the vaccine to a person having had previous exposure to VZV means that they may have an existing response (e.g. existing immune response against VZV). By “existing response” suitably is meant the individual has been previously pre-exposed to VZV; this will typically be assessed by measuring seroconversion against VZV surface antigens.

The U.K. national health service (NHS) describes it as follows: ‘you can have a blood test to check if you have antibodies to the disease, which proves you've had chickenpox before.’ https://www.nhs.uk/conditions/vaccinations/when-is-chickenpox-vaccine-needed/#how-to-check-if-youve-had-chickenpox-before Therefore, when a patient has had previous exposure to VZV, administration of the vaccine according to the invention may be regarded as a boost. Thus, in essence the invention may be considered as teaching the use of adenovirus vector as a boost which is a departure from the prior art which teaches that pox viral vectors are best for boosting.

The VZV from an earlier infection (e.g. having chickenpox as a child) can remain dormant in a patient's body, such as in the nervous system, and can re-emerge as shingles later in life. A ‘dormant’ VZV infection may be defined as one that is no longer causing an active infection.

In one embodiment the invention relates to a composition for administration to a mammal comprising an adenoviral vector as described above.

### FURTHER APPLICATIONS

In one aspect, the invention relates to a viral vector comprising nucleic acid having a polynucleotide sequence encoding at least one epitope of the varicella-zoster virus (VZV) Gly E antigen, wherein said viral vector is an adenoviral vector of human or simian origin.

In one aspect, the invention relates to a viral vector comprising nucleic acid having a polynucleotide sequence encoding at least one CD4 T cell and one CD8 T cell epitope of the varicella-zoster virus (VZV) Gly E antigen, wherein said viral vector is an adenoviral vector of human or simian origin.

In one aspect, the invention relates to use of a composition as described above in the preparation of a medicament that induces both CD4+ and CD8+ T cell responses to Gly E antigen in a vaccinated subject.

In one aspect, the invention relates to use of a composition as described above in the preparation of a medicament that induces both CD4+ and CD8+ T cell responses to Gly E antigen in a vaccinated subject, where that subject is a human.

In one aspect, the invention relates to use of a composition as described above in the preparation of a medicament that induces both CD4+ and CD8+ T cell responses and antibodies to Gly E antigen in a vaccinated subject, where that subject is a human.

### FURTHER ADVANTAGES

One focus of the invention is the provision of the gE antigen in the context of an adeno vector such as a ChAdOx vector.

Here we present data showing the effectiveness of the ChAdOx-gE construct. This has been compared to the existing Zostavax™, and we demonstrate that the construct of the invention is superior.

These superior results are better than might be expected based on the known properties of the ChAdOx vector and/or the information on the gE antigen in the art (which combination had never been disclosed in the art).

In particular we show that the inventors' constructs are surprisingly effective, and/or that prejudice in the art would have taught against using this construct, and/or that these constructs are obscure/cryptic/special.

Adenoviral vectors are typically used for priming immunisations in the art, whereas pox viral vectors are typically used for boosting applications. It can be observed that since most people already have some existing T cell response from past infection with Zoster virus (VZV), that we are in fact teaching use of adenoviral vectors for boosting in this invention. This has not been done before for Zoster virus (VZV). Thus in one embodiment the invention relates to a method for boosting pre-existing immune response(s) to VZV in a mammal, by administering a composition as described above to said mammal. In one embodiment the invention relates to a composition as described above for use in boosting pre-existing immune response(s) to VZV in a mammal. Whether or not a mammal possesses pre-existing immune response(s) to VZV may be determined by assessing seroconversion against VZV surface antigens as described above.

A key demonstration of the improvement delivered by the invention is based on the data such as efficacy data shown herein.

In this regard, the inventors are generating excellent T cell responses with the vector of the invention. The existing Zostavax™ vaccine has focused on the antibody response.

Thus the invention is a measurable improvement over the art.

There is the added advantage (which is not expected) that a single dose of an adenovirus as described herein gives as strong a humoral response as a protein and adjuvant. This is a measurable improvement over the art.

### BRIEF DESCRIPTION OF THE DRAWINGS

FIG. 1 shows a plot

FIG. 2 shows a bar chart

FIG. 3 shows a graph

FIG. 4 shows a plot

FIG. 5 shows a sequence alignment. “Insert” means SEQ ID NO: 4 (i.e. an exemplary nucleotide sequence encoding the antigen cassette). “AY253715.1” means SEQ ID NO: 3 (i.e. the wild type nucleotide sequence encoding the GlyE antigen).

FIG. 6 shows a sequence alignment (CLUSTAL 0(1.2.4) multiple sequence alignment). “vaccine” means SEQ ID NO: 2 (i.e. an exemplary VZV GlyE amino acid sequence antigen cassette). “AY253715.1” means SEQ ID NO: 1 (i.e. the wild type VZV GlyE amino acid sequence).

FIG. 7 shows a photograph

FIG. 8 shows a plot and a graph

FIG. 9 shows four graphs

FIG. 10 shows a graph

FIG. 11 shows a graph and two plots

FIG. 12 shows a bar chart

FIG. 13 shows a bar chart

FIG. 14 shows a plot

FIG. 15 shows a plot

### EXAMPLES

### Example 1—Viral Vectored Vaccines Against VZV

We have generated viral vectored vaccines toward VZV.

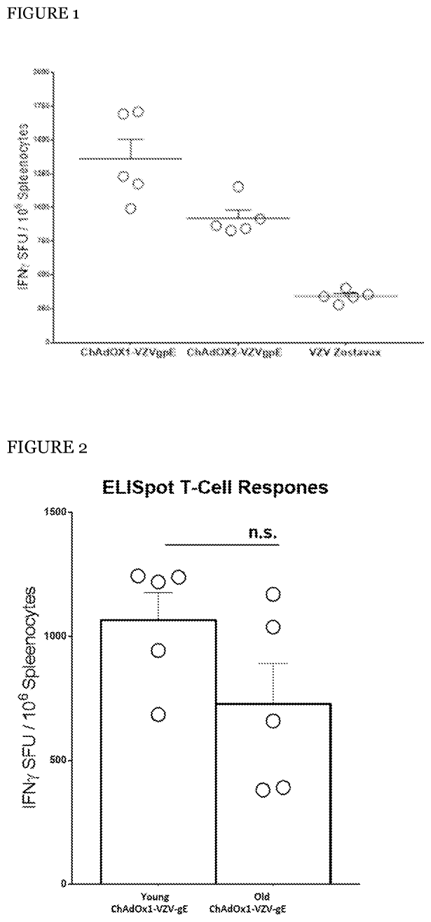

Our data suggest that the vaccine of the invention outperforms a currently licensed prior art Zoster vaccine as assessed for CMI pre-clinically (FIG. 1).

The higher CMI routinely achieved with viral vectored vaccines, when compared to other vaccine modalities, is likely to translate to higher efficacy, while advantageously only a single shot of viral vectored vaccine may be required for efficacy in contrast to repeated administration of known protein-in-adjuvant vaccines.

We refer to FIG. 1.

Groups of Balb/c mice (n=5) were vaccinated intramuscularly with 1×10⁷ IU of ChAdOx1-VZVgpE or 1×10⁷ IU of ChAdOx2-VZVgpE or 1.3×10³ pfu Zostax.

Splenocytes were collected 2 weeks after final vaccination and the cellular immune response against peptides spanning the whole glycoprotein-E were measured by ELISpot analysis.

Responses post ChAdOx1-VZV-gE were significantly higher than those post Zostavax.

This test is in young mice. Age was approx. >8 weeks.

It is noted that the prior art Zostavax vaccine can replicate in humans and without wishing to be bound by theory partial immunogenicity may be argued to have come from this. However, it has been demonstrated that non-replicating Zostavax vaccine is comparable in terms of measured immunogenicity to replicating Zostavax in man and can induce a similar immune response.

In any case, this is a fair test because none of the vaccines used replicate in mice.

Thus it is demonstrated that the invention outperforms prior art Zostavax.

We refer to FIG. 2.

8 wk⁺ old or aged ex-breeder female Balb/c mice were vaccinated intramuscularly with 1.00E+07 iu of ChAdOX1-VZV-gE Mice were culled approx. 2 weeks later and spleen ELISpot performed with peptides spanning the entire VZV gE insert.

Responses post ChAdOx1-VZV-gE were not significantly different.

This test is in aged mice.

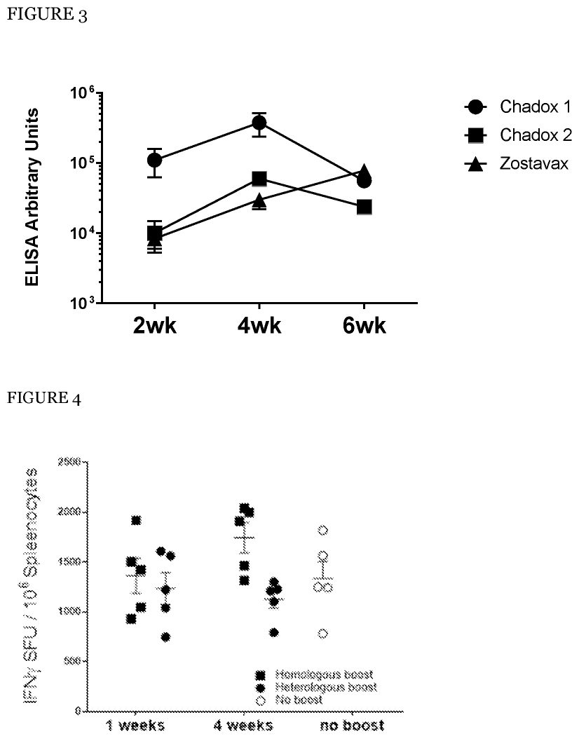

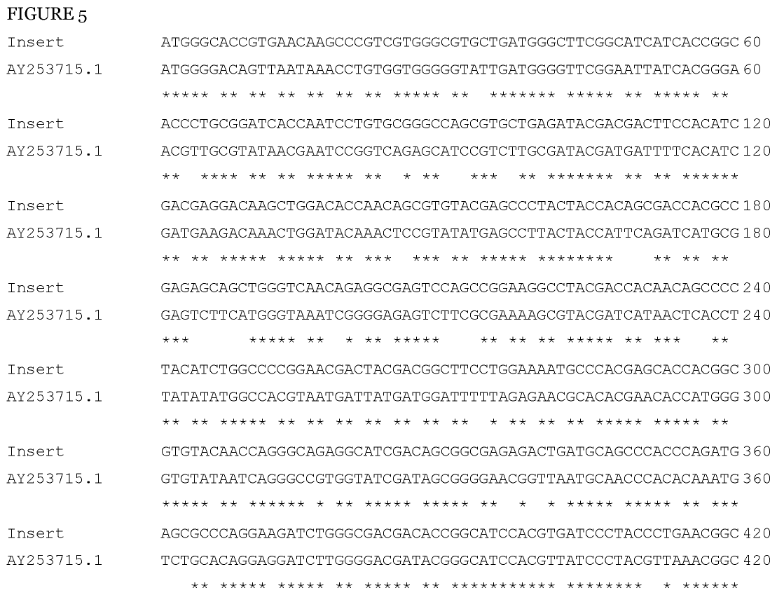

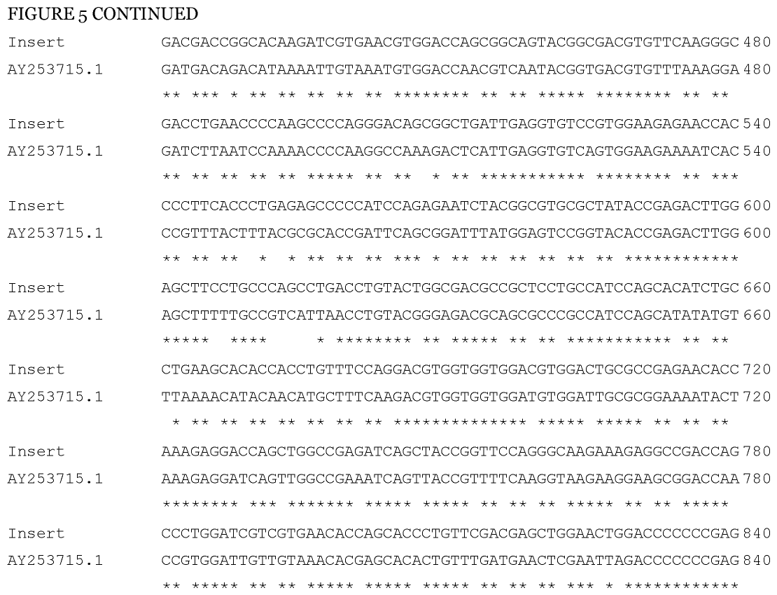

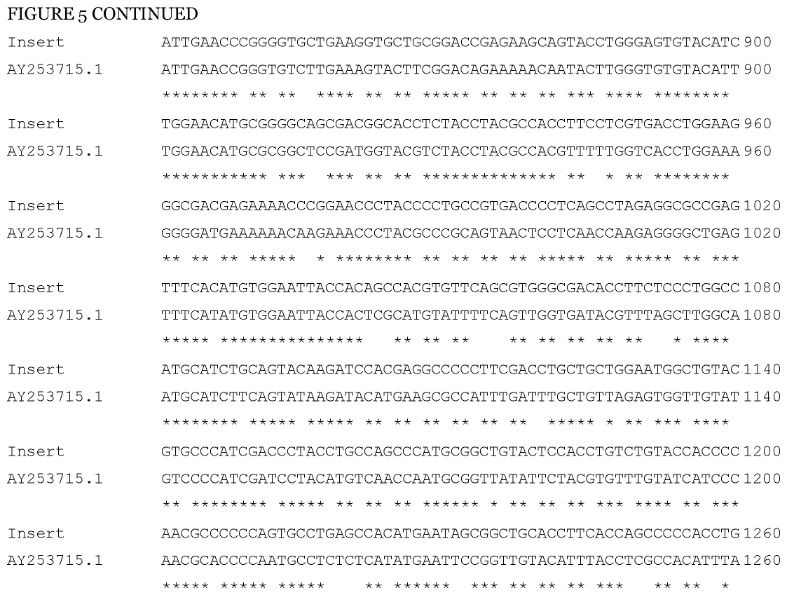

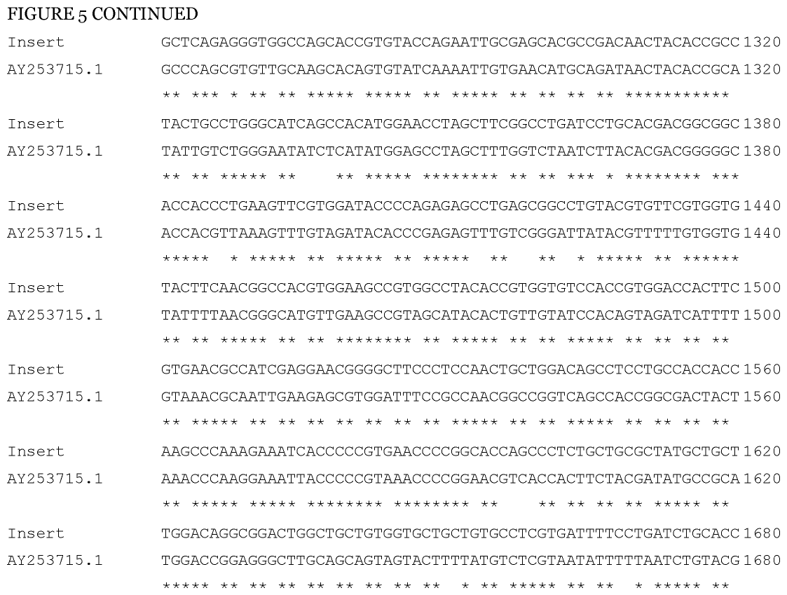

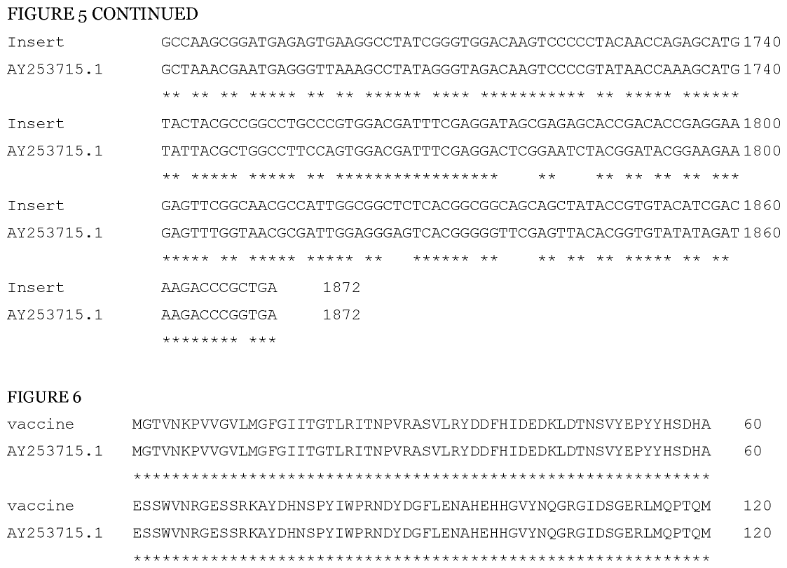

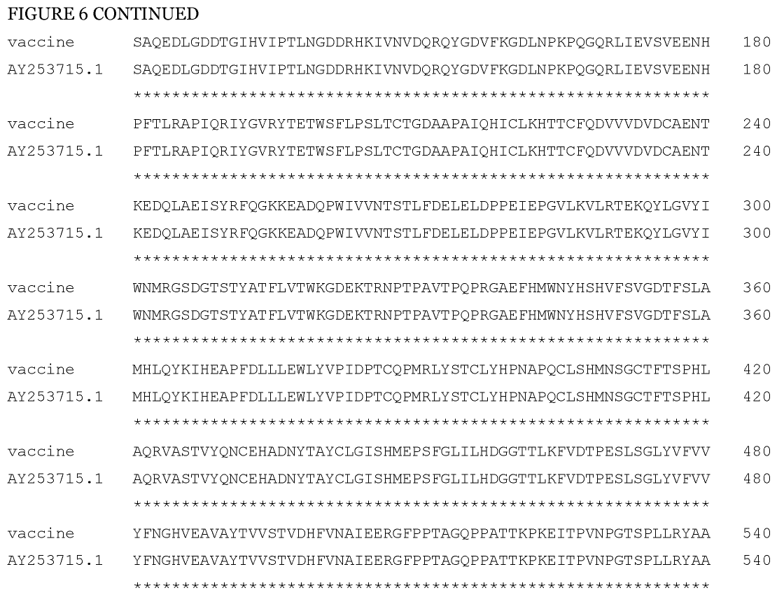

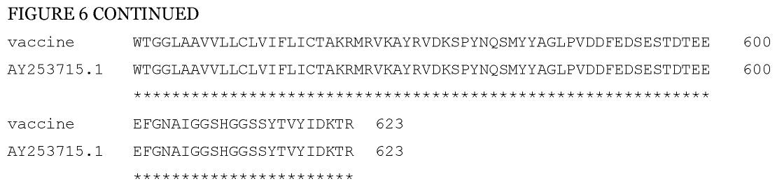

We refer to FIG. 3.

8 wk⁺ old female Balb/c mice were vaccinated intramuscularly with ChAdOX1-VZV-gE—group 1. 1.00E+07 iu ChAdOX1-VZV-gE then four weeks later boosted with 1.00E+07 iu ChAdOX1-VZV-gE ChAdOX2-VZV-gE—group 2. 1.00E+07 iu ChAdOX2-VZV-gE then four weeks later boosted with 1.00E+07 iu ChAdOX2-VZV-gE.

Zostavax group 3. 1.29E+03 VZV Zostavax then four weeks later boosted with 1.29E+03 VZV Zostavax. Sera was taken at the indicated timepoints and assayed for anti-VZV-gpE specific antibodies.

The inventors note that Kruskal-Wallis analysis shows with Dunn's multiple comparisons test significant difference in response between group 2 and 3, but not between group 1 and group 3 at 2 wk post-boost. This may represent a further advantage of this particular embodiment where the vector is ChAdOx1 i.e. the inventors would not have expected to see antibody response comparable to Zostavax (as evidenced by group 2—ChAdOx2) but group 1 (ChAdOx1 embodiment) generates a surprisingly good antibody response as well as good T cell responses.

The inventors note that there is no significant difference in responses in FIG. 4 after one shot antibody responses as 2 weeks or 16 weeks.

We refer to FIG. 4, 8 wk⁺ old female Balb/c mice were vaccinated intramuscularly with

lane 1. 1.00E+07 iu ChAdOX1-VZV-gE then one week later boosted with 1.00E+07 iu ChAdOX1-VZV-gE. (filled box)

lane 1. 1.00E+07 iu ChAdOX1-VZV-gE then one week later boosted with 1.00E+07 iu ChAdOX2-VZV-gE. (filled circle)

lane 2. 1.00E+07 iu ChAdOX1-VZV-gE then four weeks later boosted with 1.00E+07 iu ChAdOX1-VZV-gE (filled box)

lane 2. 1.00E+07 iu ChAdOX1-VZV-gE then four weeks later boosted with 1.00E+07 iu ChAdOX2-VZV-gE. (filled circle)

lane 3 (no Boost). 1.00E+07 iu ChAdOX1-VZV-gE (open circle)

Mice were culled approx. 2 weeks later and spleen ELISpot performed with peptides spanning the entire VZV gE insert.

Thus, lane 3 (no boost) represents a “one-shot” scheme; lanes 2 and 3 ‘filled boxes’ represent ‘homologous prime-boost’ schemes; lanes 2 and 3 ‘filled circle’ represent ‘heterologous prime-boost’ schemes. It could be argued that a heterologous second shot does not show augmentation—however, augmentation might be expected to show at a later time point—this is considered to be due to a response-curve effect.

Responses post ChAdOx1-VZV-gE were not significantly different across in young or aged animals for single-administration applications.

A preferred interval between prime and boost (in prime-boost applications; overall single-administration embodiments are preferred) is 4 weeks; when prime and boost are both Ad vectors, the interval may be for example 2, 4, 6 or 8 weeks.

Mouse Model System

Regarding the mouse model system for testing these vaccines, it should be noted that a 25 non-replicating zoster virus can give the same response as a replicating zoster virus in humans. Therefore, the mouse data presented herein do indeed represent a fair comparison since although the prior art Zostavax™ does induce a limited infection in humans which is important to boosting the immune response, neither the adenoviral vector constructs of the invention nor the Zostavax prior art comparator can replicate 30 in mice, and therefore the data provided in the application comparing those to formulations in mice are indeed fair and indicative of the superior properties of the vectors according to the invention.

### Example 2: Vectors of the Invention Express Efficiently

We present western blot analysis of viral vector expression of VZVgpE. Subconfluent HEK293T (ChAdOx 1) were infected with viruses with at the indicated MOI. Cells were harvested 18 h later, lysed and protein supernatant lysate run on a Biorad 4-12% gradient gel and probed with a 1:1000 dilution of abeam 52549 VZV in 0.05% PBST and expression detected with ECL reagent.

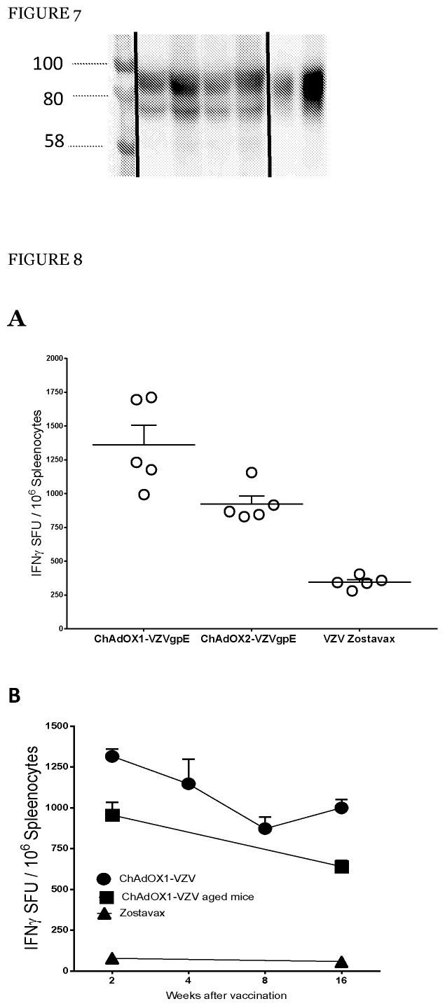

Results are shown in FIG. 7; lanes are as follows:
0—molecular markers
1—ChAdOx1 VZVgpE MOI 1 on HEK293T
2—ChAdOx1 VZVgpE MOI 5 on HEK293T
3—ChAdOx2 VZVgpE MOI 1 on HEK293T
4—ChAdOx2 VZVgpE MOI 5 on HEK293T
5—VZV+ve control ˜100 ng
6—VZV+ve control ˜500 ng

Thus it is demonstrated that the compositions of the invention produce expression of the antigen in human cells.

### Example 3: Immunogenicity of Viral Vectors Encoding Varicella Zoster Virus Glycoprotein E

We demonstrate cellular immunogenicity after one-shot vaccination against VZV. We refer to FIG. 8A. Groups of Balb/c mice (n=5) were vaccinated intramuscularly with 1×10⁷ IU of ChAdOx1-VZVgpE or 1×10⁷ IU of ChAdOx2-VZVgpE or 1.3×10³ pfu Zostax. Splenocytes were collected 2 weeks after final vaccination and the cellular immune response against peptides spanning the whole glycoprotein-E were measured by ELISpot analysis.

We refer to FIG. 8B. Groups of Balb/c mice (n=5, typically aged 8-10 weeks unless otherwise indicated) were vaccinated intramuscularly with 1×10⁷ IU of ChAdOx1-VZVgpE (‘aged mice’ are ex-breeders and typically >24 wk of age) or 1.3×10³ pfu Zostax. Splenocytes were collected at the times indicated after final vaccination and the cellular immune response against peptides spanning the whole glycoprotein-E were measured by ELISpot analysis.

These ELISpot data show that a cellular immune response, as demonstrated by the T cell response, is induced according to the invention. The response is evident at 2 weeks. The response is induced by a single administration. The response is induced by a single dose. The response is a sustained response as shown by the data at the 16 week timepoints.

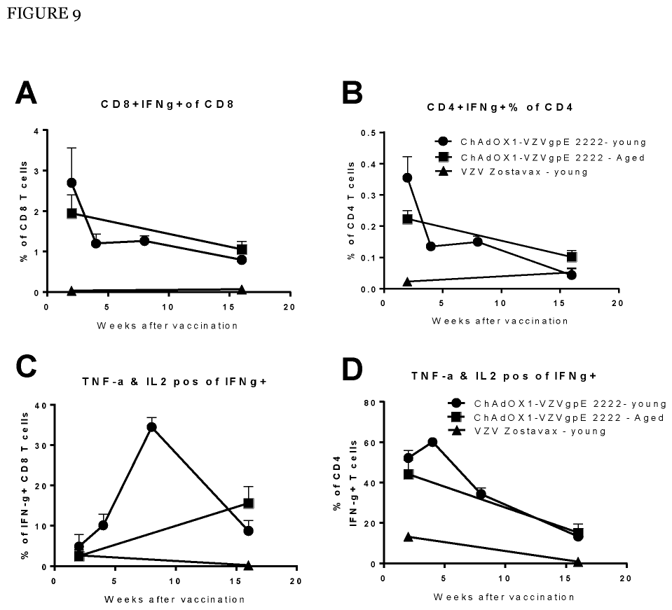

We refer to FIG. 9. The cellular immune response of the same groups of Balb/c mice as in FIG. 8 were assessed by Intracellular Cytokine Staining (ICS). As before (n=5, typically aged 8-10 weeks unless otherwise indicated) were vaccinated intramuscularly with 1×10⁷ IU of ChAdOx1-VZVgpE (‘aged mice’ are ex-breeders and typically >24 wk of age) or 1.3×10³ pfu Zostax. Splenocytes were collected at the times indicated after final vaccination and the cellular immune response toward epitopes spanning the whole glycoprotein-E were measured by ICS analysis.

FIG. 9A shows the percentage of CD8+ T cells secreting IFN-γ after stimulation with VZVgpE peptides and adjusted for background levels of secretion.

FIG. 9B shows the percentage of CD4+ T cells secreting IFN-γ after stimulation with VZVgpE peptides and adjusted for background levels of secretion.

FIG. 9C shows the percentage of IFN-γ+CD8+ T cells secreting TNFα and/or IL2 after stimulation with VZVgpE peptides and adjusted for background levels of secretion.

FIG. 9D shows the percentage of IFN-γ+CD4+ T cells secreting TNFα and/or IL2 after stimulation with VZVgpE peptides and adjusted for background levels of secretion.

Thus overall these FACS sorted experiments show that triple secreting CD4+ T cells (which are very good as without wishing to be bound by theory they are considered the most protective) are induced according to the invention. The data also show induction of CD8+ T cells, which are also very beneficial.

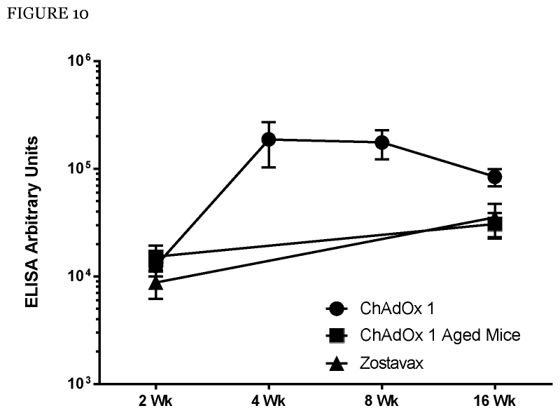

We refer to FIG. 10 which shows humoral immunity after one-shot vaccination against VZV. Groups of Balb/c mice (n=5, aged 8-10 weeks unless otherwise indicated) were vaccinated intramuscularly with 1×10⁷ IU of ChAdOx1-VZVgpE (‘aged mice’ are ex-breeders and typically >24 wk of age) or 1.3×10³ pfu Zostax. Sera were collected at the times indicated after final vaccination and the humoral immune response toward affinity purified glycoproteins of Varizella Zoster Virus (Strain Ellen) were measured by ELISA.

For clarity please note that FIGS. 8B, 9 and 10 show data from the same experiments.

It is a surprising benefit that the immunisations according to the present invention are also effective in raising/inducing antibody titers, despite the one-shot administration. It is a surprising benefit that the invention is as good as prior art compositions such as Zostavax for the induction of antibody responses.

### Example 4: Prime-Boost Study

We demonstrate immunogenicity after prime-boost vaccination against VZV.

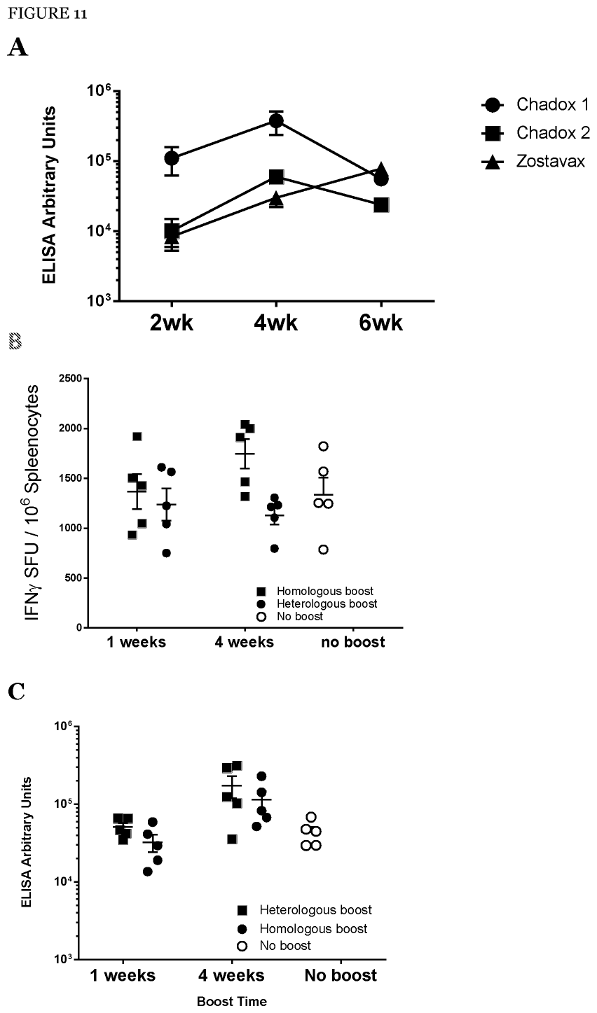

We refer to FIG. 11A.

8 wk⁺ old female Balb/c mice were vaccinated intramuscularly with ChAdOX1-VZV-gE—group 1. 1.00E+07 iu ChAdOX1-VZV-gE then four weeks later boosted with 1.00E+07 iu ChAdOX1-VZV-gE ChAdOX2-VZV-gE—group 2. 1.00E+07 iu ChAdOX2-VZV-gE then four weeks later boosted with 1.00E+07 iu ChAdOX2-VZV-gE.

Zostavax—group 3. 1.29E+03 VZV Zostavax then four weeks later boosted with 1.29E+03 VZV Zostavax. Sera was taken at the indicated timepoints and assayed for anti-VZV-gpE specific antibodies.

Referring to FIG. 11B & FIG. 11C, groups of Balb/c mice (n=5, aged 8-10 weeks unless otherwise indicated) were vaccinated intramuscularly with 1×10⁷ IU of ChAdOx1-VZVgpE and either not boosted (no boost) or boosted after 1 OR 4 weeks with 1×10⁷ IU of ChAdOx1-VZVgpE (homologous boost) or 1×10⁷ IU of ChAdOx2-VZVgpE (heterologous boost) a. Splenocytes were collected 2 weeks after final vaccination and the cellular immune response against peptides spanning the whole glycoprotein-E were measured by ELISpot analysis. b. Sera were collected 2 weeks after final vaccination and the humoral immune response toward affinity purified glycoproteins of Varizella Zoster Virus (Strain Ellen) were measured by ELISA.

These data show that the single-shot or single-administration embodiments of the invention provide as good a response as a prime-boost regime. Thus it is an advantage of the invention that only a single shot or single dose (single administration) is needed.

### Example 5: Comparative Study

The inventors compare the composition of the invention to prior art Shingrix™ across 3 doses and after one shot.

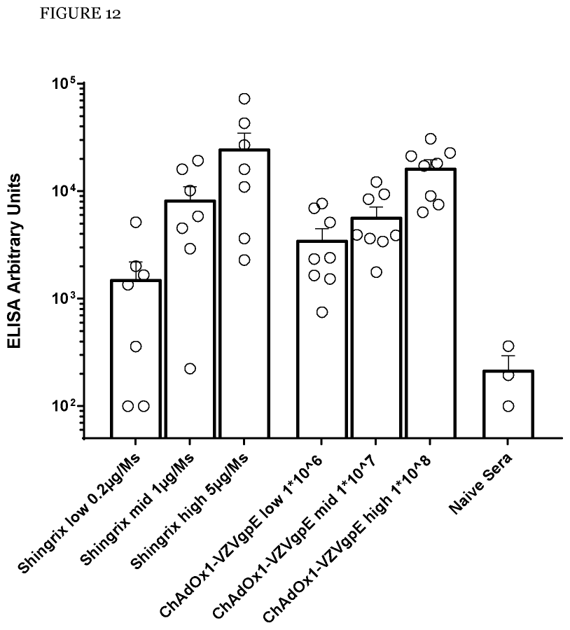

Humoral immunity after one-shot vaccination against VZV was tested. Groups of CD1 mice (n=7/8) were vaccinated intramuscularly with either ChAdOx1-VZVgpE or Shingrix™, at doses indicated (3 doses). Sera were collected at 4 weeks indicated after vaccination and the humoral immune response toward affinity purified glycoproteins of Varizella Zoster Virus (Strain Ellen) were measured by ELISA. We refer to FIG. 12. Mean with s.e.m. depicted.

| Vaccine Description | Group |
|---|---|
| Shingrix ™ low dose | 1 |
| 0.2 μg/mouse | |
| Shingrix ™ mid dose | 2 |
| 1 μg/mouse | |
| Shingrix ™ high dose | 3 |
| 5 μg/mouse | |
| ChAdOx1-VZVgpE low | 4 |
| 1*10\hat{ }6/mouse | |
| ChAdOx1-VZVgpE mid | 5 |
| 1*10\hat{ }7/mouse | |
| ChAdOx1-VZVgpE high | 6 |
| 1*10\hat{ }8/mouse | |

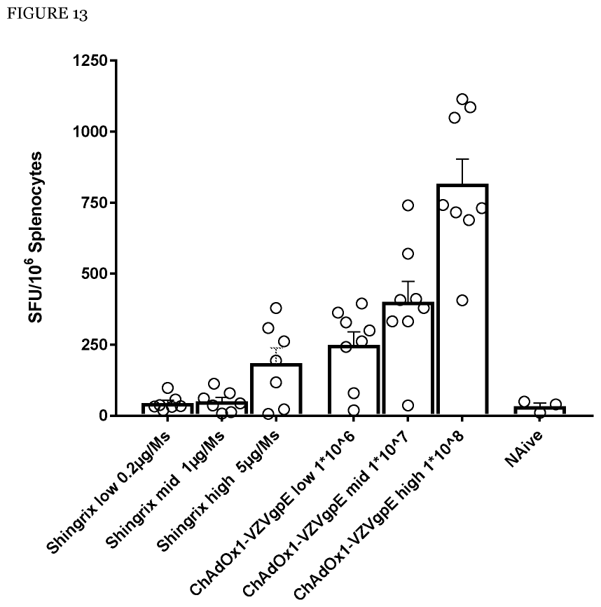

Cellular immunogenicity after one-shot vaccination against VZV was tested. Groups of CD1 mice (n=7/8) were vaccinated intramuscularly with either ChAdOx1-VZVgpE or Shingrix™, at doses indicated (3 doses). Splenocytes were collected 4 weeks after final vaccination and the cellular immune response against peptides spanning the whole glycoprotein-E were measured by ELISpot analysis. We refer to FIG. 13. Mean with s.e.m. depicted.

| Vaccine Description | Group |
|---|---|
| Shingrix ™ low dose | 1 |
| 0.2 μg/mouse | |
| Shingrix ™ mid dose | 2 |
| 1 μg/mouse | |
| Shingrix ™ high dose | 3 |
| 5 μg/mouse | |
| ChAdOx1-VZVgpE low | 4 |
| 1*10\hat{ }6/mouse | |
| ChAdOx1-VZVgpE mid | 5 |
| 1*10\hat{ }7/mouse | |
| ChAdOx1-VZVgpE high | 6 |
| 1*10\hat{ }8/mouse | |

### Example 6: Comparative Study

Groups of outbred CD-1 mice (n=8) were vaccinated intramuscularly with
Group 1; 1 ug of Shringrix with ASO1B adjuvant and four weeks later the animals were boosted with 1 ug of Shringrix with ASO1B adjuvant or
Group 2; no prime and four weeks later the animals were vaccinated with 1.3×10³ pfu Zostavax or
Group 3; 1.3×10³ pfu Zostavax and four weeks later animals were boosted with 1×10⁷ IU of ChAdOx1-VZVgpE or
Group 4; no prime and four weeks later the animals were vaccinated 1×10⁷ IU of ChAdOx1-VZVgpE or
Group 5; naïve animals.

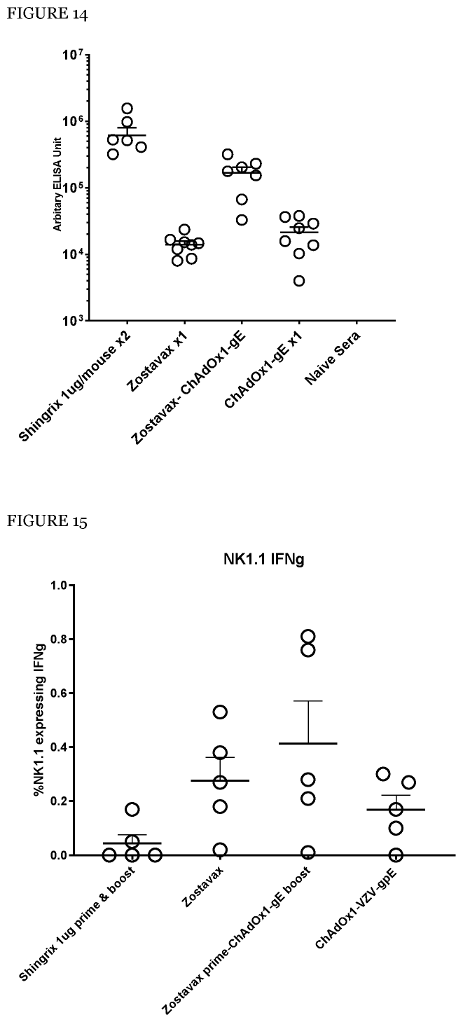

We refer to FIG. 14. The mean and standard error of the mean are depicted.

Serum was collected approximately three weeks after final vaccination and analysed for anti-VZVgpE antibodies.

Kruskal-Wallis analysis with Dunn's multiple comparison test demonstrates that Group 1; two shots of protein with adjuvant induces a significantly higher antibody titre when compared to Group 2; one shot of Zostavax or Group 4; one shot of ChAdOx1-VZVgpE, this result is as expected. However, there was no difference in the level of antibodies measured between Group 1 and Group 3. This is not expected, as ChAdOx1 after Zostavax would not be predicted to increase the humoral immune response to a comparable level of two shots of adjuvanted protein. This is an advantage, as currently UK adults aged 70 or over have been recommended to receive Zostavax vaccination, here we demonstrate that a boost vaccination of ChAdOx1-VZV-gpE can augment the antibody titres to those levels measured after two protein and adjuvant vaccinations, a regimen that is associated with efficacy of 91% or higher.

| Dunn's multiple | | | Adjusted P |
|---|---|---|---|
| comparisons test | Significant? | Summary | Value |
| Shingrix 1 ug/mouse x2 vs. | No | ns | >0.9999 |
| Zostavax-ChAdOx1-gE | | | |
| Shingrix 1 ug/mouse x2 vs. | Yes | * | 0.0172 |
| ChAdOx1-gE x1 | | | |
| Shingrix 1 ug/mouse x2 vs. | Yes | ** | 0.0019 |
| Zostavax x1 | | | |

### Example 7: Comparative Study

Groups of C57BL6 mice (n=5) were vaccinated intramuscularly with

Group 1; 1 ug of Shringrix with ASO1B adjuvant and four weeks later the animals were boosted with 1 ug of Shringrix with ASO1B adjuvant or

Group 2; no prime and four weeks later the animals were vaccinated with 1.3×10³ pfu Zostavax or

Group 3; 1.3×10³ pfu Zostavax and four weeks later animals were boosted with 1×10⁷ IU of ChAdOx1-VZVgpE or

Group 4; no prime and four weeks later the animals were vaccinated 1×10⁷ IU of ChAdOx1-VZVgpE.

We refer to FIG. 15. The mean and standard error of the mean are depicted.

Splenocytes were collected approximately four weeks after final vaccination and analysed for NK maturity and secretion of cytokines. Kruskal-Wallis analysis with Dunn's multiple comparison test demonstrates that NK cells after a prime-boost vaccination of 1.3×10³ pfu Zostax followed by 1×10⁷ IU of ChAdOx1-VZVgpE secrete more IFN-g (Group 3) when compared to the Shringix vaccination (Group 1). This is not expected and offers an advantage, NK cell activation has previously been demonstrated to augment the adaptive immune response and this strong induction of the innate immune response by ChAdOx1-VZVgpE after a Zostavax prime is not expected. Additionally, NK cells have been demonstrated to be critically important in mediating immunity against VZV infection (PMID; 2543925, 30565241).

Table of sequences

| | |
|---|---|
| SEQ ID NO: 1 | wild type VZV GlyE amino acid sequence |
| SEQ ID NO: 2 | exemplary VZV GlyE amino acid sequence |
| SEQ ID NO: 3 | wild type nucleotide sequence encoding |
| | VZV GlyE (Genbank accession number |
| | AY253715.1; this is the nucleotide |
| | sequence encoding the amino acid |
| | sequence of accession number |
| | AAP32865.1 - SEQ ID NO: 1: |
| | (AAP32865.1 glycoprotein E |
| | [Human alphaherpesvirus 3])) |
| SEQ ID NO: 4 | exemplary nucleotide sequence encoding |
| | VZV GlyE codon-optimised for humans |
| SEQ ID NO: 5 | ChAdOx2: Viral vector based on |
| | Chimpanzee adenovirus C68 |
| SEQ ID NO: 6 | bgh polyadenylation signal |
| SEQ ID NO: 7 | exemplary sequence of long CMV |
| | promoter |

Please note that ‘Gly E’ means glycoprotein E, and is sometimes referred to as ‘gE’.

In one embodiment Gly E sequence having similarity to antigen insert SEQ ID NO: 1 of 50% or less may be used. In one embodiment Gly E sequence having similarity to antigen insert SEQ ID NO: 1 of 50% or more may be used, suitably 60% or more, suitably 70% or more, suitably 80% or more, suitably 90% or more, suitably 95% or more.

SEQUENCE LISTING
SEQ ID NO: 1: exemplary Gly E sequence-GenBank accession number AAP32865.1-
(>A7P32865.1 glycoprotein E [Human alphaherpesvirus 3])(also
known as the amino acid sequence encoded by GenBank nucleotide
accession number AY253715.1):
MGTVNKPVVGVLMGFGIITGTLRITNPVRASVLRYDDFHIDEDKLDTNSVYEPYYHSDHAESSWVNRGESSRKAYDHN
SPYIWPRNDYDGFLENAHEHHGVYNQGRGIDSGERLMQPTQMSAQEDLGDDTGIHVIPTLNGDDRHKIVNVDQRQYGD
VFKGDLNPKPQGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWNMRGSDGTSTY
ATFLVTWKGDEKTRNPTPAVTPQPRGAEFHMWNYHSHVFSVGDTFSLAMHLQYKIHEAPFDLLLEWLYVPIDPTCQPM
RLYSTCLYHPNAPQCLSHMNSGCTFTSPHLAQRVASTVYQNCEHADNYTAYCLGISHMEPSFGLILHDGGTTLKFVDT
PESLSGLYVFVVYFNGHVEAVAYTVVSTVDHFVNAIEERGFPPTAGQPPATTKPKEITPVNPGTSPLLRYAAWTGGLA
AVVLLCLVIFLICTAKRMRVKAYRVDKSPYNQSMYYAGLPVDDFEDSESTDTEEEFGNAIGGSHGGSSYTVYIDKTR
SEQ ID NO: 2 exemplary VZV GlyE cassette amino acid sequence
(sometimes referred to as “Insert-protein”):
MGTVNKPVVGVLMGFGIITGTLRITNPVRASVLRYDDFHIDEDKLDTNSVYEPYYHSDHA
ESSWVNRGESSRKAYDHNSPYIWPRNDYDGFLENAHEHHGVYNQGRGIDSGERLMQPTQM
SAQEDLGDDTGIHVIPTLNGDDRHKIVNVDQRQYGDVFKGDLNPKPQGQRLIEVSVEENH
PFTLRAPIQRIYGVRYTETWSFLPSLTCTGDAAPAIQHICLKHTTCFQDVVVDVDCAENT
KEDQLAEISYRFQGKKEADQPWIVVNTSTLFDELELDPPEIEPGVLKVLRTEKQYLGVYI
WNMRGSDGTSTYATFLVTWKGDEKTRNPTPAVTPQPRGAEFHMWNYHSHVFSVGDTFSLA
MHLQYKIHEAPFDLLLEWLYVPIDPTCQPMRLYSTCLYHPNAPQCLSHMNSGCTFTSPHL
AQRVASTVYQNCEHADNYTAYCLGISHMEPSFGLILHDGGTTLKFVDTPESLSGLYVFVV
YFNGHVEAVAYTVVSTVDHFVNAIEERGFPPTAGQPPATTKPKEITPVNPGTSPLLRYAA
WTGGLAAVVLLCLVIFLICTAKRMRVKAYRVDKSPYNQSMYYAGLPVDDFEDSESTDTEE
EFGNAIGGSHGGSSYTVYIDKTR
SEQ ID NO: 3-wild type nucleotide sequence encoding VZV GlyE (Genbank accession
number AY253715.1;
this is the nucleotide sequence encoding the amino acid sequence
of accession number AAP32865.1-SEQ ID NO: 1:
(AAP32865.1 glycoprotein E [Human alphaherpesvirus 3])):
ATGGGGACAGTTAATAAACCTGTGGTGGGGGTATTGATGGGGTTCGGAATTATCACGGGAACGTTGCGTATAACGAAT
CCGGTCAGAGCATCCGTCTTGCGATACGATGATTTTCACATCGATGAAGACAAACTGGATACAAACTCCGTATATGAG
CCTTACTACCATTCAGATCATGCGGAGTCTTCATGGGTAAATCGGGGAGAGTCTTCGCGAAAAGCGTACGATCATAAC
TCACCTTATATATGGCCACGTAATGATTATGATGGATTTTTAGAGAACGCACACGAACACCATGGGGTGTATAATCAG
GGCCGTGGTATCGATAGCGGGGAACGGTTAATGCAACCCACACAAATGTCTGCACAGGAGGATCTTGGGGACGATACG
GGCATCCACGTTATCCCTACGTTAAACGGCGATGACAGACATAAAATTGTAAATGTGGACCAACGTCAATACGGTGAC
GTGTTTAAAGGAGATCTTAATCCAAAACCCCAAGGCCAAAGACTCATTGAGGTGTCAGTGGAAGAAAATCACCCGTTT
ACTTTACGCGCACCGATTCAGCGGATTTATGGAGTCCGGTACACCGAGACTTGGAGCTTTTTGCCGTCATTAACCTGT
ACGGGAGACGCAGCGCCCGCCATCCAGCATATATGTTTAAAACATACAACATGCTTTCAAGACGTGGTGGTGGATGTG
GATTGCGCGGAAAATACTAAAGAGGATCAGTTGGCCGAAATCAGTTACCGTTTTCAAGGTAAGAAGGAAGCGGACCAA
CCGTGGATTGTTGTAAACACGAGCACACTGTTTGATGAACTCGAATTAGACCCCCCCGAGATTGAACCGGGTGTCTTG
AAAGTACTTCGGACAGAAAAACAATACTTGGGTGTGTACATTTGGAACATGCGCGGCTCCGATGGTACGTCTACCTAC
GCCACGTTTTTGGTCACCTGGAAAGGGGATGAAAAAACAAGAAACCCTACGCCCGCAGTAACTCCTCAACCAAGAGGG
GCTGAGTTTCATATGTGGAATTACCACTCGCATGTATTTTCAGTTGGTGATACGTTTAGCTTGGCAATGCATCTTCAG
TATAAGATACATGAAGCGCCATTTGATTTGCTGTTAGAGTGGTTGTATGTCCCCATCGATCCTACATGTCAACCAATG
CGGTTATATTCTACGTGTTTGTATCATCCCAACGCACCCCAATGCCTCTCTCATATGAATTCCGGTTGTACATTTACC
TCGCCACATTTAGCCCAGCGTGTTGCAAGCACAGTGTATCAAAATTGTGAACATGCAGATAACTACACCGCATATTGT
CTGGGAATATCTCATATGGAGCCTAGCTTTGGTCTAATCTTACACGACGGGGGCACCACGTTAAAGTTTGTAGATACA
CCCGAGAGTTTGTCGGGATTATACGTTTTTGTGGTGTATTTTAACGGGCATGTTGAAGCCGTAGCATACACTGTTGTA
TCCACAGTAGATCATTTTGTAAACGCAATTGAAGAGCGTGGATTTCCGCCAACGGCCGGTCAGCCACCGGCGACTACT
AAACCCAAGGAAATTACCCCCGTAAACCCCGGAACGTCACCACTTCTACGATATGCCGCATGGACCGGAGGGCTTGCA
GCAGTAGTACTTTTATGTCTCGTAATATTTTTAATCTGTACGGCTAAACGAATGAGGGTTAAAGCCTATAGGGTAGAC
AAGTCCCCGTATAACCAAAGCATGTATTACGCTGGCCTTCCAGTGGACGATTTCGAGGACTCGGAATCTACGGATACG
GAAGAAGAGTTTGGTAACGCGATTGGAGGGAGTCACGGGGGTTCGAGTTACACGGTGTATATAGATAAGACCCGGTGA
SEQ ID NO: 4-exemplary nucleotide sequence encoding VZV GlyE antigen
cassette/expression cassette
(sometimes referred to as “>Insert-Vaccine”)
ATGGGCACCGTGAACAAGCCCGTCGTGGGCGTGCTGATGGGCTTCGGCATCATCACCGGCACCCTGCGGATCACCAAT
CCTGTGCGGGCCAGCGTGCTGAGATACGACGACTTCCACATCGACGAGGACAAGCTGGACACCAACAGCGTGTACGAG
CCCTACTACCACAGCGACCACGCCGAGAGCAGCTGGGTCAACAGAGGCGAGTCCAGCCGGAAGGCCTACGACCACAAC
AGCCCCTACATCTGGCCCCGGAACGACTACGACGGCTTCCTGGAAAATGCCCACGAGCACCACGGCGTGTACAACCAG
GGCAGAGGCATCGACAGCGGCGAGAGACTGATGCAGCCCACCCAGATGAGCGCCCAGGAAGATCTGGGCGACGACACC
GGCATCCACGTGATCCCTACCCTGAACGGCGACGACCGGCACAAGATCGTGAACGTGGACCAGCGGCAGTACGGCGAC
GTGTTCAAGGGCGACCTGAACCCCAAGCCCCAGGGACAGCGGCTGATTGAGGTGTCCGTGGAAGAGAACCACCCCTTC
ACCCTGAGAGCCCCCATCCAGAGAATCTACGGCGTGCGCTATACCGAGACTTGGAGCTTCCTGCCCAGCCTGACCTGT
ACTGGCGACGCCGCTCCTGCCATCCAGCACATCTGCCTGAAGCACACCACCTGTTTCCAGGACGTGGTGGTGGACGTG
GACTGCGCCGAGAACACCAAAGAGGACCAGCTGGCCGAGATCAGCTACCGGTTCCAGGGCAAGAAAGAGGCCGACCAG
CCCTGGATCGTCGTGAACACCAGCACCCTGTTCGACGAGCTGGAACTGGACCCCCCCGAGATTGAACCCGGGGTGCTG
AAGGTGCTGCGGACCGAGAAGCAGTACCTGGGAGTGTACATCTGGAACATGCGGGGCAGCGACGGCACCTCTACCTAC
GCCACCTTCCTCGTGACCTGGAAGGGCGACGAGAAAACCCGGAACCCTACCCCTGCCGTGACCCCTCAGCCTAGAGGC
GCCGAGTTTCACATGTGGAATTACCACAGCCACGTGTTCAGCGTGGGCGACACCTTCTCCCTGGCCATGCATCTGCAG
TACAAGATCCACGAGGCCCCCTTCGACCTGCTGCTGGAATGGCTGTACGTGCCCATCGACCCTACCTGCCAGCCCATG
CGGCTGTACTCCACCTGTCTGTACCACCCCAACGCCCCCCAGTGCCTGAGCCACATGAATAGCGGCTGCACCTTCACC
AGCCCCCACCTGGCTCAGAGGGTGGCCAGCACCGTGTACCAGAATTGCGAGCACGCCGACAACTACACCGCCTACTGC
CTGGGCATCAGCCACATGGAACCTAGCTTCGGCCTGATCCTGCACGACGGCGGCACCACCCTGAAGTTCGTGGATACC
CCAGAGAGCCTGAGCGGCCTGTACGTGTTCGTGGTGTACTTCAACGGCCACGTGGAAGCCGTGGCCTACACCGTGGTG
TCCACCGTGGACCACTTCGTGAACGCCATCGAGGAACGGGGCTTCCCTCCAACTGCTGGACAGCCTCCTGCCACCACC
AAGCCCAAAGAAATCACCCCCGTGAACCCCGGCACCAGCCCTCTGCTGCGCTATGCTGCTTGGACAGGCGGACTGGCT
GCTGTGGTGCTGCTGTGCCTCGTGATTTTCCTGATCTGCACCGCCAAGCGGATGAGAGTGAAGGCCTATCGGGTGGAC
AAGTCCCCCTACAACCAGAGCATGTACTACGCCGGCCTGCCCGTGGACGATTTCGAGGATAGCGAGAGCACCGACACC
GAGGAAGAGTTCGGCAACGCCATTGGCGGCTCTCACGGCGGCAGCAGCTATACCGTGTACATCGACAAGACCCGCTGA
SEQ ID NO: 5
ChAd0x2: Viral vector based on Chimpanzee adenovirus C68
ccatcttcaa taatatacct caaacttttt gtgcgcgtta atatgcaaat gaggcgtttg 60
aatttgggga ggaagggcgg tgattggtcg agggatgagc gaccgttagg ggcggggcga 120
gtgacgtttt gatgacgtgg ttgcgaggag gagccagttt gcaagttctc gtgggaaaag 180
tgacgtcaaa cgaggtgtgg tttgaacacg gaaatactca attttcccgc gctctctgac 240
aggaaatgag gtgtttctgg gcggatgcaa gtgaaaacgg gccattttcg cgcgaaaact 300
gaatgaggaa gtgaaaatct gagtaatttc gcgtttatgg cagggaggag tatttgccga 360
gggccgagta gactttgacc gattacgtgg gggtttcgat taccgtgttt ttcacctaaa 420
tttccgcgta cggtgtcaaa gtccggtgtt tttacgcgat cgctagcgac atcgatcaca 480
agtttgtaca aaaaagctga acgagaaacg taaaatgata taaatatcaa tatattaaat 540
tagattttgc ataaaaaaca gactacataa tactgtaaaa cacaacatat ccagtcacta 600
tggcggccgc cgatttattc aacaaagcca cgttgtgtct caaaatctct gatgttacat 660
tgcacaagat aaaaatatat catcatgaac aataaaactg tctgcttaca taaacagtaa 720
tacaaggggt gttatgagcc atattcaacg ggaaacgtct tgctcgaggc cgcgattaaa 780
ttccaacatg gatgctgatt tatatgggta taaatgggct cgtgataatg tcgggcaatc 840
aggtgcgaca atctatcgat tgtatgggaa gcccgatgcg ccagagttgt ttctgaaaca 900
tggcaaaggt agcgttgcca atgatgttac agatgagatg gtcagactaa actggctgac 960
ggaatttatg cctcttccga ccatcaagca ttttatccgt actcctgatg atgcatggtt 1020
actcaccact gcgatccccg ggaaaacagc attccaggta ttagaagaat atcctgattc 1080
aggtgaaaat attgttgatg cgctggcagt gttcctgcgc cggttgcatt cgattcctgt 1140
ttgtaattgt ccttttaaca gcgatcgcgt atttcgtctc gctcaggcgc aatcacgaat 1200
gaataacggt ttggttgatg cgagtgattt tgatgacgag cgtaatggct ggcctgttga 1260
acaagtctgg aaagaaatgc ataagctttt gccattctca ccggattcag tcgtcactca 1320
tggtgatttc tcacttgata accttatttt tgacgagggg aaattaatag gttgtattga 1380
tgttggacga gtcggaatcg cagaccgata ccaggatctt gccatcctat ggaactgcct 1440
cggtgagttt tctccttcat tacagaaacg gctttttcaa aaatatggta ttgataatcc 1500
tgatatgaat aaattgcagt ttcatttgat gctcgatgag tttttctaat cagaattggt 1560
taattggttg taacactggc acgcgtggat ccggcttact aaaagccaga taacagtatg 1620
cgtatttgcg cgctgatttt tgcggtataa gaatatatac tgatatgtat acccgaagta 1680
tgtcaaaaag aggtatgcta tgaagcagcg tattacagtg acagttgaca gcgacagcta 1740
tcagttgctc aaggcatata tgatgtcaat atctccggtc tggtaagcac aaccatgcag 1800
aatgaagccc gtcgtctgcg tgccgaacgc tggaaagcgg aaaatcagga agggatggct 1860
gaggtcgccc ggtttattga aatgaacggc tcttttgctg acgagaacag gggctggtga 1920
aatgcagttt aaggtttaca cctataaaag agagagccgt tatcgtctgt ttgtggatgt 1980
acagagtgat attattgaca cgcccgggcg acggatggtg atccccctgg ccagtgcacg 2040
tctgctgtca gataaagtct cccgtgaact ttacccggtg gtgcatatcg gggatgaaag 2100
ctggcgcatg atgaccaccg atatggccag tgtgccggtc tccgttatcg gggaagaagt 2160
ggctgatctc agccaccgcg aaaatgacat caaaaacgcc attaacctga tgttctgggg 2220
aatataaatg tcaggctccc ttatacacag ccagtctgca ggtcgaccat agtgactgga 2280
tatgttgtgt tttacagtat tatgtagtct gttttttatg caaaatctaa tttaatatat 2340
tgatatttat atcattttac gtttctcgtt cagctttctt gtacaaagtg gtgatcgatt 2400
cgacagatcg cgatcgcaag tgagtagtgt tctggggcgg gggaggacct gcatgagggc 2460
cagaataact gaaatctgtg cttttctgtg tgttgcagca gcatgagcgg aagcggctcc 2520
tttgagggag gggtattcag cccttatctg acggggcgtc tcccctcctg ggcgggagtg 2580
cgtcagaatg tgatgggatc cacggtggac ggccggcccg tgcagcccgc gaactcttca 2640
accctgacct atgcaaccct gagctcttcg tcgttggacg cagctgccgc cgcagctgct 2700
gcatctgccg ccagcgccgt gcgcggaatg gccatgggcg ccggctacta cggcactctg 2760
gtggccaact cgagttccac caataatccc gccagcctga acgaggagaa gctgttgctg 2820
ctgatggccc agctcgaggc cttgacccag cgcctgggcg agctgaccca gcaggtggct 2880
cagctgcagg agcagacgcg ggccgcggtt gccacggtga aatccaaata aaaaatgaat 2940
caataaataa acggagacgg ttgttgattt taacacagag tctgaatctt tatttgattt 3000
ttcgcgcgcg gtaggccctg gaccaccggt ctcgatcatt gagcacccgg tggatctttt 3060
ccaggacccg gtagaggtgg gcttggatgt tgaggtacat gggcatgagc ccgtcccggg 3120
ggtggaggta gctccattgc agggcctcgt gctcgggggt ggtgttgtaa atcacccagt 3180
catagcaggg gcgcagggca tggtgttgca caatatcttt gaggaggaga ctgatggcca 3240
cgggcagccc tttggtgtag gtgtttacaa atctgttgag ctgggaggga tgcatgcggg 3300
gggagatgag gtgcatcttg gcctggatct tgagattggc gatgttaccg cccagatccc 3360
gcctggggtt catgttgtgc aggaccacca gcacggtgta tccggtgcac ttggggaatt 3420
tatcatgcaa cttggaaggg aaggcgtgaa agaatttggc gacgcctttg tgcccgccca 3480
ggttttccat gcactcatcc atgatgatgg cgatgggccc gtgggcggcg gcctgggcaa 3540
agacgtttcg ggggtcggac acatcatagt tgtggtcctg ggtgaggtca tcataggcca 3600
ttttaatgaa tttggggcgg agggtgccgg actgggggac aaaggtaccc tcgatcccgg 3660
gggcgtagtt cccctcacag atctgcatct cccaggcttt gagctcggag ggggggatca 3720
tgtccacctg cggggcgata aagaacacgg tttccggggc gggggagatg agctgggccg 3780
aaagcaagtt ccggagcagc tgggacttgc cgcagccggt ggggccgtag atgaccccga 3840
tgaccggctg caggtggtag ttgagggaga gacagctgcc gtcctcccgg aggagggggg 3900
ccacctcgtt catcatctcg cgcacgtgca tgttctcgcg caccagttcc gccaggaggc 3960
gctctccccc cagggatagg agctcctgga gcgaggcgaa gtttttcagc ggcttgagtc 4020
cgtcggccat gggcattttg gagagggttt gttgcaagag ttccaggcgg tcccagagct 4080
cggtgatgtg ctctacggca tctcgatcca gcagacctcc tcgtttcgcg ggttgggacg 4140
gctgcgggag tagggcacca gacgatgggc gtccagcgca gccagggtcc ggtccttcca 4200
gggtcgcagc gtccgcgtca gggtggtctc cgtcacggtg aaggggtgcg cgccgggctg 4260
ggcgcttgcg agggtgcgct tcaggctcat ccggctggtc gaaaaccgct cccgatcggc 4320
gccctgcgcg tcggccaggt agcaattgac catgagttcg tagttgagcg cctcggccgc 4380
gtggcctttg gcgcggagct tacctttgga agtctgcccg caggcgggac agaggaggga 4440
cttgagggcg tagagcttgg gggcgaggaa gacggactcg ggggcgtagg cgtccgcgcc 4500
gcagtgggcg cagacggtct cgcactccac gagccaggtg aggtcgggct ggtcggggtc 4560
aaaaaccagt ttcccgccgt tctttttgat gcgtttctta cctttggtct ccatgagctc 4620
gtgtccccgc tgggtgacaa agaggctgtc cgtgtccccg tagaccgact ttatgggccg 4680
gtcctcgagc ggtgtgccgc ggtcctcctc gtagaggaac cccgcccact ccgagacgaa 4740
agcccgggtc caggccagca cgaaggaggc cacgtgggac gggtagcggt cgttgtccac 4800
cagcgggtcc accttttcca gggtatgcaa acacatgtcc ccctcgtcca catccaggaa 4860
ggtgattggc ttgtaagtgt aggccacgtg accgggggtc ccggccgggg gggtataaaa 4920
gggtgcgggt ccctgctcgt cctcactgtc ttccggatcg ctgtccagga gcgccagctg 4980
ttggggtagg tattccctct cgaaggcggg catgacctcg gcactcaggt tgtcagtttc 5040
tagaaacgag gaggatttga tattgacggt gccggcggag atgcctttca agagcccctc 5100
gtccatctgg tcagaaaaga cgatcttttt gttgtcgagc ttggtggcga aggagccgta 5160
gagggcgttg gagaggagct tggcgatgga gcgcatggtc tggttttttt ccttgtcggc 5220
gcgctccttg gcggcgatgt tgagctgcac gtactcgcgc gccacgcact tccattcggg 5280
gaagacggtg gtcagctcgt cgggcacgat tctgacctgc cagccccgat tatgcagggt 5340
gatgaggtcc acactggtgg ccacctcgcc gcgcaggggc tcattagtcc agcagaggcg 5400
tccgcccttg cgcgagcaga aggggggcag ggggtccagc atgacctcgt cgggggggtc 5460
ggcatcgatg gtgaagatgc cgggcaggag gtcggggtca aagtagctga tggaagtggc 5520
cagatcgtcc agggcagctt gccattcgcg cacggccagc gcgcgctcgt agggactgag 5580
gggcgtgccc cagggcatgg gatgggtaag cgcggaggcg tacatgccgc agatgtcgta 5640
gacgtagagg ggctcctcga ggatgccgat gtaggtgggg tagcagcgcc ccccgcggat 5700
gctggcgcgc acgtagtcat acagctcgtg cgagggggcg aggagccccg ggcccaggtt 5760
ggtgcgactg ggcttttcgg cgcggtagac gatctggcgg aaaatggcat gcgagttgga 5820
ggagatggtg ggcctttgga agatgttgaa gtgggcgtgg ggcagtccga ccgagtcgcg 5880
gatgaagtgg gcgtaggagt cttgcagctt ggcgacgagc tcggcggtga ctaggacgtc 5940
cagagcgcag tagtcgaggg tctcctggat gatgtcatac ttgagctgtc ccttttgttt 6000
ccacagctcg cggttgagaa ggaactcttc gcggtccttc cagtactctt cgagggggaa 6060
cccgtcctga tctgcacggt aagagcctag catgtagaac tggttgacgg ccttgtaggc 6120
gcagcagccc ttctccacgg ggagggcgta ggcctgggcg gccttgcgca gggaggtgtg 6180
cgtgagggcg aaagtgtccc tgaccatgac cttgaggaac tggtgcttga agtcgatatc 6240
gtcgcagccc ccctgctccc agagctggaa gtccgtgcgc ttcttgtagg cggggttggg 6300
caaagcgaaa gtaacatcgt tgaagaggat cttgcccgcg cggggcataa agttgcgagt 6360
gatgcggaaa ggttggggca cctcggcccg gttgttgatg acctgggcgg cgagcacgat 6420
ctcgtcgaag ccgttgatgt tgtggcccac gatgtagagt tccacgaatc gcggacggcc 6480
cttgacgtgg ggcagtttct tgagctcctc gtaggtgagc tcgtcggggt cgctgagccc 6540
gtgctgctcg agcgcccagt cggcgagatg ggggttggcg cggaggaagg aagtccagag 6600
atccacggcc agggcggttt gcagacggtc ccggtactga cggaactgct gcccgacggc 6660
cattttttcg ggggtgacgc agtagaaggt gcgggggtcc ccgtgccagc gatcccattt 6720
gagctggagg gcgagatcga gggcgagctc gacgagccgg tcgtccccgg agagtttcat 6780
gaccagcatg aaggggacga gctgcttgcc gaaggacccc atccaggtgt aggtttccac 6840
atcgtaggtg aggaagagcc tttcggtgcg aggatgcgag ccgatgggga agaactggat 6900
ctcctgccac caattggagg aatggctgtt gatgtgatgg aagtagaaat gccgacggcg 6960
cgccgaacac tcgtgcttgt gtttatacaa gcggccacag tgctcgcaac gctgcacggg 7020
atgcacgtgc tgcacgagct gtacctgagt tcctttgacg aggaatttca gtgggaagtg 7080
gagtcgtggc gcctgcatct cgtgctgtac tacgtcgtgg tggtcggcct ggccctcttc 7140
tgcctcgatg gtggtcatgc tgacgagccc gcgcgggagg caggtccaga cctcggcgcg 7200
agcgggtcgg agagcgagga cgagggcgcg caggccggag ctgtccaggg tcctgagacg 7260
ctgcggagtc aggtcagtgg gcagcggcgg cgcgcggttg acttgcagga gtttttccag 7320
ggcgcgcggg aggtccagat ggtacttgat ctccaccgcg ccattggtgg cgacgtcgat 7380
ggcttgcagg gtcccgtgcc cctggggtgt gaccaccgtc ccccgtttct tcttgggcgg 7440
ctggggcgac gggggcggtg cctcttccat ggttagaagc ggcggcgagg acgcgcgccg 7500
ggcggcaggg gcggctcggg gcccggaggc aggggcggca ggggcacgtc ggcgccgcgc 7560
gcgggtaggt tctggtactg cgcccggaga agactggcgt gagcgacgac gcgacggttg 7620
acgtcctgga tctgacgcct ctgggtgaag gccacgggac ccgtgagttt gaacctgaaa 7680
gagagttcga cagaatcaat ctcggtatcg ttgacggcgg cctgccgcag gatctcttgc 7740
acgtcgcccg agttgtcctg gtaggcgatc tcggtcatga actgctcgat ctcctcctct 7800
tgaaggtctc cgcggccggc gcgctccacg gtggccgcga ggtcgttgga gatgcggccc 7860
atgagctgcg agaaggcgtt catgcccgcc tcgttccaga cgcggctgta gaccacgacg 7920
ccctcgggat cgccggcgcg catgaccacc tgggcgaggt tgagctccac gtggcgcgtg 7980
aagaccgcgt agttgcagag gcgctggtag aggtagttga gcgtggtggc gatgtgctcg 8040
gtgacgaaga aatacatgat ccagcggcgg agcggcatct cgctgacgtc gcccagcgcc 8100
tccaaacgtt ccatggcctc gtaaaagtcc acggcgaagt tgaaaaactg ggagttgcgc 8160
gccgagacgg tcaactcctc ctccagaaga cggatgagct cggcgatggt ggcgcgcacc 8220
tcgcgctcga aggcccccgg gagttcctcc acttcctctt cttcctcctc cactaacatc 8280
tcttctactt cctcctcagg cggcagtggt ggcgggggag ggggcctgcg tcgccggcgg 8340
cgcacgggca gacggtcgat gaagcgctcg atggtctcgc cgcgccggcg tcgcatggtc 8400
tcggtgacgg cgcgcccgtc ctcgcggggc cgcagcgtga agacgccgcc gcgcatctcc 8460
aggtggccgg gggggtcccc gttgggcagg gagagggcgc tgacgatgca tcttatcaat 8520
tgccccgtag ggactccgcg caaggacctg agcgtctcga gatccacggg atctgaaaac 8580
cgctgaacga aggcttcgag ccagtcgcag tcgcaaggta ggctgagcac ggtttcttct 8640
ggcgggtcat gttggttggg agcggggcgg gcgatgctgc tggtgatgaa gttgaaatag 8700
gcggttctga gacggcggat ggtggcgagg agcaccaggt ctttgggccc ggcttgctgg 8760
atgcgcagac ggtcggccat gccccaggcg tggtcctgac acctggccag gtccttgtag 8820
tagtcctgca tgagccgctc cacgggcacc tcctcctcgc ccgcgcggcc gtgcatgcgc 8880
gtgagcccga agccgcgctg gggctggacg agcgccaggt cggcgacgac gcgctcggcg 8940
aggatggctt gctggatctg ggtgagggtg gtctggaagt catcaaagtc gacgaagcgg 9000
tggtaggctc cggtgttgat ggtgtaggag cagttggcca tgacggacca gttgacggtc 9060
tggtggcccg gacgcacgag ctcgtggtac ttgaggcgcg agtaggcgcg cgtgtcgaag 9120
atgtagtcgt tgcaggtgcg caccaggtac tggtagccga tgaggaagtg cggcggcggc 9180
tggcggtaga gcggccatcg ctcggtggcg ggggcgccgg gcgcgaggtc ctcgagcatg 9240
gtgcggtggt agccgtagat gtacctggac atccaggtga tgccggcggc ggtggtggag 9300
gcgcgcggga actcgcggac gcggttccag atgttgcgca gcggcaggaa gtagttcatg 9360
gtgggcacgg tctggcccgt gaggcgcgcg cagtcgtgga tgctctatac gggcaaaaac 9420
gaaagcggtc agcggctcga ctccgtggcc tggaggctaa gcgaacgggt tgggctgcgc 9480
gtgtaccccg gttcgaatct cgaatcaggc tggagccgca gctaacgtgg tattggcact 9540
cccgtctcga cccaagcctg caccaaccct ccaggatacg gaggcgggtc gttttgcaac 9600
ttttttttgg aggccggatg agactagtaa gcgcggaaag cggccgaccg cgatggctcg 9660
ctgccgtagt ctggagaaga atcgccaggg ttgcgttgcg gtgtgccccg gttcgaggcc 9720
ggccggattc cgcggctaac gagggcgtgg ctgccccgtc gtttccaaga ccccatagcc 9780
agccgacttc tccagttacg gagcgagccc ctcttttgtt ttgtttgttt ttgccagatg 9840
catcccgtac tgcggcagat gcgcccccac caccctccac cgcaacaaca gccccctcca 9900
cagccggcgc ttctgccccc gccccagcag caacttccag ccacgaccgc cgcggccgcc 9960
gtgagcgggg ctggacagag ttatgatcac cagctggcct tggaagaggg cgaggggctg 10020
gcgcgcctgg gggcgtcgtc gccggagcgg cacccgcgcg tgcagatgaa aagggacgct 10080
cgcgaggcct acgtgcccaa gcagaacctg ttcagagaca ggagcggcga ggagcccgag 10140
gagatgcgcg cggcccggtt ccacgcgggg cgggagctgc ggcgcggcct ggaccgaaag 10200
agggtgctga gggacgagga tttcgaggcg gacgagctga cggggatcag ccccgcgcgc 10260
gcgcacgtgg ccgcggccaa cctggtcacg gcgtacgagc agaccgtgaa ggaggagagc 10320
aacttccaaa aatccttcaa caaccacgtg cgcaccctga tcgcgcgcga ggaggtgacc 10380
ctgggcctga tgcacctgtg ggacctgctg gaggccatcg tgcagaaccc caccagcaag 10440
ccgctgacgg cgcagctgtt cctggtggtg cagcatagtc gggacaacga agcgttcagg 10500
gaggcgctgc tgaatatcac cgagcccgag ggccgctggc tcctggacct ggtgaacatt 10560
ctgcagagca tcgtggtgca ggagcgcggg ctgccgctgt ccgagaagct ggcggccatc 10620
aacttctcgg tgctgagttt gggcaagtac tacgctagga agatctacaa gaccccgtac 10680
gtgcccatag acaaggaggt gaagatcgac gggttttaca tgcgcatgac cctgaaagtg 10740
ctgaccctga gcgacgatct gggggtgtac cgcaacgaca ggatgcaccg tgcggtgagc 10800
gccagcaggc ggcgcgagct gagcgaccag gagctgatgc atagtctgca gcgggccctg 10860
accggggccg ggaccgaggg ggagagctac tttgacatgg gcgcggacct gcactggcag 10920
cccagccgcc gggccttgga ggcggcggca ggaccctacg tagaagaggt ggacgatgag 10980
gtggacgagg agggcgagta cctggaagac tgatggcgcg accgtatttt tgctagatgc 11040
aacaacaaca gccacctcct gatcccgcga tgcgggcggc gctgcagagc cagccgtccg 11100
gcattaactc ctcggacgat tggacccagg ccatgcaacg catcatggcg ctgacgaccc 11160
gcaaccccga agcctttaga cagcagcccc aggccaaccg gctctcggcc atcctggagg 11220
ccgtggtgcc ctcgcgctcc aaccccacgc acgagaaggt cctggccatc gtgaacgcgc 11280
tggtggagaa caaggccatc cgcggcgacg aggccggcct ggtgtacaac gcgctgctgg 11340
agcgcgtggc ccgctacaac agcaccaacg tgcagaccaa cctggaccgc atggtgaccg 11400
acgtgcgcga ggccgtggcc cagcgcgagc ggttccaccg cgagtccaac ctgggatcca 11460
tggtggcgct gaacgccttc ctcagcaccc agcccgccaa cgtgccccgg ggccaggagg 11520
actacaccaa cttcatcagc gccctgcgcc tgatggtgac cgaggtgccc cagagcgagg 11580
tgtaccagtc cgggccggac tacttcttcc agaccagtcg ccagggcttg cagaccgtga 11640
acctgagcca ggctttcaag aacttgcagg gcctgtgggg cgtgcaggcc ccggtcgggg 11700
accgcgcgac ggtgtcgagc ctgctgacgc cgaactcgcg cctgctgctg ctgctggtgg 11760
cccccttcac ggacagcggc agcatcaacc gcaactcgta cctgggctac ctgattaacc 11820
tgtaccgcga ggccatcggc caggcgcacg tggacgagca gacctaccag gagatcaccc 11880
acgtgagccg cgccctgggc caggacgacc cgggcaacct ggaagccacc ctgaactttt 11940
tgctgaccaa ccggtcgcag aagatcccgc cccagtacgc gctcagcacc gaggaggagc 12000
gcatcctgcg ttacgtgcag cagagcgtgg gcctgttcct gatgcaggag ggggccaccc 12060
ccagcgccgc gctcgacatg accgcgcgca acatggagcc cagcatgtac gccagcaacc 12120
gcccgttcat caataaactg atggactact tgcatcgggc ggccgccatg aactctgact 12180
atttcaccaa cgccatcctg aatccccact ggctcccgcc gccggggttc tacacgggcg 12240
agtacgacat gcccgacccc aatgacgggt tcctgtggga cgatgtggac agcagcgtgt 12300
tctccccccg accgggtgct aacgagcgcc ccttgtggaa gaaggaaggc agcgaccgac 12360
gcccgtcctc ggcgctgtcc ggccgcgagg gtgctgccgc ggcggtgccc gaggccgcca 12420
gtcctttccc gagcttgccc ttctcgctga acagtatccg cagcagcgag ctgggcagga 12480
tcacgcgccc gcgcttgctg ggcgaagagg agtacttgaa tgactcgctg ttgagacccg 12540
agcgggagaa gaacttcccc aataacggga tagaaagcct ggtggacaag atgagccgct 12600
ggaagacgta tgcgcaggag cacagggacg atccccgggc gtcgcagggg gccacgagcc 12660
ggggcagcgc cgcccgtaaa cgccggtggc acgacaggca gcggggacag atgtgggacg 12720
atgaggactc cgccgacgac agcagcgtgt tggacttggg tgggagtggt aacccgttcg 12780
ctcacctgcg cccccgtatc gggcgcatga tgtaagagaa accgaaaata aatgatactc 12840
accaaggcca tggcgaccag cgtgcgttcg tttcttctct gttgttgttg tatctagtat 12900
gatgaggcgt gcgtacccgg agggtcctcc tccctcgtac gagagcgtga tgcagcaggc 12960
gatggcggcg gcggcgatgc agcccccgct ggaggctcct tacgtgcccc cgcggtacct 13020
ggcgcctacg gaggggcgga acagcattcg ttactcggag ctggcaccct tgtacgatac 13080
cacccggttg tacctggtgg acaacaagtc ggcggacatc gcctcgctga actaccagaa 13140
cgaccacagc aacttcctga ccaccgtggt gcagaacaat gacttcaccc ccacggaggc 13200
cagcacccag accatcaact ttgacgagcg ctcgcggtgg ggcggccagc tgaaaaccat 13260
catgcacacc aacatgccca acgtgaacga gttcatgtac agcaacaagt tcaaggcgcg 13320
ggtgatggtc tcccgcaaga cccccaatgg ggtgacagtg acagaggatt atgatggtag 13380
tcaggatgag ctgaagtatg aatgggtgga atttgagctg cccgaaggca acttctcggt 13440
gaccatgacc atcgacctga tgaacaacgc catcatcgac aattacttgg cggtggggcg 13500
gcagaacggg gtgctggaga gcgacatcgg cgtgaagttc gacactagga acttcaggct 13560
gggctgggac cccgtgaccg agctggtcat gcccggggtg tacaccaacg aggctttcca 13620
tcccgatatt gtcttgctgc ccggctgcgg ggtggacttc accgagagcc gcctcagcaa 13680
cctgctgggc attcgcaaga ggcagccctt ccaggaaggc ttccagatca tgtacgagga 13740
tctggagggg ggcaacatcc ccgcgctcct ggatgtcgac gcctatgaga aaagcaagga 13800
ggatgcagca gctgaagcaa ctgcagccgt agctaccgcc tctaccgagg tcaggggcga 13860
taattttgca agcgccgcag cagtggcagc ggccgaggcg gctgaaaccg aaagtaagat 13920
agtcattcag ccggtggaga aggatagcaa gaacaggagc tacaacgtac taccggacaa 13980
gataaacacc gcctaccgca gctggtacct agcctacaac tatggcgacc ccgagaaggg 14040
cgtgcgctcc tggacgctgc tcaccacctc ggacgtcacc tgcggcgtgg agcaagtcta 14100
ctggtcgctg cccgacatga tgcaagaccc ggtcaccttc cgctccacgc gtcaagttag 14160
caactacccg gtggtgggcg ccgagctcct gcccgtctac tccaagagct tcttcaacga 14220
gcaggccgtc tactcgcagc agctgcgcgc cttcacctcg cttacgcacg tcttcaaccg 14280
cttccccgag aaccagatcc tcgtccgccc gcccgcgccc accattacca ccgtcagtga 14340
aaacgttcct gctctcacag atcacgggac cctgccgctg cgcagcagta tccggggagt 14400
ccagcgcgtg accgttactg acgccagacg ccgcacctgc ccctacgtct acaaggccct 14460
gggcatagtc gcgccgcgcg tcctctcgag ccgcaccttc taaatgtcca ttctcatctc 14520
gcccagtaat aacaccggtt ggggcctgcg cgcgcccagc aagatgtacg gaggcgctcg 14580
ccaacgctcc acgcaacacc ccgtgcgcgt gcgcgggcac ttccgcgctc cctggggcgc 14640
cctcaagggc cgcgtgcggt cgcgcaccac cgtcgacgac gtgatcgacc aggtggtggc 14700
cgacgcgcgc aactacaccc ccgccgccgc gcccgtctcc accgtggacg ccgtcatcga 14760
cagcgtggtg gcggacgcgc gccggtacgc ccgcgccaag agccggcggc ggcgcatcgc 14820
ccggcggcac cggagcaccc ccgccatgcg cgcggcgcga gccttgctgc gcagggccag 14880
gcgcacggga cgcagggcca tgctcagggc ggccagacgc gcggcttcag gcgccagcgc 14940
cggcaggacc cggagacgcg cggccacggc ggcggcagcg gccatcgcca gcatgtcccg 15000
cccgcggcga gggaacgtgt actgggtgcg cgacgccgcc accggtgtgc gcgtgcccgt 15060
gcgcacccgc ccccctcgca cttgaagatg ttcacttcgc gatgttgatg tgtcccagcg 15120
gcgaggagga tgtccaagcg caaattcaag gaagagatgc tccaggtcat cgcgcctgag 15180
atctacggcc ctgcggtggt gaaggaggaa agaaagcccc gcaaaatcaa gcgggtcaaa 15240
aaggacaaaa aggaagaaga aagtgatgtg gacggattgg tggagtttgt gcgcgagttc 15300
gccccccggc ggcgcgtgca gtggcgcggg cggaaggtgc aaccggtgct gagacccggc 15360
accaccgtgg tcttcacgcc cggcgagcgc tccggcaccg cttccaagcg ctcctacgac 15420
gaggtgtacg gggatgatga tattctggag caggcggccg agcgcctggg cgagtttgct 15480
tacggcaagc gcagccgttc cgcaccgaag gaagaggcgg tgtccatccc gctggaccac 15540
ggcaacccca cgccgagcct caagcccgtg accttgcagc aggtgctgcc gaccgcggcg 15600
ccgcgccggg ggttcaagcg cgagggcgag gatctgtacc ccaccatgca gctgatggtg 15660
cccaagcgcc agaagctgga agacgtgctg gagaccatga aggtggaccc ggacgtgcag 15720
cccgaggtca aggtgcggcc catcaagcag gtggccccgg gcctgggcgt gcagaccgtg 15780
gacatcaaga ttcccacgga gcccatggaa acgcagaccg agcccatgat caagcccagc 15840
accagcacca tggaggtgca gacggatccc tggatgccat cggctcctag tcgaagaccc 15900
cggcgcaagt acggcgcggc cagcctgctg atgcccaact acgcgctgca tccttccatc 15960
atccccacgc cgggctaccg cggcacgcgc ttctaccgcg gtcataccag cagccgccgc 16020
cgcaagacca ccactcgccg ccgccgtcgc cgcaccgccg ctgcaaccac ccctgccgcc 16080
ctggtgcgga gagtgtaccg ccgcggccgc gcacctctga ccctgccgcg cgcgcgctac 16140
cacccgagca tcgccattta aactttcgcc agctttgcag atcaatggcc ctcacatgcc 16200
gccttcgcgt tcccattacg ggctaccgag gaagaaaacc gcgccgtaga aggctggcgg 16260
ggaacgggat gcgtcgccac caccaccggc ggcggcgcgc catcagcaag cggttggggg 16320
gaggcttcct gcccgcgctg atccccatca tcgccgcggc gatcggggcg atccccggca 16380
ttgcttccgt ggcggtgcag gcctctcagc gccactgaga cacacttgga aacatcttgt 16440
aataaaccca tggactctga cgctcctggt cctgtgatgt gttttcgtag acagatggaa 16500
gacatcaatt tttcgtccct ggctccgcga cacggcacgc ggccgttcat gggcacctgg 16560
agcgacatcg gcaccagcca actgaacggg ggcgccttca attggagcag tctctggagc 16620
gggcttaaga atttcgggtc cacgcttaaa acctatggca gcaaggcgtg gaacagcacc 16680
acagggcagg cgctgaggga taagctgaaa gagcagaact tccagcagaa ggtggtcgat 16740
gggctcgcct cgggcatcaa cggggtggtg gacctggcca accaggccgt gcagcggcag 16800
atcaacagcc gcctggaccc ggtgccgccc gccggctccg tggagatgcc gcaggtggag 16860
gaggagctgc ctcccctgga caagcggggc gagaagcgac cccgccccga tgcggaggag 16920
acgctgctga cgcacacgga cgagccgccc ccgtacgagg aggcggtgaa actgggtctg 16980
cccaccacgc ggcccatcgc gcccctggcc accggggtgc tgaaacccga aaagcccgcg 17040
accctggact tgcctcctcc ccagccttcc cgcccctcta cagtggctaa gcccctgccg 17100
ccggtggccg tggcccgcgc gcgacccggg ggcaccgccc gccctcatgc gaactggcag 17160
agcactctga acagcatcgt gggtctggga gtgcagagtg tgaagcgccg ccgctgctat 17220
taaacctacc gtagcgctta acttgcttgt ctgtgtgtgt atgtattatg tcgccgccgc 17280
cgctgtccac cagaaggagg agtgaagagg cgcgtcgccg agttgcaaga tggccacccc 17340
atcgatgctg ccccagtggg cgtacatgca catcgccgga caggacgctt cggagtacct 17400
gagtccgggt ctggtgcagt ttgcccgcgc cacagacacc tacttcagtc tggggaacaa 17460
gtttaggaac cccacggtgg cgcccacgca cgatgtgacc accgaccgca gccagcggct 17520
gacgctgcgc ttcgtgcccg tggaccgcga ggacaacacc tactcgtaca aagtgcgcta 17580
cacgctggcc gtgggcgaca accgcgtgct ggacatggcc agcacctact ttgacatccg 17640
cggcgtgctg gatcggggcc ctagcttcaa accctactcc ggcaccgcct acaacagtct 17700
ggcccccaag ggagcaccca acacttgtca gtggacatat aaagccgatg gtgaaactgc 17760
cacagaaaaa acctatacat atggaaatgc acccgtgcag ggcattaaca tcacaaaaga 17820
tggtattcaa cttggaactg acaccgatga tcagccaatc tacgcagata aaacctatca 17880
gcctgaacct caagtgggtg atgctgaatg gcatgacatc actggtactg atgaaaagta 17940
tggaggcaga gctcttaagc ctgataccaa aatgaagcct tgttatggtt cttttgccaa 18000
gcctactaat aaagaaggag gtcaggcaaa tgtgaaaaca ggaacaggca ctactaaaga 18060
atatgacata gacatggctt tctttgacaa cagaagtgcg gctgctgctg gcctagctcc 18120
agaaattgtt ttgtatactg aaaatgtgga tttggaaact ccagataccc atattgtata 18180
caaagcaggc acagatgaca gcagctcttc tattaatttg ggtcagcaag ccatgcccaa 18240
cagacctaac tacattggtt tcagagacaa ctttatcggg ctcatgtact acaacagcac 18300
tggcaatatg ggggtgctgg ccggtcaggc ttctcagctg aatgctgtgg ttgacttgca 18360
agacagaaac accgagctgt cctaccagct cttgcttgac tctctgggtg acagaacccg 18420
gtatttcagt atgtggaatc aggcggtgga cagctatgat cctgatgtgc gcattattga 18480
aaatcatggt gtggaggatg aacttcccaa ctattgtttc cctctggatg ctgttggcag 18540
aacagatact tatcagggaa ttaaggctaa tggaactgat caaaccacat ggaccaaaga 18600
tgacagtgtc aatgatgcta atgagatagg caagggtaat ccattcgcca tggaaatcaa 18660
catccaagcc aacctgtgga ggaacttcct ctacgccaac gtggccctgt acctgcccga 18720
ctcttacaag tacacgccgg ccaatgttac cctgcccacc aacaccaaca cctacgatta 18780
catgaacggc cgggtggtgg cgccctcgct ggtggactcc tacatcaaca tcggggcgcg 18840
ctggtcgctg gatcccatgg acaacgtgaa ccccttcaac caccaccgca atgcggggct 18900
gcgctaccgc tccatgctcc tgggcaacgg gcgctacgtg cccttccaca tccaggtgcc 18960
ccagaaattt ttcgccatca agagcctcct gctcctgccc gggtcctaca cctacgagtg 19020
gaacttccgc aaggacgtca acatgatcct gcagagctcc ctcggcaacg acctgcgcac 19080
ggacggggcc tccatctcct tcaccagcat caacctctac gccaccttct tccccatggc 19140
gcacaacacg gcctccacgc tcgaggccat gctgcgcaac gacaccaacg accagtcctt 19200
caacgactac ctctcggcgg ccaacatgct ctaccccatc ccggccaacg ccaccaacgt 19260
gcccatctcc atcccctcgc gcaactgggc cgccttccgc ggctggtcct tcacgcgtct 19320
caagaccaag gagacgccct cgctgggctc cgggttcgac ccctacttcg tctactcggg 19380
ctccatcccc tacctcgacg gcaccttcta cctcaaccac accttcaaga aggtctccat 19440
caccttcgac tcctccgtca gctggcccgg caacgaccgg ctcctgacgc ccaacgagtt 19500
cgaaatcaag cgcaccgtcg acggcgaggg ctacaacgtg gcccagtgca acatgaccaa 19560
ggactggttc ctggtccaga tgctggccca ctacaacatc ggctaccagg gcttctacgt 19620
gcccgagggc tacaaggacc gcatgtactc cttcttccgc aacttccagc ccatgagccg 19680
ccaggtggtg gacgaggtca actacaagga ctaccaggcc gtcaccctgg cctaccagca 19740
caacaactcg ggcttcgtcg gctacctcgc gcccaccatg cgccagggcc agccctaccc 19800
cgccaactac ccctacccgc tcatcggcaa gagcgccgtc accagcgtca cccagaaaaa 19860
gttcctctgc gacagggtca tgtggcgcat ccccttctcc agcaacttca tgtccatggg 19920
cgcgctcacc gacctcggcc agaacatgct ctatgccaac tccgcccacg cgctagacat 19980
gaatttcgaa gtcgacccca tggatgagtc cacccttctc tatgttgtct tcgaagtctt 20040
cgacgtcgtc cgagtgcacc agccccaccg cggcgtcatc gaggccgtct acctgcgcac 20100
ccccttctcg gccggtaacg ccaccaccta agctcttgct tcttgcaagc catggccgcg 20160
ggctccggcg agcaggagct cagggccatc atccgcgacc tgggctgcgg gccctacttc 20220
ctgggcacct tcgataagcg cttcccggga ttcatggccc cgcacaagct ggcctgcgcc 20280
atcgtcaaca cggccggccg cgagaccggg ggcgagcact ggctggcctt cgcctggaac 20340
ccgcgctcga acacctgcta cctcttcgac cccttcgggt tctcggacga gcgcctcaag 20400
cagatctacc agttcgagta cgagggcctg ctgcgccgca gcgccctggc caccgaggac 20460
cgctgcgtca ccctggaaaa gtccacccag accgtgcagg gtccgcgctc ggccgcctgc 20520
gggctcttct gctgcatgtt cctgcacgcc ttcgtgcact ggcccgaccg ccccatggac 20580
aagaacccca ccatgaactt gctgacgggg gtgcccaacg gcatgctcca gtcgccccag 20640
gtggaaccca ccctgcgccg caaccaggag gcgctctacc gcttcctcaa ctcccactcc 20700
gcctactttc gctcccaccg cgcgcgcatc gagaaggcca ccgccttcga ccgcatgaat 20760
caagacatgt aaaccgtgtg tgtatgttaa atgtctttaa taaacagcac tttcatgtta 20820
cacatgcatc tgagatgatt tatttagaaa tcgaaagggt tctgccgggt ctcggcatgg 20880
cccgcgggca gggacacgtt gcggaactgg tacttggcca gccacttgaa ctcggggatc 20940
agcagtttgg gcagcggggt gtcggggaag gagtcggtcc acagcttccg cgtcagttgc 21000
agggcgccca gcaggtcggg cgcggagatc ttgaaatcgc agttgggacc cgcgttctgc 21060
gcgcgggagt tgcggtacac ggggttgcag cactggaaca ccatcagggc cgggtgcttc 21120
acgctcgcca gcaccgtcgc gtcggtgatg ctctccacgt cgaggtcctc ggcgttggcc 21180
atcccgaagg gggtcatctt gcaggtctgc cttcccatgg tgggcacgca cccgggcttg 21240
tggttgcaat cgcagtgcag ggggatcagc atcatctggg cctggtcggc gttcatcccc 21300
gggtacatgg ccttcatgaa agcctccaat tgcctgaacg cctgctgggc cttggctccc 21360
tcggtgaaga agaccccgca ggacttgcta gagaactggt tggtggcgca cccggcgtcg 21420
tgcacgcagc agcgcgcgtc gttgttggcc agctgcacca cgctgcgccc ccagcggttc 21480
tgggtgatct tggcccggtc ggggttctcc ttcagcgcgc gctgcccgtt ctcgctcgcc 21540
acatccatct cgatcatgtg ctccttctgg atcatggtgg tcccgtgcag gcaccgcagc 21600
ttgccctcgg cctcggtgca cccgtgcagc cacagcgcgc acccggtgca ctcccagttc 21660
ttgtgggcga tctgggaatg cgcgtgcacg aagccctgca ggaagcggcc catcatggtg 21720
gtcagggtct tgttgctagt gaaggtcagc ggaatgccgc ggtgctcctc gttgatgtac 21780
aggtggcaga tgcggcggta cacctcgccc tgctcgggca tcagctggaa gttggctttc 21840
aggtcggtct ccacgcggta gcggtccatc agcatagtca tgatttccat acccttctcc 21900
caggccgaga cgatgggcag gctcataggg ttcttcacca tcatcttagc gctagcagcc 21960
gcggccaggg ggtcgctctc gtccagggtc tcaaagctcc gcttgccgtc cttctcggtg 22020
atccgcaccg gggggtagct gaagcccacg gccgccagct cctcctcggc ctgtctttcg 22080
tcctcgctgt cctggctgac gtcctgcagg accacatgct tggtcttgcg gggtttcttc 22140
ttgggcggca gcggcggcgg agatgttgga gatggcgagg gggagcgcga gttctcgctc 22200
accactacta tctcttcctc ttcttggtcc gaggccacgc ggcggtaggt atgtctcttc 22260
gggggcagag gcggaggcga cgggctctcg ccgccgcgac ttggcggatg gctggcagag 22320
ccccttccgc gttcgggggt gcgctcccgg cggcgctctg actgacttcc tccgcggccg 22380
gccattgtgt tctcctaggg aggaacaaca agcatggaga ctcagccatc gccaacctcg 22440
ccatctgccc ccaccgccga cgagaagcag cagcagcaga atgaaagctt aaccgccccg 22500
ccgcccagcc ccgccacctc cgacgcggcc gtcccagaca tgcaagagat ggaggaatcc 22560
atcgagattg acctgggcta tgtgacgccc gcggagcacg aggaggagct ggcagtgcgc 22620
ttttcacaag aagagataca ccaagaacag ccagagcagg aagcagagaa tgagcagagt 22680
caggctgggc tcgagcatga cggcgactac ctccacctga gcggggggga ggacgcgctc 22740
atcaagcatc tggcccggca ggccaccatc gtcaaggatg cgctgctcga ccgcaccgag 22800
gtgcccctca gcgtggagga gctcagccgc gcctacgagt tgaacctctt ctcgccgcgc 22860
gtgcccccca agcgccagcc caatggcacc tgcgagccca acccgcgcct caacttctac 22920
ccggtcttcg cggtgcccga ggccctggcc acctaccaca tctttttcaa gaaccaaaag 22980
atccccgtct cctgccgcgc caaccgcacc cgcgccgacg cccttttcaa cctgggtccc 23040
ggcgcccgcc tacctgatat cgcctccttg gaagaggttc ccaagatctt cgagggtctg 23100
ggcagcgacg agactcgggc cgcgaacgct ctgcaaggag aaggaggaga gcatgagcac 23160
cacagcgccc tggtcgagtt ggaaggcgac aacgcgcggc tggcggtgct caaacgcacg 23220
gtcgagctga cccatttcgc ctacccggct ctgaacctgc cccccaaagt catgagcgcg 23280
gtcatggacc aggtgctcat caagcgcgcg tcgcccatct ccgaggacga gggcatgcaa 23340
gactccgagg agggcaagcc cgtggtcagc gacgagcagc tggcccggtg gctgggtcct 23400
aatgctagtc cccagagttt ggaagagcgg cgcaaactca tgatggccgt ggtcctggtg 23460
accgtggagc tggagtgcct gcgccgcttc ttcgccgacg cggagaccct gcgcaaggtc 23520
gaggagaacc tgcactacct cttcaggcac gggttcgtgc gccaggcctg caagatctcc 23580
aacgtggagc tgaccaacct ggtctcctac atgggcatct tgcacgagaa ccgcctgggg 23640
cagaacgtgc tgcacaccac cctgcgcggg gaggcccggc gcgactacat ccgcgactgc 23700
gtctacctct acctctgcca cacctggcag acgggcatgg gcgtgtggca gcagtgtctg 23760
gaggagcaga acctgaaaga gctctgcaag ctcctgcaga agaacctcaa gggtctgtgg 23820
accgggttcg acgagcgcac caccgcctcg gacctggccg acctcatttt ccccgagcgc 23880
ctcaggctga cgctgcgcaa cggcctgccc gactttatga gccaaagcat gttgcaaaac 23940
tttcgctctt tcatcctcga acgctccgga atcctgcccg ccacctgctc cgcgctgccc 24000
tcggacttcg tgccgctgac cttccgcgag tgccccccgc cgctgtggag ccactgctac 24060
ctgctgcgcc tggccaacta cctggcctac cactcggacg tgatcgagga cgtcagcggc 24120
gagggcctgc tcgagtgcca ctgccgctgc aacctctgca cgccgcaccg ctccctggcc 24180
tgcaaccccc agctgctgag cgagacccag atcatcggca ccttcgagtt gcaagggccc 24240
agcgaaggcg agggttcagc cgccaagggg ggtctgaaac tcaccccggg gctgtggacc 24300
tcggcctact tgcgcaagtt cgtgcccgag gactaccatc ccttcgagat caggttctac 24360
gaggaccaat cccatccgcc caaggccgag ctgtcggcct gcgtcatcac ccagggggcg 24420
atcctggccc aattgcaagc catccagaaa tcccgccaag aattcttgct gaaaaagggc 24480
cgcggggtct acctcgaccc ccagaccggt gaggagctca accccggctt cccccaggat 24540
gccccgagga aacaagaagc tgaaagtgga gctgccgccc gtggaggatt tggaggaaga 24600
ctgggagaac agcagtcagg cagaggagga ggagatggag gaagactggg acagcactca 24660
ggcagaggag gacagcctgc aagacagtct ggaggaagac gaggaggagg cagaggagga 24720
ggtggaagaa gcagccgccg ccagaccgtc gtcctcggcg ggggagaaag caagcagcac 24780
ggataccatc tccgctccgg gtcggggtcc cgctcgacca cacagtagat gggacgagac 24840
cggacgattc ccgaacccca ccacccagac cggtaagaag gagcggcagg gatacaagtc 24900
ctggcggggg cacaaaaacg ccatcgtctc ctgcttgcag gcctgcgggg gcaacatctc 24960
cttcacccgg cgctacctgc tcttccaccg cggggtgaac tttccccgca acatcttgca 25020
ttactaccgt cacctccaca gcccctacta cttccaagaa gaggcagcag cagcagaaaa 25080
agaccagcag aaaaccagca gctagaaaat ccacagcggc ggcagcaggt ggactgagga 25140
tcgcggcgaa cgagccggcg caaacccggg agctgaggaa ccggatcttt cccaccctct 25200
atgccatctt ccagcagagt cgggggcagg agcaggaact gaaagtcaag aaccgttctc 25260
tgcgctcgct cacccgcagt tgtctgtatc acaagagcga agaccaactt cagcgcactc 25320
tcgaggacgc cgaggctctc ttcaacaagt actgcgcgct cactcttaaa gagtagcccg 25380
cgcccgccca gtcgcagaaa aaggcgggaa ttacgtcacc tgtgcccttc gccctagccg 25440
cctccaccca tcatcatgag caaagagatt cccacgcctt acatgtggag ctaccagccc 25500
cagatgggcc tggccgccgg tgccgcccag gactactcca cccgcatgaa ttggctcagc 25560
gccgggcccg cgatgatctc acgggtgaat gacatccgcg cccaccgaaa ccagatactc 25620
ctagaacagt cagcgctcac cgccacgccc cgcaatcacc tcaatccgcg taattggccc 25680
gccgccctgg tgtaccagga aattccccag cccacgaccg tactacttcc gcgagacgcc 25740
caggccgaag tccagctgac taactcaggt gtccagctgg cgggcggcgc caccctgtgt 25800
cgtcaccgcc ccgctcaggg tataaagcgg ctggtgatcc ggggcagagg cacacagctc 25860
aacgacgagg tggtgagctc ttcgctgggt ctgcgacctg acggagtctt ccaactcgcc 25920
ggatcgggga gatcttcctt cacgcctcgt caggccgtcc tgactttgga gagttcgtcc 25980
tcgcagcccc gctcgggtgg catcggcact ctccagttcg tggaggagtt cactccctcg 26040
gtctacttca accccttctc cggctccccc ggccactacc cggacgagtt catcccgaac 26100
ttcgacgcca tcagcgagtc ggtggacggc tacgattgag tttaaactca cccccttatc 26160
cagtgaaata aagatcatat tgatgatgat tttacagaaa taaaaaataa tcatttgatt 26220
tgaaataaag atacaatcat attgatgatt tgagtttaac aaaaaaataa agaatcactt 26280
acttgaaatc tgataccagg tctctgtcca tgttttctgc caacaccact tcactcccct 26340
cttcccagct ctggtactgc aggccccggc gggctgcaaa cttcctccac acgctgaagg 26400
ggatgtcaaa ttcctcctgt ccctcaatct tcattttatc ttctatcaga tgtccaaaaa 26460
gcgcgtccgg gtggatgatg acttcgaccc cgtctacccc tacgatgcag acaacgcacc 26520
gaccgtgccc ttcatcaacc cccccttcgt ctcttcagat ggattccaag agaagcccct 26580
gggggtgttg tccctgcgac tggccgaccc cgtcaccacc aagaacgggg aaatcaccct 26640
caagctggga gagggggtgg acctcgattc ctcgggaaaa ctcatctcca acacggccac 26700
caaggccgcc gcccctctca gtttttccaa caacaccatt tcccttaaca tggatcaccc 26760
cttttacact aaagatggaa aattatcctt acaagtttct ccaccattaa atatactgag 26820
aacaagcatt ctaaacacac tagctttagg ttttggatca ggtttaggac tccgtggctc 26880
tgccttggca gtacagttag tctctccact tacatttgat actgatggaa acataaagct 26940
taccttagac agaggtttgc atgttacaac aggagatgca attgaaagca acataagctg 27000
ggctaaaggt ttaaaatttg aagatggagc catagcaacc aacattggaa atgggttaga 27060
gtttggaagc agtagtacag aaacaggtgt tgatgatgct tacccaatcc aagttaaact 27120
tggatctggc cttagctttg acagtacagg agccataatg gctggtaaca aagaagacga 27180
taaactcact ttgtggacaa cacctgatcc atcaccaaac tgtcaaatac tcgcagaaaa 27240
tgatgcaaaa ctaacacttt gcttgactaa atgtggtagt caaatactgg ccactgtgtc 27300
agtcttagtt gtaggaagtg gaaacctaaa ccccattact ggcaccgtaa gcagtgctca 27360
ggtgtttcta cgttttgatg caaacggtgt tcttttaaca gaacattcta cactaaaaaa 27420
atactggggg tataggcagg gagatagcat agatggcact ccatatacca atgctgtagg 27480
attcatgccc aatttaaaag cttatccaaa gtcacaaagt tctactacta aaaataatat 27540
agtagggcaa gtatacatga atggagatgt ttcaaaacct atgcttctca ctataaccct 27600
caatggtact gatgacagca acagtacata ttcaatgtca ttttcataca cctggactaa 27660
tggaagctat gttggagcaa catttggggc taactcttat accttctcat acatcgccca 27720
agaatgaaca ctgtatccca ccctgcatgc caacccttcc caccccactc tgtggaacaa 27780
actctgaaac acaaaataaa ataaagttca agtgttttat tgattcaaca gtttcacaga 27840
accctagtat tcaacctgcc acctccctcc caacacacag agtacacagt cctttctccc 27900
cggctggcct taaaaagcat catatcatgg gtaacagaca tattcttagg tgttatattc 27960
cacacggttt cctgtcgagc caaacgctca tcagtgatat taataaactc cccgggcagc 28020
tcacttaagt tcatgtcgct gtccagctgc tgagccacag gctgctgtcc aacttgcggt 28080
tgcttaacgg gcggcgaagg agaagtccac gcctacatgg gggtagagtc ataatcgtgc 28140
atcaggatag ggcggtggtg ctgcagcagc gcgcgaataa actgctgccg ccgccgctcc 28200
gtcctgcagg aatacaacat ggcagtggtc tcctcagcga tgattcgcac cgcccgcagc 28260
ataaggcgcc ttgtcctccg ggcacagcag cgcaccctga tctcacttaa atcagcacag 28320
taactgcagc acagcaccac aatattgttc aaaatcccac agtgcaaggc gctgtatcca 28380
aagctcatgg cggggaccac agaacccacg tggccatcat accacaagcg caggtagatt 28440
aagtggcgac ccctcataaa cacgctggac ataaacatta cctcttttgg catgttgtaa 28500
ttcaccacct cccggtacca tataaacctc tgattaaaca tggcgccatc caccaccatc 28560
ctaaaccagc tggccaaaac ctgcccgccg gctatacact gcagggaacc gggactggaa 28620
caatgacagt ggagagccca ggactcgtaa ccatggatca tcatgctcgt catgatatca 28680
atgttggcac aacacaggca cacgtgcata cacttcctca ggattacaag ctcctcccgc 28740
gttagaacca tatcccaggg aacaacccat tcctgaatca gcgtaaatcc cacactgcag 28800
ggaagacctc gcacgtaact cacgttgtgc attgtcaaag tgttacattc gggcagcagc 28860
ggatgatcct ccagtatggt agcgcgggtt tctgtctcaa aaggaggtag acgatcccta 28920
ctgtacggag tgcgccgaga caaccgagat cgtgttggtc gtagtgtcat gccaaatgga 28980
acgccggacg tagtcatatt tcctgaagca aaaccaggtg cgggcgtgac aaacagatct 29040
gcgtctccgg tctcgccgct tagatcgctc tgtgtagtag ttgtagtata tccactctct 29100
caaagcatcc aggcgccccc tggcttcggg ttctatgtaa actccttcat gcgccgctgc 29160
cctgataaca tccaccaccg cagaataagc cacacccagc caacctacac attcgttctg 29220
cgagtcacac acgggaggag cgggaagagc tggaagaacc atgattaact ttattccaaa 29280
cggtctcgga gcacttcaaa atgcaggtcc cggaggtggc acctctcgcc cccactgtgt 29340
tggtggaaaa taacagccag gtcaaaggtg acacggttct cgagatgttc cacggtggct 29400
tccagcaaag cctccacgcg cacatccaga aacaagagga cagcgaaagc gggagcgttt 29460
tctaattcct caatcatcat attacactcc tgcaccatcc ccagataatt ttcatttttc 29520
cagccttgaa tgattcgtat tagttcctga ggtaaatcca agccagccat gataaaaagc 29580
tcgcgcagag cgccctccac cggcattctt aagcacaccc tcataattcc aagagattct 29640
gctcctggtt cacctgcagc agattaacaa tgggaatatc aaaatctctg ccgcgatccc 29700
taagctcctc cctcaacaat aactgtatgt aatctttcat atcatctccg aaatttttag 29760
ccatagggcc gccaggaata agagcagggc aagccacatt acagataaag cgaagtcctc 29820
cccagtgwgc attgccaaat gtaagattga aataagcatg ctggctagac cctgtgatat 29880
cttccagata actggacaga aaatcaggca agcaattttt aagaaaatca acaaaagaaa 29940
agtcgtccag gtgcaggttt agagcctcag gaacaacgat ggaataagtg caaggagtgc 30000
gttccagcat ggttagtgtt tttttggtga tctgtagaac aaaaaataaa catgcaatat 30060
taaaccatgc tagcctggcg aacaggtggg taaatcactc tttccagcac caggcaggct 30120
acggggtctc cggcgcgacc ctcgtagaag ctgtcgccat gattgaaaag catcaccgag 30180
agaccttccc ggtggccggc atggatgatt cgagaagaag catacactcc gggaacattg 30240
gcatccgtga gtgaaaaaaa gcgacctata aagcctcggg gcactacaat gctcaatctc 30300
aattccagca aagccacccc atgcggatgg agcacaaaat tggcaggtgc gtaaaaaatg 30360
taattactcc cctcctgcac aggcagcaaa gcccccgctc cctccagaaa cacatacaaa 30420
gcctcagcgt ccatagctta ccgagcacgg caggcgcaag agtcagagaa aaggctgagc 30480
tctaacctga ctgcccgctc ctgtgctcaa tatatagccc taacctacac tgacgtaaag 30540
gccaaagtct aaaaataccc gccaaataat cacacacgcc cagcacacgc ccagaaaccg 30600
gtgacacact caaaaaaata cgcgcacttc ctcaaacgcc caaaactgcc gtcatttccg 30660
ggttcccacg ctacgtcatc aaaacacgac tttcaaattc cgtcgaccgt taaaaacgtc 30720
acccgccccg cccctaacgg tcgcccgtct ctcagccaat cagcgccccg catccccaaa 30780
ttcaaacacc tcatttgcat attaacgcgc acaaaaagtt tgaggtatat tattgatgat 30840
gg 30842
SEQ ID NO: 6-bgh polyadenylation signal
ctgtgccttctagttgccagccatctgttgtttgcccctcccccgtgccttccttgaccctggaaggtgcc
actcccactgtcctttcctaataaaatgaggaaattgcatcgcattgtctgagtaggtgtcattctattct
ggggggtggggtggggcaggacagcaagggggaggattgggaagacaatagcaggcatgctggggatgcgg
tgggctctatgg
SEQ ID NO: 7: long CMV promoter
ATCGCCATTTTTCCAAAAGTGATTTTTGGGCATACGCGATATCTGGCGATAGCGCTTA
TATCGTTTACGGGGGATGGCGATAGACGATTTGGTGACTTGGGCGATTCTGTGTGT
CGCAAATATCGCATTTCGATATAGGTGACAGACGATATGAGGCTATATCGCCGATA
GAGGCGACATCAAGCTGGCACATGGCCAATGCATATCGATCTATACATTGAATCAATA
TTGGCCATTAGCCATATTATTCATTGGTTATATAGCATAAATCAATATTGGCTATTGG
CCATTGCATACGTTGTATCCATATCATAATATGTACATTTATATTGGCTCATGTCCAAC
ATTACCGCCATGTTGACATTGATTATTGACTAGTTATTAATAGTAATCAATTACGGGG
TCATTAGTTCATAGCCCATATATGGAGTTCCGCGTTACATAACTTACGGTAAATGGCC
CGCCTGGCTGACCGCCCAACGACCCCCGCCCATTGACGTCAATAATGACGTATGTTCC
CATAGTAACGCCAATAGGGACTTCCATTGACGTCAATGGGTGGAGTATTTACGGTAA
ACTGCCCACTTGGCAGTACATCAAGTGTATCATATGCCAAGTACGCCCCCTATTGACG
TCAATGACGGTAAATGGCCCGCCTGGCATTATGCCCAGTACATGACCTTATGGGACTT
TCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTC
CACCCCATTGACGTCAATGGGAGTTTGTTTGGCACCAAAATCAACGGGATTTCCAA
AATGTCGTAACAACTCCGCCCCATTGACGCAAATGGGCGGTAGGCGTGTACGGTGGG
AGGTCTATATAAGCAGAGCTCTCCCTATCAGTGATAGAGATCTCCCTATCAGTGATAG
AGATCGTCGACGAGCTCGTTTAGTGAACCGTCAGATCGCCTGGAGACGCCATCCACG
CTGTTTGACCTCCATAGAAGACACCGGGACCGATCCAGCCTCCGCGGCCGGGAACG
GTGCATTGGAACGCGGATTCCCCGTGCCAAGAGTGACGTAAGTACCGCCTATAGAGT
CTATAGGCCCACCCCCTTGGCTTCTTATGCATGCTATACTGTTTTTGGCTTGGGGTCT
ATACACCCCCGCTTCCTCATGTTATAGGTGATGGTATAGCTTAGCCTATAGGTGTGGG
TTATTGACCATTATTGACCACTCCCCTATTGGTGACGATACTTTCCATTACTAATCCAT
AACATGGCTTTGCCACAACTCTTTATTGGCTATATGCCAATACACTGTCCTTCAG
AGACTGACACGGACTCTGTATTTTTACAGGATGGGGTCTCATTATTATTTACAAATT
CACATATACAACACCACCGTCCCCAGTGCCCGCAGTTTTTATTAAACATAACGTGGGA
TCTCCACGCGAATCTCGGGTACGTGTTCCGGACATGGGCTCTTCTCCGGTAGCGGCG
GAGCTTCTACATCCGAGCCCTGCTCCCATGCCTCCAGCGACTCATGGTCGCTCGGCAG
CTCCTTGCTCCTAACAGTGGAGGCCAGACTTAGGCACAGCACGATGCCCACCACCACC
AGTGTGCCGCACAAGGCCGTGGCGGTAGGGTATGTGTCTGAAAATGAGCTCGGGGA
GCGGGCTTGCACCGCTGACGCATTTGGAAGACTTAAGGCAGCGGCAGAAGAAGATGC
AGGCAGCTGAGTTGTTGTGTTCTGATAAGAGTCAGAGGTAACTCCCGTTGCGGTGCT
GTTAACGGTGGAGGGCAGTGTAGTCTGAGCAGTACTCGTTGCTGCCGCGCGCGCCAC
CAGACATAATAGCTGACAGACTAACAGACTGTTCCTTTCCATGGGTCTTTTCTGCA

1. A composition comprising a viral vector comprising nucleic acid having a polynucleotide sequence encoding at least one epitope of the varicella-zoster virus (VZV) Gly E antigen, wherein the viral vector is an adenoviral vector.

2. The composition of claim 1 wherein the at least one epitope comprises at least one CD4 T cell epitope and at least one CD8 T cell epitope.

3. The composition of claim 1 wherein the adenoviral vector is of human or simian origin.

4. The composition of claim 1 wherein the adenoviral vector is selected from the group consisting of ChAdOx 1 and ChAdOx 2.

5. The composition of claim 1 wherein the composition is adjuvant-free.

6. The composition of claim 1 wherein the Gly E antigen has the amino acid sequence is selected from the group consisting of SEQ ID NO: 1 and SEQ ID NO: 2.

7. The composition of claim 1 wherein the polynucleotide sequence comprises a sequence selected from the group consisting of SEQ ID NO: 3 and SEQ ID NO: 4.

8. The composition of claim 1 wherein the polynucleotide sequence further comprises the sequence of the bgh polyadenylation signal SEQ ID NO: 6.

9. The composition of claim 1 wherein the polynucleotide sequence encoding at least one epitope of the varicella-zoster virus (VZV) Gly E antigen is operably connected to the long CMV promoter, wherein the long CMV promoter has the nucleotide sequence of SEQ ID NO: 7.

10. The composition of claim 1 wherein the viral vector sequence is as in ECACC accession number 12052403 (ChAdOx1).

11. The composition of claim 1 wherein the viral vector comprises the sequence of SEQ ID NO: 5 (ChAdOx2).

12. The composition of claim 1 wherein the composition is configured such that administration of a single dose of the composition to a mammalian subject induces protective immunity in the subject.

13. The composition of claim 1 wherein the composition is configured to induce an immune response against VZV in a subject by administration of the composition to the subject.

14. (canceled)

15. (canceled)

16. The composition of claim 1, wherein the composition is configured such that annual administration to a subject is capable of inducing an immune response against VZV in the subject.

17. The composition of claim 1, wherein the composition is configured for use in preventing VZV infection in a subject by administration of the composition to the subject.

18. The composition of claim 1, wherein the composition is configured for use in prevention of shingles in a subject by administration of the composition to the subject.

19. (canceled)

20. (canceled)

21. (canceled)

22. (canceled)

23. (canceled)

24. (canceled)

25. (canceled)

26. A method of inducing an immune response against varicella-zoster virus (VZV) in a mammalian subject, the method comprising the steps of administering at least one dose of the composition of claim 1 to the subject.

27. A method of preventing shingles in a mammalian subject, the method comprising the steps of administering at least one dose of the composition of claim 1 to the subject.

28. (canceled)

29. (canceled)

30. The method of claim 26 wherein the composition is administered annually.

31. The method of claim 26 wherein the composition is administered by a route of administration selected from the group consisting of subcutaneous, intradermal and intramuscular administration.

32. (canceled)

33. (canceled)

34. The composition of claim 1 wherein the composition is configured for use in treatment or prevention of chickenpox in a subject.


SEQUENCE LISTING (9 sequences)

SEQ ID NO: 1 - protein, 623 aa, Varicella zoster
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 2 - protein, 623 aa, Varicella zoster; identical to SEQ ID NO: 1

SEQ ID NO: 3 - DNA, 1872 bp, Varicella zoster
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtga

SEQ ID NO: 4 - DNA, 1872 bp, Varicella zoster
atgggcaccgtgaacaagcccgtcgtgggcgtgctgatgggcttcggcatcatcaccggcaccctgcggatcaccaatcctgtgcgggccagcgtgctgagatacgacgacttccacatcgacgaggacaagctggacaccaacagcgtgtacgagccctactaccacagcgaccacgccgagagcagctgggtcaacagaggcgagtccagccggaaggcctacgaccacaacagcccctacatctggccccggaacgactacgacggcttcctggaaaatgcccacgagcaccacggcgtgtacaaccagggcagaggcatcgacagcggcgagagactgatgcagcccacccagatgagcgcccaggaagatctgggcgacgacaccggcatccacgtgatccctaccctgaacggcgacgaccggcacaagatcgtgaacgtggaccagcggcagtacggcgacgtgttcaagggcgacctgaaccccaagccccagggacagcggctgattgaggtgtccgtggaagagaaccaccccttcaccctgagagcccccatccagagaatctacggcgtgcgctataccgagacttggagcttcctgcccagcctgacctgtactggcgacgccgctcctgccatccagcacatctgcctgaagcacaccacctgtttccaggacgtggtggtggacgtggactgcgccgagaacaccaaagaggaccagctggccgagatcagctaccggttccagggcaagaaagaggccgaccagccctggatcgtcgtgaacaccagcaccctgttcgacgagctggaactggacccccccgagattgaacccggggtgctgaaggtgctgcggaccgagaagcagtacctgggagtgtacatctggaacatgcggggcagcgacggcacctctacctacgccaccttcctcgtgacctggaagggcgacgagaaaacccggaaccctacccctgccgtgacccctcagcctagaggcgccgagtttcacatgtggaattaccacagccacgtgttcagcgtgggcgacaccttctccctggccatgcatctgcagtacaagatccacgaggcccccttcgacctgctgctggaatggctgtacgtgcccatcgaccctacctgccagcccatgcggctgtactccacctgtctgtaccaccccaacgccccccagtgcctgagccacatgaatagcggctgcaccttcaccagcccccacctggctcagagggtggccagcaccgtgtaccagaattgcgagcacgccgacaactacaccgcctactgcctgggcatcagccacatggaacctagcttcggcctgatcctgcacgacggcggcaccaccctgaagttcgtggataccccagagagcctgagcggcctgtacgtgttcgtggtgtacttcaacggccacgtggaagccgtggcctacaccgtggtgtccaccgtggaccacttcgtgaacgccatcgaggaacggggcttccctccaactgctggacagcctcctgccaccaccaagcccaaagaaatcacccccgtgaaccccggcaccagccctctgctgcgctatgctgcttggacaggcggactggctgctgtggtgctgctgtgcctcgtgattttcctgatctgcaccgccaagcggatgagagtgaaggcctatcgggtggacaagtccccctacaaccagagcatgtactacgccggcctgcccgtggacgatttcgaggatagcgagagcaccgacaccgaggaagagttcggcaacgccattggcggctctcacggcggcagcagctataccgtgtacatcgacaagacccgctga

SEQ ID NO: 5 - DNA, 30842 bp, Artificial Sequence
  ChAdOx2 Viral vector based on Chimpanzee adenovirus C68
ccatcttcaataatatacctcaaactttttgtgcgcgttaatatgcaaatgaggcgtttgaatttggggaggaagggcggtgattggtcgagggatgagcgaccgttaggggcggggcgagtgacgttttgatgacgtggttgcgaggaggagccagtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtgtttttcacctaaatttccgcgtacggtgtcaaagtccggtgtttttacgcgatcgctagcgacatcgatcacaagtttgtacaaaaaagctgaacgagaaacgtaaaatgatataaatatcaatatattaaattagattttgcataaaaaacagactacataatactgtaaaacacaacatatccagtcactatggcggccgccgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgtgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcacgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcgattcgacagatcgcgatcgcaagtgagtagtgttctggggcgggggaggacctgcatgagggccagaataactgaaatctgtgcttttctgtgtgttgcagcagcatgagcggaagcggctcctttgagggaggggtattcagcccttatctgacggggcgtctcccctcctgggcgggagtgcgtcagaatgtgatgggatccacggtggacggccggcccgtgcagcccgcgaactcttcaaccctgacctatgcaaccctgagctcttcgtcgttggacgcagctgccgccgcagctgctgcatctgccgccagcgccgtgcgcggaatggccatgggcgccggctactacggcactctggtggccaactcgagttccaccaataatcccgccagcctgaacgaggagaagctgttgctgctgatggcccagctcgaggccttgacccagcgcctgggcgagctgacccagcaggtggctcagctgcaggagcagacgcgggccgcggttgccacggtgaaatccaaataaaaaatgaatcaataaataaacggagacggttgttgattttaacacagagtctgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcatggtgttgcacaatatctttgaggaggagactgatggccacgggcagccctttggtgtaggtgtttacaaatctgttgagctgggagggatgcatgcggggggagatgaggtgcatcttggcctggatcttgagattggcgatgttaccgcccagatcccgcctggggttcatgttgtgcaggaccaccagcacggtgtatccggtgcacttggggaatttatcatgcaacttggaagggaaggcgtgaaagaatttggcgacgcctttgtgcccgcccaggttttccatgcactcatccatgatgatggcgatgggcccgtgggcggcggcctgggcaaagacgtttcgggggtcggacacatcatagttgtggtcctgggtgaggtcatcataggccattttaatgaatttggggcggagggtgccggactgggggacaaaggtaccctcgatcccgggggcgtagttcccctcacagatctgcatctcccaggctttgagctcggagggggggatcatgtccacctgcggggcgataaagaacacggtttccggggcgggggagatgagctgggccgaaagcaagttccggagcagctgggacttgccgcagccggtggggccgtagatgaccccgatgaccggctgcaggtggtagttgagggagagacagctgccgtcctcccggaggaggggggccacctcgttcatcatctcgcgcacgtgcatgttctcgcgcaccagttccgccaggaggcgctctccccccagggataggagctcctggagcgaggcgaagtttttcagcggcttgagtccgtcggccatgggcattttggagagggtttgttgcaagagttccaggcggtcccagagctcggtgatgtgctctacggcatctcgatccagcagacctcctcgtttcgcgggttgggacggctgcgggagtagggcaccagacgatgggcgtccagcgcagccagggtccggtccttccagggtcgcagcgtccgcgtcagggtggtctccgtcacggtgaaggggtgcgcgccgggctgggcgcttgcgagggtgcgcttcaggctcatccggctggtcgaaaaccgctcccgatcggcgccctgcgcgtcggccaggtagcaattgaccatgagttcgtagttgagcgcctcggccgcgtggcctttggcgcggagcttacctttggaagtctgcccgcaggcgggacagaggagggacttgagggcgtagagcttgggggcgaggaagacggactcgggggcgtaggcgtccgcgccgcagtgggcgcagacggtctcgcactccacgagccaggtgaggtcgggctggtcggggtcaaaaaccagtttcccgccgttctttttgatgcgtttcttacctttggtctccatgagctcgtgtccccgctgggtgacaaagaggctgtccgtgtccccgtagaccgactttatgggccggtcctcgagcggtgtgccgcggtcctcctcgtagaggaaccccgcccactccgagacgaaagcccgggtccaggccagcacgaaggaggccacgtgggacgggtagcggtcgttgtccaccagcgggtccaccttttccagggtatgcaaacacatgtccccctcgtccacatccaggaaggtgattggcttgtaagtgtaggccacgtgaccgggggtcccggccgggggggtataaaagggtgcgggtccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccggcggagatgcctttcaagagcccctcgtccatctggtcagaaaagacgatctttttgttgtcgagcttggtggcgaaggagccgtagagggcgttggagaggagcttggcgatggagcgcatggtctggtttttttccttgtcggcgcgctccttggcggcgatgttgagctgcacgtactcgcgcgccacgcacttccattcggggaagacggtggtcagctcgtcgggcacgattctgacctgccagccccgattatgcagggtgatgaggtccacactggtggccacctcgccgcgcaggggctcattagtccagcagaggcgtccgcccttgcgcgagcagaaggggggcagggggtccagcatgacctcgtcgggggggtcggcatcgatggtgaagatgccgggcaggaggtcggggtcaaagtagctgatggaagtggccagatcgtccagggcagcttgccattcgcgcacggccagcgcgcgctcgtagggactgaggggcgtgccccagggcatgggatgggtaagcgcggaggcgtacatgccgcagatgtcgtagacgtagaggggctcctcgaggatgccgatgtaggtggggtagcagcgccccccgcggatgctggcgcgcacgtagtcatacagctcgtgcgagggggcgaggagccccgggcccaggttggtgcgactgggcttttcggcgcggtagacgatctggcggaaaatggcatgcgagttggaggagatggtgggcctttggaagatgttgaagtgggcgtggggcagtccgaccgagtcgcggatgaagtgggcgtaggagtcttgcagcttggcgacgagctcggcggtgactaggacgtccagagcgcagtagtcgagggtctcctggatgatgtcatacttgagctgtcccttttgtttccacagctcgcggttgagaaggaactcttcgcggtccttccagtactcttcgagggggaacccgtcctgatctgcacggtaagagcctagcatgtagaactggttgacggccttgtaggcgcagcagcccttctccacggggagggcgtaggcctgggcggccttgcgcagggaggtgtgcgtgagggcgaaagtgtccctgaccatgaccttgaggaactggtgcttgaagtcgatatcgtcgcagcccccctgctcccagagctggaagtccgtgcgcttcttgtaggcggggttgggcaaagcgaaagtaacatcgttgaagaggatcttgcccgcgcggggcataaagttgcgagtgatgcggaaaggttggggcacctcggcccggttgttgatgacctgggcggcgagcacgatctcgtcgaagccgttgatgttgtggcccacgatgtagagttccacgaatcgcggacggcccttgacgtggggcagtttcttgagctcctcgtaggtgagctcgtcggggtcgctgagcccgtgctgctcgagcgcccagtcggcgagatgggggttggcgcggaggaaggaagtccagagatccacggccagggcggtttgcagacggtcccggtactgacggaactgctgcccgacggccattttttcgggggtgacgcagtagaaggtgcgggggtccccgtgccagcgatcccatttgagctggagggcgagatcgagggcgagctcgacgagccggtcgtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccaattggaggaatggctgttgatgtgatggaagtagaaatgccgacggcgcgccgaacactcgtgcttgtgtttatacaagcggccacagtgctcgcaacgctgcacgggatgcacgtgctgcacgagctgtacctgagttcctttgacgaggaatttcagtgggaagtggagtcgtggcgcctgcatctcgtgctgtactacgtcgtggtggtcggcctggccctcttctgcctcgatggtggtcatgctgacgagcccgcgcgggaggcaggtccagacctcggcgcgagcgggtcggagagcgaggacgagggcgcgcaggccggagctgtccagggtcctgagacgctgcggagtcaggtcagtgggcagcggcggcgcgcggttgacttgcaggagtttttccagggcgcgcgggaggtccagatggtacttgatctccaccgcgccattggtggcgacgtcgatggcttgcagggtcccgtgcccctggggtgtgaccaccgtcccccgtttcttcttgggcggctggggcgacgggggcggtgcctcttccatggttagaagcggcggcgaggacgcgcgccgggcggcaggggcggctcggggcccggaggcaggggcggcaggggcacgtcggcgccgcgcgcgggtaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcttgaaggtctccgcggccggcgcgctccacggtggccgcgaggtcgttggagatgcggcccatgagctgcgagaaggcgttcatgcccgcctcgttccagacgcggctgtagaccacgacgccctcgggatcgccggcgcgcatgaccacctgggcgaggttgagctccacgtggcgcgtgaagaccgcgtagttgcagaggcgctggtagaggtagttgagcgtggtggcgatgtgctcggtgacgaagaaatacatgatccagcggcggagcggcatctcgctgacgtcgcccagcgcctccaaacgttccatggcctcgtaaaagtccacggcgaagttgaaaaactgggagttgcgcgccgagacggtcaactcctcctccagaagacggatgagctcggcgatggtggcgcgcacctcgcgctcgaaggcccccgggagttcctccacttcctcttcttcctcctccactaacatctcttctacttcctcctcaggcggcagtggtggcgggggagggggcctgcgtcgccggcggcgcacgggcagacggtcgatgaagcgctcgatggtctcgccgcgccggcgtcgcatggtctcggtgacggcgcgcccgtcctcgcggggccgcagcgtgaagacgccgccgcgcatctccaggtggccgggggggtccccgttgggcagggagagggcgctgacgatgcatcttatcaattgccccgtagggactccgcgcaaggacctgagcgtctcgagatccacgggatctgaaaaccgctgaacgaaggcttcgagccagtcgcagtcgcaaggtaggctgagcacggtttcttctggcgggtcatgttggttgggagcggggcgggcgatgctgctggtgatgaagttgaaataggcggttctgagacggcggatggtggcgaggagcaccaggtctttgggcccggcttgctggatgcgcagacggtcggccatgccccaggcgtggtcctgacacctggccaggtccttgtagtagtcctgcatgagccgctccacgggcacctcctcctcgcccgcgcggccgtgcatgcgcgtgagcccgaagccgcgctggggctggacgagcgccaggtcggcgacgacgcgctcggcgaggatggcttgctggatctgggtgagggtggtctggaagtcatcaaagtcgacgaagcggtggtaggctccggtgttgatggtgtaggagcagttggccatgacggaccagttgacggtctggtggcccggacgcacgagctcgtggtacttgaggcgcgagtaggcgcgcgtgtcgaagatgtagtcgttgcaggtgcgcaccaggtactggtagccgatgaggaagtgcggcggcggctggcggtagagcggccatcgctcggtggcgggggcgccgggcgcgaggtcctcgagcatggtgcggtggtagccgtagatgtacctggacatccaggtgatgccggcggcggtggtggaggcgcgcgggaactcgcggacgcggttccagatgttgcgcagcggcaggaagtagttcatggtgggcacggtctggcccgtgaggcgcgcgcagtcgtggatgctctatacgggcaaaaacgaaagcggtcagcggctcgactccgtggcctggaggctaagcgaacgggttgggctgcgcgtgtaccccggttcgaatctcgaatcaggctggagccgcagctaacgtggtattggcactcccgtctcgacccaagcctgcaccaaccctccaggatacggaggcgggtcgttttgcaacttttttttggaggccggatgagactagtaagcgcggaaagcggccgaccgcgatggctcgctgccgtagtctggagaagaatcgccagggttgcgttgcggtgtgccccggttcgaggccggccggattccgcggctaacgagggcgtggctgccccgtcgtttccaagaccccatagccagccgacttctccagttacggagcgagcccctcttttgttttgtttgtttttgccagatgcatcccgtactgcggcagatgcgcccccaccaccctccaccgcaacaacagccccctccacagccggcgcttctgcccccgccccagcagcaacttccagccacgaccgccgcggccgccgtgagcggggctggacagagttatgatcaccagctggccttggaagagggcgaggggctggcgcgcctgggggcgtcgtcgccggagcggcacccgcgcgtgcagatgaaaagggacgctcgcgaggcctacgtgcccaagcagaacctgttcagagacaggagcggcgaggagcccgaggagatgcgcgcggcccggttccacgcggggcgggagctgcggcgcggcctggaccgaaagagggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcaccctgatcgcgcgcgaggaggtgaccctgggcctgatgcacctgtgggacctgctggaggccatcgtgcagaaccccaccagcaagccgctgacggcgcagctgttcctggtggtgcagcatagtcgggacaacgaagcgttcagggaggcgctgctgaatatcaccgagcccgagggccgctggctcctggacctggtgaacattctgcagagcatcgtggtgcaggagcgcgggctgccgctgtccgagaagctggcggccatcaacttctcggtgctgagtttgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgtgcggtgagcgccagcaggcggcgcgagctgagcgaccaggagctgatgcatagtctgcagcgggccctgaccggggccgggaccgagggggagagctactttgacatgggcgcggacctgcactggcagcccagccgccgggccttggaggcggcggcaggaccctacgtagaagaggtggacgatgaggtggacgaggagggcgagtacctggaagactgatggcgcgaccgtatttttgctagatgcaacaacaacagccacctcctgatcccgcgatgcgggcggcgctgcagagccagccgtccggcattaactcctcggacgattggacccaggccatgcaacgcatcatggcgctgacgacccgcaaccccgaagcctttagacagcagccccaggccaaccggctctcggccatcctggaggccgtggtgccctcgcgctccaaccccacgcacgagaaggtcctggccatcgtgaacgcgctggtggagaacaaggccatccgcggcgacgaggccggcctggtgtacaacgcgctgctggagcgcgtggcccgctacaacagcaccaacgtgcagaccaacctggaccgcatggtgaccgacgtgcgcgaggccgtggcccagcgcgagcggttccaccgcgagtccaacctgggatccatggtggcgctgaacgccttcctcagcacccagcccgccaacgtgccccggggccaggaggactacaccaacttcatcagcgccctgcgcctgatggtgaccgaggtgccccagagcgaggtgtaccagtccgggccggactacttcttccagaccagtcgccagggcttgcagaccgtgaacctgagccaggctttcaagaacttgcagggcctgtggggcgtgcaggccccggtcggggaccgcgcgacggtgtcgagcctgctgacgccgaactcgcgcctgctgctgctgctggtggcccccttcacggacagcggcagcatcaaccgcaactcgtacctgggctacctgattaacctgtaccgcgaggccatcggccaggcgcacgtggacgagcagacctaccaggagatcacccacgtgagccgcgccctgggccaggacgacccgggcaacctggaagccaccctgaactttttgctgaccaaccggtcgcagaagatcccgccccagtacgcgctcagcaccgaggaggagcgcatcctgcgttacgtgcagcagagcgtgggcctgttcctgatgcaggagggggccacccccagcgccgcgctcgacatgaccgcgcgcaacatggagcccagcatgtacgccagcaaccgcccgttcatcaataaactgatggactacttgcatcgggcggccgccatgaactctgactatttcaccaacgccatcctgaatccccactggctcccgccgccggggttctacacgggcgagtacgacatgcccgaccccaatgacgggttcctgtgggacgatgtggacagcagcgtgttctccccccgaccgggtgctaacgagcgccccttgtggaagaaggaaggcagcgaccgacgcccgtcctcggcgctgtccggccgcgagggtgctgccgcggcggtgcccgaggccgccagtcctttcccgagcttgcccttctcgctgaacagtatccgcagcagcgagctgggcaggatcacgcgcccgcgcttgctgggcgaagaggagtacttgaatgactcgctgttgagacccgagcgggagaagaacttccccaataacgggatagaaagcctggtggacaagatgagccgctggaagacgtatgcgcaggagcacagggacgatccccgggcgtcgcagggggccacgagccggggcagcgccgcccgtaaacgccggtggcacgacaggcagcggggacagatgtgggacgatgaggactccgccgacgacagcagcgtgttggacttgggtgggagtggtaacccgttcgctcacctgcgcccccgtatcgggcgcatgatgtaagagaaaccgaaaataaatgatactcaccaaggccatggcgaccagcgtgcgttcgtttcttctctgttgttgttgtatctagtatgatgaggcgtgcgtacccggagggtcctcctccctcgtacgagagcgtgatgcagcaggcgatggcggcggcggcgatgcagcccccgctggaggctccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaatgacttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcccgcaagacccccaatggggtgacagtgacagaggattatgatggtagtcaggatgagctgaagtatgaatgggtggaatttgagctgcccgaaggcaacttctcggtgaccatgaccatcgacctgatgaacaacgccatcatcgacaattacttggcggtggggcggcagaacggggtgctggagagcgacatcggcgtgaagttcgacactaggaacttcaggctgggctgggaccccgtgaccgagctggtcatgcccggggtgtacaccaacgaggctttccatcccgatattgtcttgctgcccggctgcggggtggacttcaccgagagccgcctcagcaacctgctgggcattcgcaagaggcagcccttccaggaaggcttccagatcatgtacgaggatctggaggggggcaacatccccgcgctcctggatgtcgacgcctatgagaaaagcaaggaggatgcagcagctgaagcaactgcagccgtagctaccgcctctaccgaggtcaggggcgataattttgcaagcgccgcagcagtggcagcggccgaggcggctgaaaccgaaagtaagatagtcattcagccggtggagaaggatagcaagaacaggagctacaacgtactaccggacaagataaacaccgcctaccgcagctggtacctagcctacaactatggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgcttacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgttactgacgccagacgccgcacctgcccctacgtctacaaggccctgggcatagtcgcgccgcgcgtcctctcgagccgcaccttctaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcggtcgcgcaccaccgtcgacgacgtgatcgaccaggtggtggccgacgcgcgcaactacacccccgccgccgcgcccgtctccaccgtggacgccgtcatcgacagcgtggtggcggacgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcttcaggcgccagcgccggcaggacccggagacgcgcggccacggcggcggcagcggccatcgccagcatgtcccgcccgcggcgagggaacgtgtactgggtgcgcgacgccgccaccggtgtgcgcgtgcccgtgcgcacccgcccccctcgcacttgaagatgttcacttcgcgatgttgatgtgtcccagcggcgaggaggatgtccaagcgcaaattcaaggaagagatgctccaggtcatcgcgcctgagatctacggccctgcggtggtgaaggaggaaagaaagccccgcaaaatcaagcgggtcaaaaaggacaaaaaggaagaagaaagtgatgtggacggattggtggagtttgtgcgcgagttcgccccccggcggcgcgtgcagtggcgcgggcggaaggtgcaaccggtgctgagacccggcaccaccgtggtcttcacgcccggcgagcgctccggcaccgcttccaagcgctcctacgacgaggtgtacggggatgatgatattctggagcaggcggccgagcgcctgggcgagtttgcttacggcaagcgcagccgttccgcaccgaaggaagaggcggtgtccatcccgctggaccacggcaaccccacgccgagcctcaagcccgtgaccttgcagcaggtgctgccgaccgcggcgccgcgccgggggttcaagcgcgagggcgaggatctgtaccccaccatgcagctgatggtgcccaagcgccagaagctggaagacgtgctggagaccatgaaggtggacccggacgtgcagcccgaggtcaaggtgcggcccatcaagcaggtggccccgggcctgggcgtgcagaccgtggacatcaagattcccacggagcccatggaaacgcagaccgagcccatgatcaagcccagcaccagcaccatggaggtgcagacggatccctggatgccatcggctcctagtcgaagaccccggcgcaagtacggcgcggccagcctgctgatgcccaactacgcgctgcatccttccatcatccccacgccgggctaccgcggcacgcgcttctaccgcggtcataccagcagccgccgccgcaagaccaccactcgccgccgccgtcgccgcaccgccgctgcaaccacccctgccgccctggtgcggagagtgtaccgccgcggccgcgcacctctgaccctgccgcgcgcgcgctaccacccgagcatcgccatttaaactttcgccagctttgcagatcaatggccctcacatgccgccttcgcgttcccattacgggctaccgaggaagaaaaccgcgccgtagaaggctggcggggaacgggatgcgtcgccaccaccaccggcggcggcgcgccatcagcaagcggttggggggaggcttcctgcccgcgctgatccccatcatcgccgcggcgatcggggcgatccccggcattgcttccgtggcggtgcaggcctctcagcgccactgagacacacttggaaacatcttgtaataaacccatggactctgacgctcctggtcctgtgatgtgttttcgtagacagatggaagacatcaatttttcgtccctggctccgcgacacggcacgcggccgttcatgggcacctggagcgacatcggcaccagccaactgaacgggggcgccttcaattggagcagtctctggagcgggcttaagaatttcgggtccacgcttaaaacctatggcagcaaggcgtggaacagcaccacagggcaggcgctgagggataagctgaaagagcagaacttccagcagaaggtggtcgatgggctcgcctcgggcatcaacggggtggtggacctggccaaccaggccgtgcagcggcagatcaacagccgcctggacccggtgccgcccgccggctccgtggagatgccgcaggtggaggaggagctgcctcccctggacaagcggggcgagaagcgaccccgccccgatgcggaggagacgctgctgacgcacacggacgagccgcccccgtacgaggaggcggtgaaactgggtctgcccaccacgcggcccatcgcgcccctggccaccggggtgctgaaacccgaaaagcccgcgaccctggacttgcctcctccccagccttcccgcccctctacagtggctaagcccctgccgccggtggccgtggcccgcgcgcgacccgggggcaccgcccgccctcatgcgaactggcagagcactctgaacagcatcgtgggtctgggagtgcagagtgtgaagcgccgccgctgctattaaacctaccgtagcgcttaacttgcttgtctgtgtgtgtatgtattatgtcgccgccgccgctgtccaccagaaggaggagtgaagaggcgcgtcgccgagttgcaagatggccaccccatcgatgctgccccagtgggcgtacatgcacatcgccggacaggacgcttcggagtacctgagtccgggtctggtgcagtttgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcggggccctagcttcaaaccctactccggcaccgcctacaacagtctggcccccaagggagcacccaacacttgtcagtggacatataaagccgatggtgaaactgccacagaaaaaacctatacatatggaaatgcacccgtgcagggcattaacatcacaaaagatggtattcaacttggaactgacaccgatgatcagccaatctacgcagataaaacctatcagcctgaacctcaagtgggtgatgctgaatggcatgacatcactggtactgatgaaaagtatggaggcagagctcttaagcctgataccaaaatgaagccttgttatggttcttttgccaagcctactaataaagaaggaggtcaggcaaatgtgaaaacaggaacaggcactactaaagaatatgacatagacatggctttctttgacaacagaagtgcggctgctgctggcctagctccagaaattgttttgtatactgaaaatgtggatttggaaactccagatacccatattgtatacaaagcaggcacagatgacagcagctcttctattaatttgggtcagcaagccatgcccaacagacctaactacattggtttcagagacaactttatcgggctcatgtactacaacagcactggcaatatgggggtgctggccggtcaggcttctcagctgaatgctgtggttgacttgcaagacagaaacaccgagctgtcctaccagctcttgcttgactctctgggtgacagaacccggtatttcagtatgtggaatcaggcggtggacagctatgatcctgatgtgcgcattattgaaaatcatggtgtggaggatgaacttcccaactattgtttccctctggatgctgttggcagaacagatacttatcagggaattaaggctaatggaactgatcaaaccacatggaccaaagatgacagtgtcaatgatgctaatgagataggcaagggtaatccattcgccatggaaatcaacatccaagccaacctgtggaggaacttcctctacgccaacgtggccctgtacctgcccgactcttacaagtacacgccggccaatgttaccctgcccaccaacaccaacacctacgattacatgaacggccgggtggtggcgccctcgctggtggactcctacatcaacatcggggcgcgctggtcgctggatcccatggacaacgtgaaccccttcaaccaccaccgcaatgcggggctgcgctaccgctccatgctcctgggcaacgggcgctacgtgcccttccacatccaggtgccccagaaatttttcgccatcaagagcctcctgctcctgcccgggtcctacacctacgagtggaacttccgcaaggacgtcaacatgatcctgcagagctccctcggcaacgacctgcgcacggacggggcctccatctccttcaccagcatcaacctctacgccaccttcttccccatggcgcacaacacggcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgtctcaagaccaaggagacgccctcgctgggctccgggttcgacccctacttcgtctactcgggctccatcccctacctcgacggcaccttctacctcaaccacaccttcaagaaggtctccatcaccttcgactcctccgtcagctggcccggcaacgaccggctcctgacgcccaacgagttcgaaatcaagcgcaccgtcgacggcgagggctacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtggtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcaccagcgtcacccagaaaaagttcctctgcgacagggtcatgtggcgcatccccttctccagcaacttcatgtccatgggcgcgctcaccgacctcggccagaacatgctctatgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacccccttctcggccggtaacgccaccacctaagctcttgcttcttgcaagccatggccgcgggctccggcgagcaggagctcagggccatcatccgcgacctgggctgcgggccctacttcctgggcaccttcgataagcgcttcccgggattcatggccccgcacaagctggcctgcgccatcgtcaacacggccggccgcgagaccgggggcgagcactggctggccttcgcctggaacccgcgctcgaacacctgctacctcttcgaccccttcgggttctcggacgagcgcctcaagcagatctaccagttcgagtacgagggcctgctgcgccgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaactcccactccgcctactttcgctcccaccgcgcgcgcatcgagaaggccaccgccttcgaccgcatgaatcaagacatgtaaaccgtgtgtgtatgttaaatgtctttaataaacagcactttcatgttacacatgcatctgagatgatttatttagaaatcgaaagggttctgccgggtctcggcatggcccgcgggcagggacacgttgcggaactggtacttggccagccacttgaactcggggatcagcagtttgggcagcggggtgtcggggaaggagtcggtccacagcttccgcgtcagttgcagggcgcccagcaggtcgggcgcggagatcttgaaatcgcagttgggacccgcgttctgcgcgcgggagttgcggtacacggggttgcagcactggaacaccatcagggccgggtgcttcacgctcgccagcaccgtcgcgtcggtgatgctctccacgtcgaggtcctcggcgttggccatcccgaagggggtcatcttgcaggtctgccttcccatggtgggcacgcacccgggcttgtggttgcaatcgcagtgcagggggatcagcatcatctgggcctggtcggcgttcatccccgggtacatggccttcatgaaagcctccaattgcctgaacgcctgctgggccttggctccctcggtgaagaagaccccgcaggacttgctagagaactggttggtggcgcacccggcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcatgtgctccttctggatcatggtggtcccgtgcaggcaccgcagcttgccctcggcctcggtgcacccgtgcagccacagcgcgcacccggtgcactcccagttcttgtgggcgatctgggaatgcgcgtgcacgaagccctgcaggaagcggcccatcatggtggtcagggtcttgttgctagtgaaggtcagcggaatgccgcggtgctcctcgttgatgtacaggtggcagatgcggcggtacacctcgccctgctcgggcatcagctggaagttggctttcaggtcggtctccacgcggtagcggtccatcagcatagtcatgatttccatacccttctcccaggccgagacgatgggcaggctcatagggttcttcaccatcatcttagcgctagcagccgcggccagggggtcgctctcgtccagggtctcaaagctccgcttgccgtccttctcggtgatccgcaccggggggtagctgaagcccacggccgccagctcctcctcggcctgtctttcgtcctcgctgtcctggctgacgtcctgcaggaccacatgcttggtcttgcggggtttcttcttgggcggcagcggcggcggagatgttggagatggcgagggggagcgcgagttctcgctcaccactactatctcttcctcttcttggtccgaggccacgcggcggtaggtatgtctcttcgggggcagaggcggaggcgacgggctctcgccgccgcgacttggcggatggctggcagagccccttccgcgttcgggggtgcgctcccggcggcgctctgactgacttcctccgcggccggccattgtgttctcctagggaggaacaacaagcatggagactcagccatcgccaacctcgccatctgcccccaccgccgacgagaagcagcagcagcagaatgaaagcttaaccgccccgccgcccagccccgccacctccgacgcggccgtcccagacatgcaagagatggaggaatccatcgagattgacctgggctatgtgacgcccgcggagcacgaggaggagctggcagtgcgcttttcacaagaagagatacaccaagaacagccagagcaggaagcagagaatgagcagagtcaggctgggctcgagcatgacggcgactacctccacctgagcgggggggaggacgcgctcatcaagcatctggcccggcaggccaccatcgtcaaggatgcgctgctcgaccgcaccgaggtgcccctcagcgtggaggagctcagccgcgcctacgagttgaacctcttctcgccgcgcgtgccccccaagcgccagcccaatggcacctgcgagcccaacccgcgcctcaacttctacccggtcttcgcggtgcccgaggccctggccacctaccacatctttttcaagaaccaaaagatccccgtctcctgccgcgccaaccgcacccgcgccgacgcccttttcaacctgggtcccggcgcccgcctacctgatatcgcctccttggaagaggttcccaagatcttcgagggtctgggcagcgacgagactcgggccgcgaacgctctgcaaggagaaggaggagagcatgagcaccacagcgccctggtcgagttggaaggcgacaacgcgcggctggcggtgctcaaacgcacggtcgagctgacccatttcgcctacccggctctgaacctgccccccaaagtcatgagcgcggtcatggaccaggtgctcatcaagcgcgcgtcgcccatctccgaggacgagggcatgcaagactccgaggagggcaagcccgtggtcagcgacgagcagctggcccggtggctgggtcctaatgctagtccccagagtttggaagagcggcgcaaactcatgatggccgtggtcctggtgaccgtggagctggagtgcctgcgccgcttcttcgccgacgcggagaccctgcgcaaggtcgaggagaacctgcactacctcttcaggcacgggttcgtgcgccaggcctgcaagatctccaacgtggagctgaccaacctggtctcctacatgggcatcttgcacgagaaccgcctggggcagaacgtgctgcacaccaccctgcgcggggaggcccggcgcgactacatccgcgactgcgtctacctctacctctgccacacctggcagacgggcatgggcgtgtggcagcagtgtctggaggagcagaacctgaaagagctctgcaagctcctgcagaagaacctcaagggtctgtggaccgggttcgacgagcgcaccaccgcctcggacctggccgacctcattttccccgagcgcctcaggctgacgctgcgcaacggcctgcccgactttatgagccaaagcatgttgcaaaactttcgctctttcatcctcgaacgctccggaatcctgcccgccacctgctccgcgctgccctcggacttcgtgccgctgaccttccgcgagtgccccccgccgctgtggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaagggcccagcgaaggcgagggttcagccgccaaggggggtctgaaactcaccccggggctgtggacctcggcctacttgcgcaagttcgtgcccgaggactaccatcccttcgagatcaggttctacgaggaccaatcccatccgcccaaggccgagctgtcggcctgcgtcatcacccagggggcgatcctggcccaattgcaagccatccagaaatcccgccaagaattcttgctgaaaaagggccgcggggtctacctcgacccccagaccggtgaggagctcaaccccggcttcccccaggatgccccgaggaaacaagaagctgaaagtggagctgccgcccgtggaggatttggaggaagactgggagaacagcagtcaggcagaggaggaggagatggaggaagactgggacagcactcaggcagaggaggacagcctgcaagacagtctggaggaagacgaggaggaggcagaggaggaggtggaagaagcagccgccgccagaccgtcgtcctcggcgggggagaaagcaagcagcacggataccatctccgctccgggtcggggtcccgctcgaccacacagtagatgggacgagaccggacgattcccgaaccccaccacccagaccggtaagaaggagcggcagggatacaagtcctggcgggggcacaaaaacgccatcgtctcctgcttgcaggcctgcgggggcaacatctccttcacccggcgctacctgctcttccaccgcggggtgaactttccccgcaacatcttgcattactaccgtcacctccacagcccctactacttccaagaagaggcagcagcagcagaaaaagaccagcagaaaaccagcagctagaaaatccacagcggcggcagcaggtggactgaggatcgcggcgaacgagccggcgcaaacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccagtcgcagaaaaaggcgggaattacgtcacctgtgcccttcgccctagccgcctccacccatcatcatgagcaaagagattcccacgccttacatgtggagctaccagccccagatgggcctggccgccggtgccgcccaggactactccacccgcatgaattggctcagcgccgggcccgcgatgatctcacgggtgaatgacatccgcgcccaccgaaaccagatactcctagaacagtcagcgctcaccgccacgccccgcaatcacctcaatccgcgtaattggcccgccgccctggtgtaccaggaaattccccagcccacgaccgtactacttccgcgagacgcccaggccgaagtccagctgactaactcaggtgtccagctggcgggcggcgccaccctgtgtcgtcaccgccccgctcagggtataaagcggctggtgatccggggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgagtttaaactcacccccttatccagtgaaataaagatcatattgatgatgattttacagaaataaaaaataatcatttgatttgaaataaagatacaatcatattgatgatttgagtttaacaaaaaaataaagaatcacttacttgaaatctgataccaggtctctgtccatgttttctgccaacaccacttcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggccgaccccgtcaccaccaagaacggggaaatcaccctcaagctgggagagggggtggacctcgattcctcgggaaaactcatctccaacacggccaccaaggccgccgcccctctcagtttttccaacaacaccatttcccttaacatggatcaccccttttacactaaagatggaaaattatccttacaagtttctccaccattaaatatactgagaacaagcattctaaacacactagctttaggttttggatcaggtttaggactccgtggctctgccttggcagtacagttagtctctccacttacatttgatactgatggaaacataaagcttaccttagacagaggtttgcatgttacaacaggagatgcaattgaaagcaacataagctgggctaaaggtttaaaatttgaagatggagccatagcaaccaacattggaaatgggttagagtttggaagcagtagtacagaaacaggtgttgatgatgcttacccaatccaagttaaacttggatctggccttagctttgacagtacaggagccataatggctggtaacaaagaagacgataaactcactttgtggacaacacctgatccatcaccaaactgtcaaatactcgcagaaaatgatgcaaaactaacactttgcttgactaaatgtggtagtcaaatactggccactgtgtcagtcttagttgtaggaagtggaaacctaaaccccattactggcaccgtaagcagtgctcaggtgtttctacgttttgatgcaaacggtgttcttttaacagaacattctacactaaaaaaatactgggggtataggcagggagatagcatagatggcactccatataccaatgctgtaggattcatgcccaatttaaaagcttatccaaagtcacaaagttctactactaaaaataatatagtagggcaagtatacatgaatggagatgtttcaaaacctatgcttctcactataaccctcaatggtactgatgacagcaacagtacatattcaatgtcattttcatacacctggactaatggaagctatgttggagcaacatttggggctaactcttataccttctcatacatcgcccaagaatgaacactgtatcccaccctgcatgccaacccttcccaccccactctgtggaacaaactctgaaacacaaaataaaataaagttcaagtgttttattgattcaacagtttcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgattaactttattccaaacggtctcggagcacttcaaaatgcaggtcccggaggtggcacctctcgcccccactgtgttggtggaaaataacagccaggtcaaaggtgacacggttctcgagatgttccacggtggcttccagcaaagcctccacgcgcacatccagaaacaagaggacagcgaaagcgggagcgttttctaattcctcaatcatcatattacactcctgcaccatccccagataattttcatttttccagccttgaatgattcgtattagttcctgaggtaaatccaagccagccatgataaaaagctcgcgcagagcgccctccaccggcattcttaagcacaccctcataattccaagagattctgctcctggttcacctgcagcagattaacaatgggaatatcaaaatctctgccgcgatccctaagctcctccctcaacaataactgtatgtaatctttcatatcatctccgaaatttttagccatagggccgccaggaataagagcagggcaagccacattacagataaagcgaagtcctccccagtgwgcattgccaaatgtaagattgaaataagcatgctggctagaccctgtgatatcttccagataactggacagaaaatcaggcaagcaatttttaagaaaatcaacaaaagaaaagtcgtccaggtgcaggtttagagcctcaggaacaacgatggaataagtgcaaggagtgcgttccagcatggttagtgtttttttggtgatctgtagaacaaaaaataaacatgcaatattaaaccatgctagcctggcgaacaggtgggtaaatcactctttccagcaccaggcaggctacggggtctccggcgcgaccctcgtagaagctgtcgccatgattgaaaagcatcaccgagagaccttcccggtggccggcatggatgattcgagaagaagcatacactccgggaacattggcatccgtgagtgaaaaaaagcgacctataaagcctcggggcactacaatgctcaatctcaattccagcaaagccaccccatgcggatggagcacaaaattggcaggtgcgtaaaaaatgtaattactcccctcctgcacaggcagcaaagcccccgctccctccagaaacacatacaaagcctcagcgtccatagcttaccgagcacggcaggcgcaagagtcagagaaaaggctgagctctaacctgactgcccgctcctgtgctcaatatatagccctaacctacactgacgtaaaggccaaagtctaaaaatacccgccaaataatcacacacgcccagcacacgcccagaaaccggtgacacactcaaaaaaatacgcgcacttcctcaaacgcccaaaactgccgtcatttccgggttcccacgctacgtcatcaaaacacgactttcaaattccgtcgaccgttaaaaacgtcacccgccccgcccctaacggtcgcccgtctctcagccaatcagcgccccgcatccccaaattcaaacacctcatttgcatattaacgcgcacaaaaagtttgaggtatattattgatgatgg

SEQ ID NO: 6 - DNA, 225 bp, Artificial Sequence
  bgh polyadenylation signal
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 7 - DNA, 1960 bp, Artificial Sequence
  Herpesvirales long CMV promoter
atcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgca

SEQ ID NO: 8 - DNA, 1872 bp, Varicella zoster; identical to SEQ ID NO: 3

SEQ ID NO: 9 - protein, 623 aa, Varicella zoster; identical to SEQ ID NO: 1